SEQUENCE LISTING (14 sequences)

SEQ ID NO: 1 - DNA, 16569 bp, homo sapiens
  b (3107)..(3107): b is a gap in order to keep this sequence aligned with previous reference sequences.
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacbttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 2 - protein, 318 aa, homo sapiens
MPMANLLLLIVPILIAMAFLMLTERKILGYMQLRKGPNVVGPYGLLQPFADAMKLFTKEPLKPATSTITLYITAPTLALTIALLLWTPLPMPNPLVNLNLGLLFILATSSLAVYSILWSGWASNSNYALIGALRAVAQTISYEVTLAIILLSTLLMSGSFNLSTLITTQEHLWLLLPSWPLAMMWFISTLAETNRTPFDLAEGESELVSGFNIEYAAGPFALFFMAEYTNIIMMNTLTTTIFLGTTYDALSPELYTTYFVTKTLLLTSLFLWIRTAYPRFRYDQLMHLLWKNFLPLTLALLMWYVSMPITISSIPPQT

SEQ ID NO: 3 - protein, 347 aa, homo sapiens
MNPLAQPVIYSTIFAGTLITALSSHWFFTWVGLEMNMLAFIPVLTKKMNPRSTEAAIKYFLTQATASMILLMAILFNNMLSGQWTMTNTTNQYSSLMIMMAMAMKLGMAPFHFWVPEVTQGTPLTSGLLLLTWQKLAPISIMYQISPSLNVSLLLTLSILSIMAGSWGGLNQTQLRKILAYSSITHMGWMMAVLPYNPNMTILNLTIYIILTTTAFLLLNLNSSTTTLLLSRTWNKLTWLTPLIPSTLLSLGGLPPLTGFLPKWAIIEEFTKNNSLIIPTIMATITLLNLYFYLRLIYSTSITLLPMSNNVKMKWQFEHTKPTPFLPTLIALTTLLLPISPFMLMIL

SEQ ID NO: 4 - protein, 513 aa, homo sapiens
MFADRWLFSTNHKDIGTLYLLFGAWAGVLGTALSLLIRAELGQPGNLLGNDHIYNVIVTAHAFVMIFFMVMPIMIGGFGNWLVPLMIGAPDMAFPRMNNMSFWLLPPSLLLLLASAMVEAGAGTGWTVYPPLAGNYSHPGASVDLTIFSLHLAGVSSILGAINFITTIINMKPPAMTQYQTPLFVWSVLITAVLLLLSLPVLAAGITMLLTDRNLNTTFFDPAGGGDPILYQHLFWFFGHPEVYILILPGFGMISHIVTYYSGKKEPFGYMGMVWAMMSIGFLGFIVWAHHMFTVGMDVDTRAYFTSATMIIAIPTGVKVFSWLATLHGSNMKWSAAVLWALGFIFLFTVGGLTGIVLANSSLDIVLHDTYYVVAHFHYVLSMGAVFAIMGGFIHWFPLFSGYTLDQTYAKIHFTIMFIGVNLTFFPQHFLGLSGMPRRYSDYPDAYTTWNILSSVGSFISLTAVMLMIFMIWEAFASKRKVLMVEEPSMNLEWLYGCPPPYHTFEEPVYMKS

SEQ ID NO: 5 - protein, 227 aa, homo sapiens
MAHAAQVGLQDATSPIMEELITFHDHALMIIFLICFLVLYALFLTLTTKLTNTNISDAQEMETVWTILPAIILVLIALPSLRILYMTDEVNDPSLTIKSIGHQWYWTYEYTDYGGLIFNSYMLPPLFLEPGDLRLLDVDNRVVLPIEAPIRMMITSQDVLHSWAVPTLGLKTDAIPGRLNQTTFTATRPGVYYGQCSEICGANHSFMPIVLELIPLKIFEMGPVFTL

SEQ ID NO: 6 - protein, 68 aa, homo sapiens
MPQLNTTVWPTMITPMLLTLFLITQLKMLNTNYHLPPSPKPMKMKNYNKPWEPKWTKICSLHSLPPQS

SEQ ID NO: 7 - protein, 226 aa, homo sapiens
MNENLFASFIAPTILGLPAAVLIILFPPLLIPTSKYLINNRLITTQQWLIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPMALAVRLTANITAGHLLMHLIGSATLAMSTINLPSTLIIFTILILLTILEIAVALIQAYVFTLLVSLYLHDNT

SEQ ID NO: 8 - protein, 261 aa, homo sapiens
MTHQSHAYHMVKPSPWPLTGALSALLMTSGLAMWFHFHSMTLLMLGLLTNTLTMYQWWRDVTRESTYQGHHTPPVQKGLRYGMILFITSEVFFFAGFFWAFYHSSLAPTPQLGGHWPPTGITPLNPLEVPLLNTSVLLASGVSITWAHHSLMENNRNQMIQALLITILLGLYFTLLQASEYFESPFTISDGIYGSTFFVATGFHGLHVIIGSTFLTICFIRQLMFHFTSKHHFGFEAAAWYWHFVDVVWLFLYVSIYWWGS

SEQ ID NO: 9 - protein, 115 aa, homo sapiens
MNFALILMINTLLALLLMIITFWLPQLNGYMEKSTPYECGFDPMSPARVPFSMKFFLVAITFLLFDLEIALLLPLPWALQTTNLPLMVMSSLLLIIILALSLAYEWLQKGLDWTE

SEQ ID NO: 10 - protein, 98 aa, homo sapiens
MPLIYMNIMLAFTISLLGMLVYRSHLMSSLLCLEGMMLSLFIMATLMTLNTHSLLANIVPIAMLVFAACEAAVGLALLVSISNTYGLDYVHNLNLLQC

SEQ ID NO: 11 - protein, 459 aa, homo sapiens
MLKLIVPTIMLLPLTWLSKKHMIWINTTTHSLIISIIPLLFFNQINNNLFSCSPTFSSDPLTTPLLMLTTWLLPLTIMASQRHLSSEPLSRKKLYLSMLISLQISLIMTFTATELIMFYIFFETTLIPTLAIITRWGNQPERLNAGTYFLFYTLVGSLPLLIALIYTHNTLGSLNILLLTLTAQELSNSWANNLMWLAYTMAFMVKMPLYGLHLWLPKAHVEAPIAGSMVLAAVLLKLGGYGMMRLTLILNPLTKHMAYPFLVLSLWGMIMTSSICLRQTDLKSLIAYSSISHMALVVTAILIQTPWSFTGAVILMIAHGLTSSLLFCLANSNYERTHSRIMILSQGLQTLLPLMAFWWLLASLANLALPPTINLLGELSVLVTTFSWSNITLLLTGLNMLVTALYSLYMFTTTQWGSLTHHINNMKPSFTRENTLMFMHLSPILLLSLNPDIITGFSS

SEQ ID NO: 12 - protein, 603 aa, homo sapiens
MTMHTTMTTLTLTSLIPPILTTLVNPNKKNSYPHYVKSIVASTFIISLFPTTMFMCLDQEVIISNWHWATTQTTQLSLSFKLDYFSMMFIPVALFVTWSIMEFSLWYMNSDPNINQFFKYLLIFLITMLILVTANNLFQLFIGWEGVGIMSFLLISWWYARADANTAAIQAILYNRIGDIGFILALAWFILHSNSWDPQQMALLNANPSLTPLLGLLLAAAGKSAQLGLHPWLPSAMEGPTPVSALLHSSTMVVAGIFLLIRFHPLAENSPLIQTLTLCLGAITTLFAAVCALTQNDIKKIVAFSTSSQLGLMMVTIGINQPHLAFLHICTHAFFKAMLFMCSGSIIHNLNNEQDIRKMGGLLKTMPLTSTSLTIGSLALAGMPFLTGFYSKDHIIETANMSYTNAWALSITLIATSLTSAYSTRMILLTLTGQPRFPTLTNINENNPTLLNPIKRLAAGSLFAGFLITNNISPASPFQTTIPLYLKLTALAVTFLGLLTALDLNYLTNKLKMKSPLCTFYFSNMLGFYPSITHRTIPYLGLLTSQNLPLLLLDLTWLEKLLPKTISQHQISTSIITSTQKGMIKLYFLSFFFPLILTLLLIT

SEQ ID NO: 13 - protein, 174 aa, homo sapiens
MMYALFLLSVGLVMGFVGFSSKPSPIYGGLVLIVSGVVGCVIILNFGGGYMGLMVFLIYLGGMMVVFGYTTAMAIEEYPEAWGSGVEVLVSVLVGLAMEVGLVLWVKEYDGVVVVVNFNSVGSWMIYEGEGSGLIREDPIGAGALYDYGRWLVVVTGWTLFVGVYIVIEIARGN

SEQ ID NO: 14 - protein, 380 aa, Homo sapiens
MTPMRKTNPLMKLINHSFIDLPTPSNISAWWNFGSLLGACLILQITTGLFLAMHYSPDASTAFSSIAHITRDVNYGWIIRYLHANGASMFFICLFLHIGRGLYYGSFLYSETWNIGIILLLATMATAFMGYVLPWGQMSFWGATVITNLLSAIPYIGTDLVQWIWGGYSVDSPTLTRFFTFHFILPFIIAALATLHLLFLHETGSNNPLGITSHSDKITFHPYYTIKDALGLLLFLLSLMTLTLFSPDLLGDPDNYTLANPLNTPPHIKPEWYFLFAYTILRSVPNKLGGVLALLLSILILAMIPILHMSKQQSMMFRPLSQSLYWLLAADLLILTWIGGQPVSYPFTIIGQVASVLYFTTILILMPTISLIENKMLKWA

(19) United States
(12) Patent Application Publication
(10) Pub. No.: US 2016/0160287 A1
(43) Pub. Date: Jun. 9, 2016

(54) METHOD FOR DETERMINING THE RISK OF DEVELOPING RADIATION-INDUCED TOXICITY AFTER EXPOSURE TO RADIATION

(71) Applicants: UNIVERSITEIT MAASTRICHT, Maastricht (NL); ADACEMISCH ZIEKENHUIS MAASTRICHT, Maastricht (NL); STICHTING MAASTRICHT RADIATION ONCOLOGY "MAASTRO-CLINIC", Maastricht (NL)

(72) Inventors: Philippe Lambin, Genappe- Bousval (BE); Georgi Ilkov Nalbantov, Maastricht (NL); Hubertus Julius Maria Smeets, Maastricht (NL); An Mieke Voets, Bilzen (BE)

(21) Appl. No.: 14/890,411

(22) PCT Filed: May 5, 2014
(86) PCT No.: PCT/EP2014/059089

§ 371 Date: Nov. 10, 2015

(30) Foreign Application Priority Data
May 13, 2013 (EP) 13167540.7

(51) Int. Cl. C12Q 1/68; A61N 5/10

(52) U.S. Cl. CPC C12Q 1/6886; A61N 5/10; C12Q 2600/156; C12Q 2600/106

(57) ABSTRACT

The invention is in the art of medical treatments, in particular the treatment of tumors with ionizing radiation. It provides means and methods for predicting whether a subject is likely to develop radiation damage upon radiotherapy. The invention provides tools that allow individualized and optimized radiation treatment of a subject in need of a radiation treatment. The invention also provides methods of determining the risk of developing severe dyspnea after radiation treatment. More in particular, the invention relates to an in vitro method for predicting the risk of developing radiation induced toxicity comprising the steps of obtaining mitochondrial DNA from a sample of a subject, determining the number of non-synonymous variations present in at least one gene encoding a mitochondrial protein, attributing a value to the number of non-synonymous variations, wherein a higher value corresponds to a higher risk of developing radiation induced lung toxicity.

### FIELD OF THE INVENTION

The invention is in the art of medical treatments, in particular the treatment of tumors with ionizing radiation. It provides means and methods for predicting whether a subject is likely to develop radiation-induced toxicity upon exposure to radiation. The invention allows the optimized treatment of a subject in need of a radiation treatment.

### BACKGROUND OF THE INVENTION

Radiation- and/or chemical-induced toxicity in non-malignant tissues may result in debilitating side effects (e.g., intestinal radiation toxicity, pneumonitis, fibrosis, dyspnea, necrosis, telangiectasia, functional impairment, secondary cancer and mucositis). Therapeutic radiation exposure, for example, utilized in bone marrow transplant and more than half of all cancer patients, plays a critical role in approximately 25% of cancer cures. In spite of advances in the ability to deliver localized radiation for the treatment of cancer, radiation toxicity in non-malignant tissue remains the most important dose-limiting factor in clinical radiation toxicity. Moreover, patients suffering from radiation induced toxicity or long-term side effects of radiation, have a poor long term prognosis even if they are cured of the malignancy for which they received radiation treatment.

The term radiation induced toxicity is commonly used to include any one of the following conditions: Anemia, Febrile neutropenia, Hemolysis, Leukocytosis, Activated partial thromboplastin time prolonged, Electrocardiogram QT corrected interval prolonged, INR increased, Acidosis, Alkalosis, Hypercalcemia, Hyperglycemia, Hyperkalemia, Hypermagnesemia, Hypernatremia, Hypertriglyceridemia, Hyperuricemia, Hypoalbuminemia, Hypocalcemia, Hypoglycemia, Hypokalemia, Hypomagnesemia, Hyponatremia, Hypophosphatemia, Acute kidney injury, Chronic kidney disease, Hemoglobinuria, Alanine aminotransferase increased, Alkaline phosphatase increased, Aspartate aminotransferase increased, Blood bilirubin increased, Cardiac troponin I increased, Cardiac troponin T increased, CD4 lymphocytes decreased, Cholesterol high, CPK increased, Creatinine increased, Fibrinogen decreased, GGT increased, Haptoglobin decreased, Hemoglobin increased, Lipase increased, Lymphocyte count decreased, Lymphocyte count increased, Neutrophil count decreased, Platelet count decreased, Serum amylase increased, White blood cell decreased and Proteinuria.

Common terminology criteria for adverse events (CTCAE) may be found online at http://evs.nci.nih.gov/ftp1/CTCAE/About.html.

Although higher radiation doses are associated with improved local control and overall survival, the current radiation practice is based on reducing the risk of developing secondary complication during or after radiation therapy to approximately 5-15%. However, in practice only a small number (15%) of patients are hypersensitive to radiation. Thus, identifying patients that are hypersensitive to radiation allows for the improvement of the radiotherapy plan, eventually selection for proton therapy or other radiation modalities and make it more optimal for the remainder of the patients.

The relevance of genetic predisposition for individual differences in radiotherapy (RT) toxicity or radiation-induced toxicity has been recognized for long (Barnett, G. C., et al. Normal tissue reactions to radiotherapy: towards tailoring treatment dose by genotype. Nat Rev Cancer 9, 134-142 (2009)). Studies using candidate gene approaches have searched for associations between single nucleotide polymorphisms (SNPs) in multiple genes and radiotoxicity, resulting in conflicting findings (Quarmby, S., et al. Association of transforming growth factor beta-1 single nucleotide polymorphisms with radiation-induced damage to normal tissues in breast cancer patients. Int J Radiat Biol 79, 137-143 (2003), Andreassen, C. N., Alsner, J., Overgaard, M. & Overgaard, J. Prediction of normal tissue radiosensitivity from polymorphisms in candidate genes. Radiother Oncol 69, 127-135 (2003), Azria, D., et al. Single nucleotide polymorphisms, apoptosis, and the development of severe late adverse effects after radiotherapy. Clin Cancer Res 14, 6284-6288 (2008), Barnett, G. C., et al. Independent validation of genes and polymorphisms reported to be associated with radiation toxicity: a prospective analysis study. Lancet Oncol 13, 65-77 (2012), Voets, A. M., et al. No association between TGF-beta1 polymorphisms and radiation-induced lung toxicity in a European cohort of lung cancer patients. Radiotherapy and oncology: Journal of the European Society for Therapeutic Radiology and Oncology 105, 296-298 (2012)).

There is still a need in the art for more reliable markers that predict the risk of developing radiation-induced toxicity or radiation damage, in particular in the treatment of radiation-curable diseases such as thoracic cancers, such as for instance lung cancer and breast cancer. Such markers would be useful in guiding the physician towards a prediction of the radiation dose that may be applied to a certain subject without inducing damage to the body.

### SUMMARY OF THE INVENTION

A set of 321 lung cancer patients were studied herein, as well as a group of 21 patients with breast cancer. Both groups received radiation therapy. Radiation induced toxicity was determined in all patients. In the lung cancer group we scored the occurrence of dyspnea, or shortness of breath, according to the CTCAEv3.0 criteria whereas in the breast cancer group, we scored fibrosis according to the LENT-SOMA criteria (Int J Radiat Oncol Biol Phys. 1995 Mar. 30; 31(5):1049-91).

We found that an increased number of non-synonymous variations within mitochondrial genes encoding proteins which occurred at positions that were less than 90% conserved, correlated with an increased risk for radiation-induced toxicity.

We conclude that mitochondrial DNA variation can successfully predict whether a subject will develop radiation-induced toxicity after ionizing radiotherapy. In particular the non-synonymous variations (nucleotide variations resulting in a change of an amino acid in a protein encoded by a mitochondrial gene) appeared to have a predicting power for the development of radiation-induced toxicity after radiation therapy, in particular when these mutations occurred at a position that was less than 90% conserved.

Hence, the invention relates to a method for predicting the risk of developing radiation induced toxicity comprising the steps of: obtaining mitochondrial DNA from a sample of a subject and determining the number of non-synonymous variations present in at least one gene encoding a mitochondrial protein, wherein said non-synonymous variations occur at positions that are less than 90% conserved, wherein a higher number of non-synonymous variations corresponds to a higher risk of developing radiation induced toxicity.

When the data obtained in our population of lung cancer patients were expressed in a ROC curve, this resulted in an AUC of 0.770 for one set and 0.725 for the other set. When the data obtained in our population of breast cancer patients were expressed in a ROC curve, this resulted in an AUC of 0.714 (FIG. 7).

This shows that the method as disclosed herein provides a valuable and reliable tool for the prediction of radiation induced toxicity. The method according to the invention has a significantly better predictive power than the current gold standard for radiation therapy patients (mean lung dose, AUC 0.457 in our dataset). Incorporation of mtDNA information in personalized radiation therapy planning might eventually allow for escalating doses in patients predicted to be at low risk for radiation induced toxicity, improving their chance of disease free survival.

### DETAILED DESCRIPTION OF THE INVENTION

The invention will herein be first described for a group of 321 lung cancer patients that were treated with radiation therapy in two different hospitals.

Lung cancer patients undergoing ionizing radiation therapy have an increased chance of developing radiation induced toxicity in their lungs, so-called radiation induced lung toxicity (RILT). The term RILT is herein defined as the development of dyspnea after irradiation. In the examples as provided herein the term RILT is used to indicate dyspnea grade≧2 (CTCAEv3.0) within 6 months after radiation treatment.

As used herein, the phrases “radiation toxicity” or “radiation-induced toxicity” refer to radiation-induced injury to a cell or tissue arising from exposure to radiation. Radiation induces mainly DNA damage and oxidative damage, direct or indirect. The inflammation, scarring (collagen deposition) etc are mainly due to secondary repair mechanisms (removing damaged cells, closing gaps etc. Phenotypically, radiation-induced injury includes one or more of the following: structural radiation injury to a cell or tissue, increased neutrophil infiltration, increased collagen type III deposition, and increased smooth muscle cell proliferation relative to that seen in a cell or tissue not exposed to radiation. More in particular, the term radiation induced toxicity is commonly used to include any one of the following conditions: Anemia, Febrile neutropenia, Hemolysis, Leukocytosis, Activated partial thromboplastin time prolonged, Electrocardiogram QT corrected interval prolonged, INR increased, Acidosis, Alkalosis, Hypercalcemia, Hyperglycemia, Hyperkalemia, Hypermagnesemia, Hypernatremia, Hypertriglyceridemia, Hyperuricemia, Hypoalbuminemia, Hypocalcemia, Hypoglycemia, Hypokalemia, Hypomagnesemia, Hyponatremia, Hypophosphatemia, Acute kidney injury, Chronic kidney disease, Hemoglobinuria, Alanine aminotransferase increased, Alkaline phosphatase increased, Aspartate aminotransferase increased, Blood bilirubin increased, Cardiac troponin I increased, Cardiac troponin T increased, CD4 lymphocytes decreased, Cholesterol high, CPK increased, Creatinine increased, Fibrinogen decreased, GGT increased, Haptoglobin decreased, Hemoglobin increased, Lipase increased, Lymphocyte count decreased, Lymphocyte count increased, Neutrophil count decreased, Platelet count decreased, Serum amylase increased, White blood cell decreased and Proteinuria.

Common terminology criteria for adverse events (CTCAE) may be found online at http://evs.nci.nih.gov/ftp1/CTCAE/About.html.

Although higher radiation doses to a tumor, such as a lung tumor are associated with improved local control and overall survival, the current radiation practice is based on reducing the risk of developing secondary complication in the lungs (such as dyspnea) during or after radiation therapy to approximately 5-15%. However, in practice only a small number of patients are hypersensitive to radiation. Thus, identifying patients that are hypersensitive to radiation would allow for an improved treatment of the non-hypersensitive subjects wherein an increased dose may be given to patients that are not at risk of developing radiation induced toxicity such as RILT thereby increasing their chances on a complete cure.

We found that mitochondrial DNA variation can successfully predict whether lung cancer patients will develop radiation induced toxicity such as lung toxicity (RILT) after ionizing radiotherapy. We use the definition of RILT according to the CTCAEv3.0 scoring system. In essence, RILT is defined as an increase in dyspnea (shortness of breath) score. Approximately 15% of all lung cancer patients develop RILT after radiation therapy.

Even more importantly, the risk of radiation induced toxicity such as RILT may be used to determine the total dose to be applied in the therapy of a given patient. Since the success of the therapy depends on the dose provided, the treating physician is always looking for the highest possible dose to be administered.

The more accurate a prediction of radiation induced toxicity is, the higher the dose can be, thereby improving the success rate of the treatment.

We present herein a predictive model based on mitochondrial DNA (mtDNA) data that achieves an accuracy of 77%, exceeding the accuracy of existing models which achieve an accuracy of about 50-60%, which is hardly better than random.

Mitochondrial DNA is the DNA located in organelles called mitochondria, structures within eukaryotic cells that convert the chemical energy from food into a form that cells can use, adenosine triphosphate (ATP). Most of the rest of the DNA present in eukaryotic cells can be found in the cell nucleus.

In humans, mitochondrial DNA can be regarded as the smallest chromosome coding for only 37 genes and containing only about 16,600 base pairs. Human mitochondrial DNA was the first significant part of the human genome to be sequenced. In most species, including humans, mtDNA is inherited solely from the mother.

The DNA sequence of mtDNA has been determined from a large number of organisms and individuals (including some organisms that are extinct), and the comparison of those DNA sequences represents a mainstay of phylogenetics, in that it allows biologists to elucidate the evolutionary relationships among species. It also permits an examination of the relatedness of populations, and so has become important in anthropology and field biology.

For human mitochondrial DNA 100-10,000 separate copies of mtDNA are usually present per cell. In mammals, each double-stranded circular mtDNA molecule consists of 15,000-17,000 base pairs. The two strands of mtDNA are differentiated by their nucleotide content with the guanine rich strand referred to as the heavy strand or H-strand, and the cytosine rich strand referred to as the light strand or L-strand. The heavy strand encodes 28 genes, and the light strand encodes 9 genes for a total of 37 genes. Of the 37 genes, 13 are for proteins (polypeptides), 22 are for transfer RNA (tRNA) and two are for the small and large subunits of ribosomal RNA (rRNA).

The revised Cambridge Reference Sequence (rCRS) of the human mitochondrial DNA is used herein as published under NCBI number NC_012920 (Genbank's RefSeq database). It is the Cambridge Reference Sequence as provided by Anderson et al., Nature (1981) 290; 457-465 as revised by Andrews et al., Nature Genetics (1999) 2; 147. The rCRS is referred herein as the reference sequence and the nucleotide sequence of its L-strand is provided herein as SEQ ID NO: 1.

Variation in the mtDNA sequence is common and every individual carries between 10 and 60 variants. We now found that certain types of mtDNA variants are associated with radiation induced toxicity such as lung toxicity (RILT). We also found that certain types of mtDNA variations were indicative for the risk of developing radiation induced toxicity.

The terms “predicting the risk” “indicative of the risk” or “risk factor” or equivalent, as used herein, refer to assessing the probability according to which the subject as referred to herein will experience radiation induced toxicity, such as RILT. More preferably, the risk/probability of developing radiation induced toxicity within a certain time window is predicted. In a preferred embodiment of the present invention, the predictive window, preferably, is an interval of at least 3 months, such as at least 6 months, at least 9 months, at least 1 year, at least 2 years, at least 3 years, at least 4 years, at least 5 years, at least 10 years, at least 15 years or any intermitting time range. In a particular preferred embodiment of the present invention, the predictive window, preferably, is an interval of at least 6 months. Preferably, said predictive window is calculated from the time point at which radiotherapy started.

Mitochondrial DNA, extracted from blood lymphocytes of patients before they underwent radiotherapy, was sequenced using the commercially available Affymetrix Mitochondrial DNA resequencing chip 2.0 (Mitochip). The sequence obtained was compared with the reference sequence (SEQ ID NO:1) and variants were scored and validated.

It was found that in particular the non-synonymous variants in any of the protein encoding genes in the mitochondrial genome correlated with the development of radiation induced toxicity such as RILT upon radiation.

The mtDNA may be obtained from all kinds of samples by using standard techniques, commercially available in the art. Preferred is the use of samples containing nucleated cells, preferably blood cells, more preferably, white blood cells. Also preferred is the use of a sample obtained before the start of the irradiation.

The sequence of the mtDNA may be determined with routine methods which are commercially available in the art. The obtained mtDNA sequences may then be compared with a reference sequence, such as SEQ ID NO:1, and variations may be determined.

Due to the degeneracy of the genetic code, not every variation in the nucleotide sequence will automatically lead to an alteration or variant in the protein sequence. The variations in the nucleotide sequence that lead to alterations (variants) of the encoded proteins are termed herein “non-synonymous” variations. Silent variations that do not result in the alteration of the primary protein sequence are termed “synonymous” variants or variations.

The 13 mtDNA genes encoding proteins are termed ND1, ND2, CO1, CO2, ATP8, ATP6, CO3, ND3, ND4L, ND4, ND5, ND6, and CytB. Their amino acid sequence (SEQ ID NO: 2-14) is provided in tables 1-13. The position in the mtDNA genome of these 13 genes is shown in table 14.

A person skilled in the art will be able to determine whether a certain variation in the mtDNA found in a particular subject is within the coding sequence of a mitochondrial gene encoding a protein. For that purpose, he may align the sequence obtained from the subject with the reference sequence and determine whether the variant is within the range between the first and the last coding sequence of any of the genes listed in table 14.

A variant nucleotide is defined herein as a nucleotide that is different from its counterpart nucleotide at the same position in a reference sequence, such as SEQ ID NO: 1. So, a nucleotide G at position 3600 in the sequence of the mtDNA of a subject is a variant nucleotide since position 3600 in the reference sequence (SEQ ID NO:1) is a C.

If the variant nucleotide is within the coding sequence of any of the genes encoding a protein (protein genes) then the triplet encoding the corresponding amino acid may be determined. The skilled person will know how to determine the corresponding amino acid position. In detail: he may first determine the position of the variant (N) relative to the start codon by subtracting the first coding nucleotide position (FCNP) position from the position number of N. For example, a variation at position N=3340 is within the ND1 gene (Table 14) at relative position 3340−3308=32.

Next, he may determine the triplet codon number for that position by dividing the absolute value of the relative position number by 3, adding 1 to the outcome and rounding down the value towards zero. For example, relative positions 30 to 32 are within triplet number 11 of the ND1 gene. Triplet 11 encodes amino acid number 11 of the ND1 gene.

A formula for that calculation in Microsoft Excel would read: ROUNDDOWN(((ABS(N−FNP))/3+1);0). More suitable computer programs for converting nucleotide variations into amino acid variations are available in the art and are preferred.

Once the triplet codon number has been determined, it may be determined whether the nucleotide variation will result in an amino acid variation by comparing the encoded amino acid with the reference sequence (SEQ ID NO: 1) and the sequence in tables 1-13. Care is to be taken in this process to use the genetic code for mitochondrial DNA which is slightly different from the code for nuclear DNA. The codon usage table for mitochondrial DNA is known and provided herein in FIG. 1. If the nucleotide variation does not alter the amino acid encoded, the variation is referred to as a synonymous variation, otherwise it is considered a non-synonymous variation.

We found that there is a positive correlation between the number of non-synonymous variations and the risk of developing radiation induced toxicity such as RILT after radiation. The raw data obtained with the largest population of 321 lung cancer patients are shown in Table 16 below. The results obtained with the smaller population of 66 lung cancer patients were comparable if not identical. It is shown in table 16 that the number of non-synonymous variations in these patients ranges from 0 to 14. When the patients were grouped according to their number of non-synonymous variations, and groups with less than 3 members were left out, it can be seen that the risk on RILT increases with increasing number of non-synonymous variations (table 17 and FIG. 2).

TABLE 17
The risk on RILT increases with increasing
number of non-synonymous variations.

| # non- | |
|---|---|
| synonymous | % patients |
| variations | developing RILT |
| 2 | 16% |
| 3 | 17% |
| 4 | 22% |
| 5 | 17% |
| 6 | 37% |
| 7 | 32% |
| 8 | 37% |
| 9 | 20% |
| 10 | 36% |
| 11 | 20% |

In a ROC curve produced with our data as disclosed herein, the use of this “marker” in a method according to the invention resulted in an accuracy of 60% which is an improvement over prior art methods.

Hence, the invention relates to a method for predicting the risk of developing radiation induced toxicity (such as RILT) comprising the steps of obtaining mitochondrial DNA from a sample of a subject and determining the number of non-synonymous variations present in at least one gene encoding a mitochondrial protein, wherein a higher number of non-synonymous variations corresponds to a higher risk of developing radiation induced toxicity, such as lung toxicity.

When this method is combined with methods employing other or further risk factors, this may advantageously be done in a so-called nomogram. Therein, a value is attributed to a certain risk factor (such as the number of non-synonymous variations in the mitochondrial DNA) that is then converted in a risk factor, wherein a higher value indicates a higher risk.

The invention therefore also relates to a method for predicting the risk of developing radiation induced toxicity comprising the steps of obtaining mitochondrial DNA from a sample of a subject, determining the number of non-synonymous variations present in at least one gene encoding a mitochondrial protein and attributing a value to the number of non-synonymous variations, wherein a higher value corresponds to a higher risk of developing radiation induced toxicity, such as lung toxicity.

We further found that the method could be improved by scoring only those non-synonymous variations that occurred in the mtDNA genome at positions that were less conserved in evolution. For the determination of the evolutionary conservation of the individual positions in the mtDNA genome, we used a database provided online at http://mtsnp.tmig.or.jp/mtsnp/search_mtSAP_evaluation_e.html. Therein, the entire mtDNA genome of 61 mammalian species can be found. We used those data to determine the evolutionary conservation of each amino acid encoded by the reference sequence of the mtDNA (SEQ ID NO:1). The results are shown in tables 1-13 in the column Percentage Conservation (% cons).

Particularly good results were thus obtained when the non-synonymous variants that were well conserved in the reference sequence were excluded from the analysis. It was found that amino acid variants on positions that were evolutionary less conserved in the reference sequence were better pedictors for radiation induced toxicity, such as RILT than the variants on well conserved positions.

For example, amino acid number 11 of the ATP6 gene (Table 1) is 13.1% conserved and would therefore have a better predictive value than amino acid number 1 which is 98.4% conserved.

The invention therefore relates to a method as described above wherein said non-synonymous variations are selected from the group of variants that are less than 100% conserved.

Less than 100% in the present context means 99% or less, such as 98%, 97%, 96%, 95%, 94%, 93%, 92%, 91%, 90%, 89%, 88%, 87%, 86%, 85%, 84%, 83%, 82%, 81%, 80% or less conserved, such as 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50% or less, including any number between 49 and 10.

It was found that there is an optimum number of conservation to be used in the present invention. If the percentage of conservation decreases, the number of non-synonymous amino acid variations that can be used in the analysis also decreases, making the prediction increasingly more unreliable. On the other hand, the predictive value of the individual variant increases with decreasing percentage of conservation. That mechanism results in an optimal percentage of conservation that yields the optimal predictive value. In our test population that optimal value was found to be between less than 85-95%, such as less than or equal to 90% (<=90%).

Since our test population is sufficiently large, this percentage conservation is expected to be an adequate cut-off value for performing the method in any other population.

In a preferred embodiment, the invention therefore relates to a method as described above wherein said non-synonymous variations occur at positions that are at most 90% conserved (<=90% conserved).

This is illustrated by the data presented in Table 18 and FIG. 3.

TABLE 18
The risk on RILT increases with increasing number of non-synonymous
variations at positions <=90% conserved.

| # non- | |
|---|---|
| synonymous variations at | % patients |
| positions <=90% conserved | developing RILT |
| 2 | 15% |
| 3 | 16% |
| 4 | 33% |
| 5 | 32% |
| 6 | 23% |
| 7 | 43% |
| 8 | 36% |
| 9 | 22% |
| 10 | 38% |
| 11 | 33% |

The method was found to be particularly useful in a population of patients suffering from lung cancer. Hence, in a preferred embodiment, the invention relates to a method as described above wherein the subject is diagnosed with lung cancer.

The predictive value of the method as disclosed above, could even be further improved when the value attributed to the number of non-synonymous variations was added to a value obtained from clinical parameters. The most useful clinical parameter in this respect was the baseline dyspnea score.

The term baseline dyspnea score in this context means the shortness-of-breath before the start of the radiation therapy, scored qualitatively on a 0-5 scale according to the Common Toxicity Criteria version 3.0 (CTCAEv3.0). Of course, different CTC versions may also be used, as well as different approaches to score dyspnea, such as scoring according to other schemes (example: RTOG) or using quantitative measures to score dyspnea (such as measuring the movement of patients though time, using for instance a pedometer device).

The invention therefore relates to a method as described above, additionally comprising a step of attributing a value to the baseline dyspnea score of the subject and adding that value to the value obtained from the number of non-synonymous variations to obtain an aggregated value, wherein a higher aggregated value corresponds to a higher risk of developing radiation induced toxicity, such as lung toxicity.

The values attributed to the number of non-synonymous variations and the value attributed to the baseline dyspnea score of the subject may advantageously be represented in a nomogram. Such a nomogram is shown in FIG. 5.

A nomogram is a tool to estimate outcome probability by assigning points (upperscale) to each predictor value. The sum of these scores corresponds to an event probability (bottom two scales).

We also found that a method as described above could even be further improved when variations in the genes encoding tRNAs were also taken into account, in particular those variations that occur in the loops of the tRNAs.

Table 15 shows the nucleotide positions of loops of tRNAs. There are three loops; the D-loop, the T-loop and the anti-CD loop. The D-loop in tRNA contains the modified nucleotide dihydrouridine. It is composed of 7 to 11 bases and is closed by a Watson Crick base pair. The TψpC-loop (generally called the T-loop) contains thymine, a base usually found in DNA and pseudouracil (ψ). The D-loop and T-loop form a tertiary interaction in tRNA. The anticodon loop consists of 7 nucleotides that encompass the three nucleotides that correspond to the three bases of the codon of the mRNA.

The correlation between number of variants in the loops of tRNA genes is shown in table 19 and FIG. 4.

TABLE 19
The risk on RILT increases with increasing number of
variations at positions encoding the loops of tRNAs.

| # variations at positions | % patients |
|---|---|
| encoding tRNA loops | developing RILT |
| 0 | 20% |
| 1 | 33% |
| 2 | 36% |

In a preferred embodiment, the invention therefore relates to a method as described above wherein the number of variant nucleotides in any of the three loops is counted and a value attributed to the number of variants. The higher the number of variants, the higher the attributed value. The value thus obtained may then be added to the aggregated value obtained from the non-synonymous variants and the baseline dyspnea score.

In a particularly preferred embodiment, the invention therefore relates to a method as described above, additionally comprising a step of determining the number of variations in tRNA loop positions, attributing a value to the number of variations in tRNA loop positions and adding that value to the value obtained from the number of non-synonymous variations or the aggregated value.

In an even more preferred embodiment, the treatment status of the subject is also taken into account. Patients treated with chemotherapy had a higher risk of radiation induced toxicity such as RILT after the radiation treatment. This risk increased with the dose of chemotherapy. Treatment may therefore also successfully be integrated in a method according to the invention. For that purpose, a value is attributed to the treatment status, proportional to the dose of chemotherapy and that value is added to the value obtained from the number of non-synonymous variations or the aggregated value.

As detailed above, the mitochondrial genome consists of 13 genes encoding 13 different proteins. The method may be performed by considering only the non-synonymous variations in one of these genes, at each position or at some selected positions, such as less conserved positions. However, the accuracy and reliability of the method improves when more than one gene is included in the analysis, such as 2 genes or more than 2 genes, such as 3 or 4, 5, 6, 7, 8, 9, 10, 11, 12 or all protein encoding genes.

FIG. 6 shows a ROC curve obtained when a preferred method according to the invention was applied to the population of the present study. At a certain point in the ROC curve, 64 patients are predicted to not have RILT after radiation treatment whereas 5 patients do.

Therefore, when using this point for predicting whether a patient could receive higher radiation doses, these patients have a relative risk of 8% for developing RILT. This is significant improvement (p=0.012, Chi-square equality-of-proportions test) from the 24% of patients developing RILT in the whole training dataset. Furthermore, at another point in the ROC curve, 23 patients were predicted to develop RILT and could be selected for treatment with expensive radio-protectors, such as amifostine, or proton therapy. Although at that point 5 (22%) patients were wrongly predicted to be radiosensitive, this proportion is significantly less (p<0.001, Chi-square test) than the 76% of patients not developing RILT in the whole training dataset. The expenses of (over)treating such small proportion of patients are substantially smaller than when all patients would be included (increased relative benefit).

The model as described above does not only work for lung cancer patients or radiation induced lung toxicity. When we applied the same model to patients diagnosed with breast cancer, we found that the model predicted the occurrence of radiation induced toxicity with great precision and accuracy.

In a population of 21 patients with breast cancer, we obtained mitochondrial DNA from fibroblasts of each patient before the start of the radiotherapy and determined the number of non-synonymous variations present in at least one gene encoding a mitochondrial protein, wherein said non-synonymous variations occur at positions that are less than 90% conserved. We correlated these findings with the radiation induced toxicity data from these patients, in this case measured as fibroblast induced fibrosis, scored quantitatively according to the LENT/SOMA criteria (Herskind et al., Radiother. Oncol. 1998; 47: 263-269, Pavy J J, Int J Radiat Oncol Bid Phys. 1995 Mar. 30; 31(5):1049-91). Endpoint was a grade of fibrosis of at least 2 (>=2).

We found an excellent correlation between the prediction of the model and the actual fibrosis observed in the patient group (Table 20).

TABLE 20
Correlation between the prediction of the method
according to the invention and the radiation induced
toxicity observed in the patient group.

| | Method of Invention | | |
|---|---|---|---|
| Fibrosis | High risk | Low risk | Total |
| >=2 | 8 | 2 | 10 |
| <2 | 2 | 9 | 11 |
| Total | 10 | 11 | 21 |

From the data provided in table 20, it may be concluded that the accuracy of the method according to the invention is 17/21 or 81%. The positive predictive value is 8/10 or 80%, whereas the negative predictive value is 9/11 or 82%. Specificity of the method in this group was 9/11 or 82% and sensitivity was 8/10 or 80%.

The possibility to predict if patients will be hypersensitive to radiotherapy or not will allow more optimal delivery and ultimately the usage of higher radiation doses (and thus better tumor control and outcome) in patients that are not hypersensitive. This invention will predominantly be useful in radiotherapy centers and clinics that treat cancer patients, such as lung cancer patients and breast cancer patients. The invention may also be useful in the area of radiation protection, such as screening of subjects for working or living in an area with increased radiation, such as for the screening of pilots, astronauts or workers in a radiation contaminated area or nuclear plant.

### LEGEND TO THE FIGURES

FIG. 1. Codon table for mitochondrial DNA.

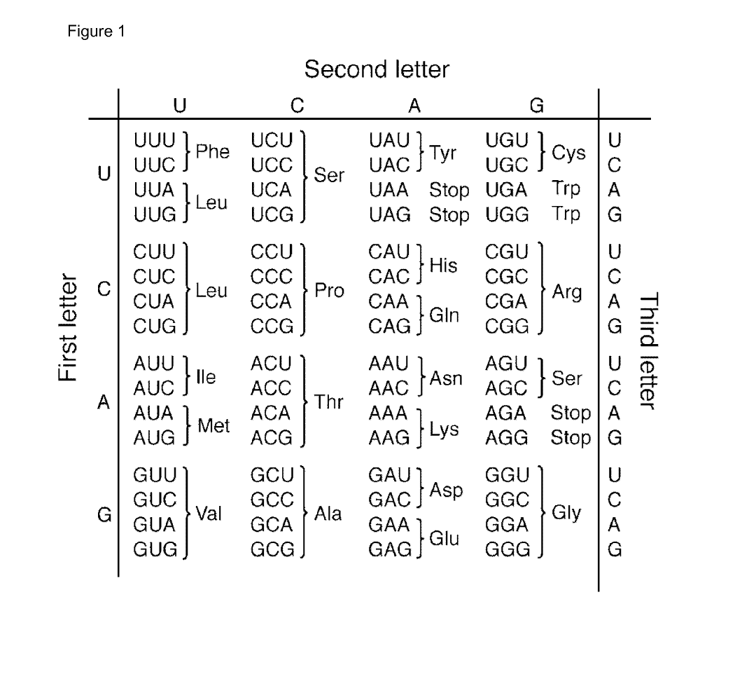

FIG. 2. Scatter plot with trendline. The figure shows the data presented in table 17. It can be seen that the risk on RILT increases with increasing number of non-synonymous variations.

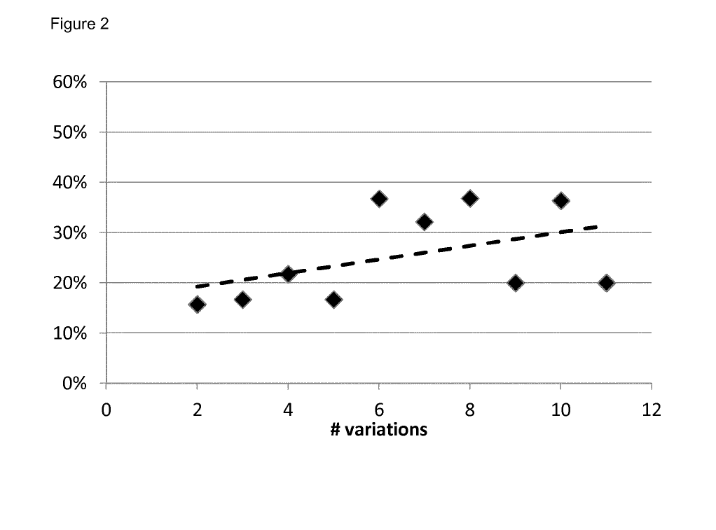

FIG. 3. Scatter plot with trendline. The figure shows the data presented in table 18. It can be seen that the risk on RILT increases with increasing number of non-synonymous variations at positions that are 90% or less conserved.

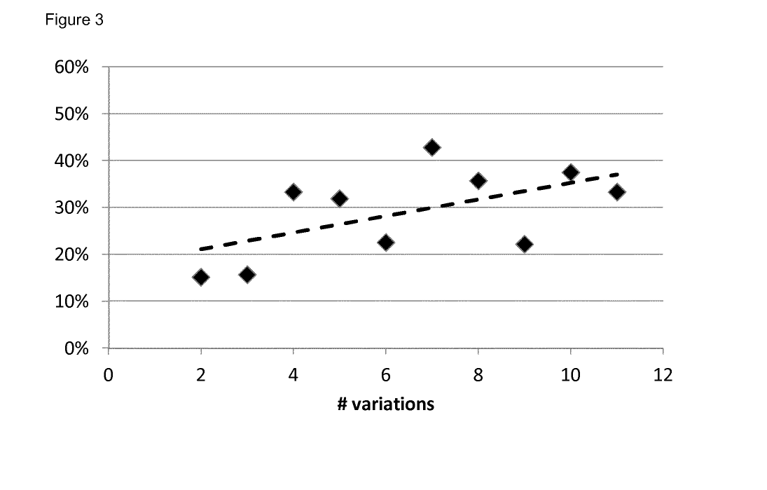

FIG. 4. Scatter plot with trendline. The figure shows the data presented in table 19. It can be seen that the risk on RILT increases with increasing number variations in the genes encoding tRNAs at position encoding the tRNA loops.

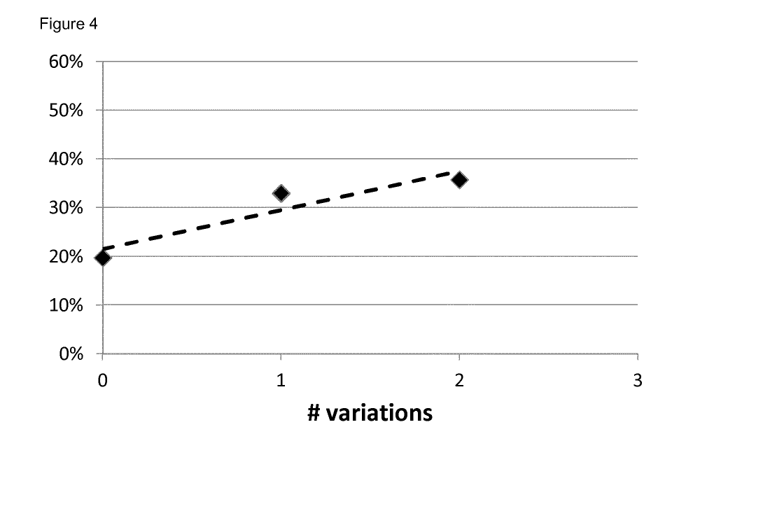

FIG. 5. The nomogram is a model visualizing a diagram for determining the probability of RILT. The figure shows a model for determining the probability of dyspnea>=2. The uppermost horizontal line indicates the number of points to be attributed to a certain parameter. For example: if 0 variations are found in the tRNA loop of an individual, then 0 points are scored. When 15 non-synonymous variations are found at positions less than 90% conserved, this accounts for an additional 42 points. A baseline dyspnea score of 1 accounts for 27 points. The total points for this individual would then be 42 plus 14 plus 0 is 56 total points. This individual would then score a probability of about 60% of RILT, calculated based on the total points horizontal line and the respective value of max. dyspnea>=2 line.

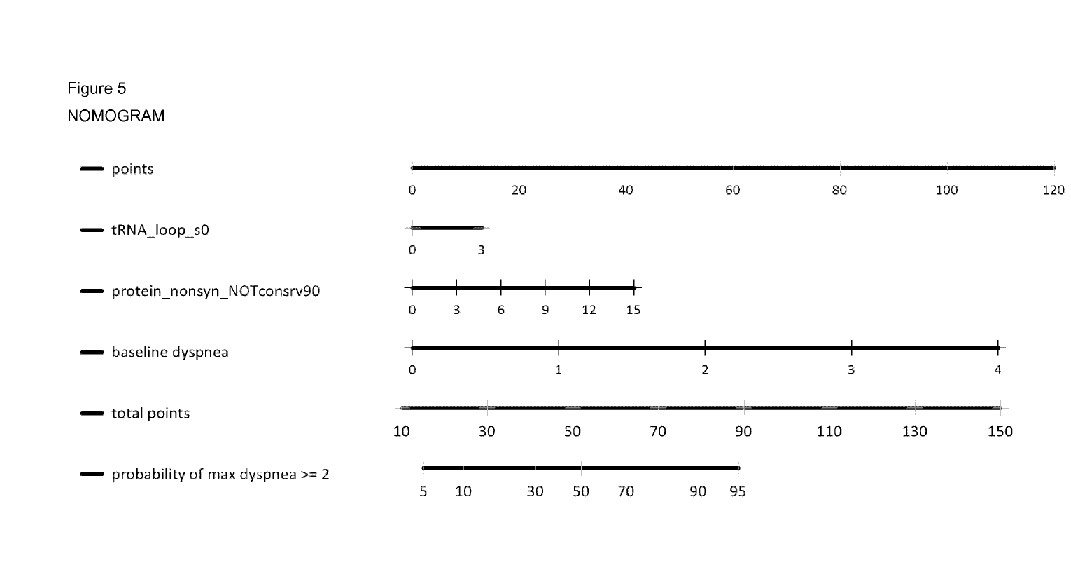

FIG. 6: ROC CURVE obtained from the population of lung cancer patients tested herein.

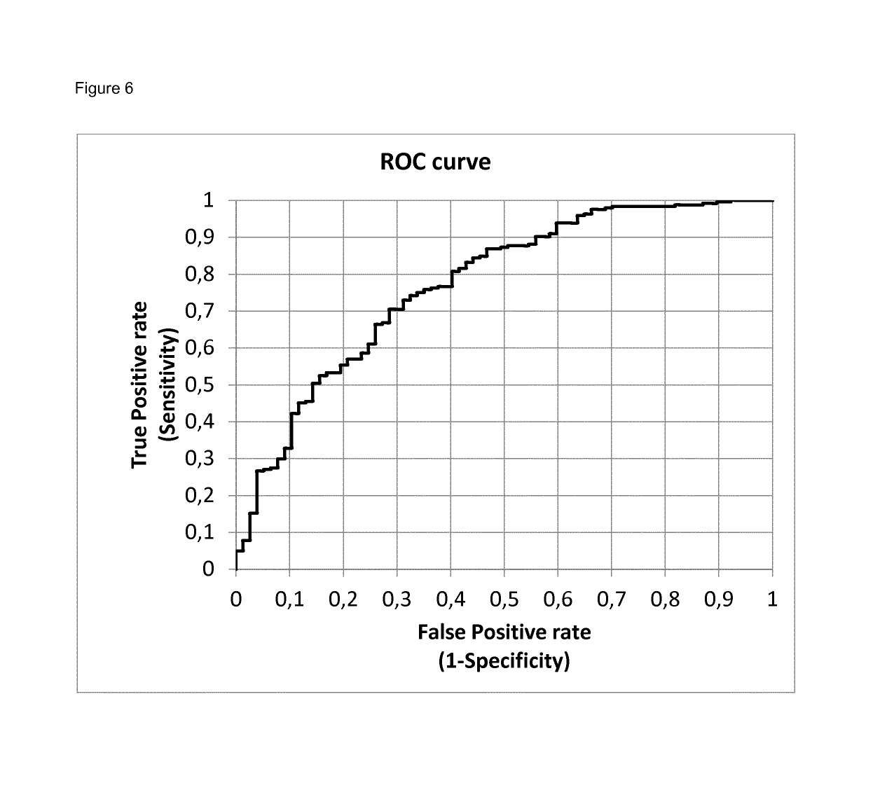

FIG. 7: ROC CURVE obtained from the population of breast cancer patients tested herein.

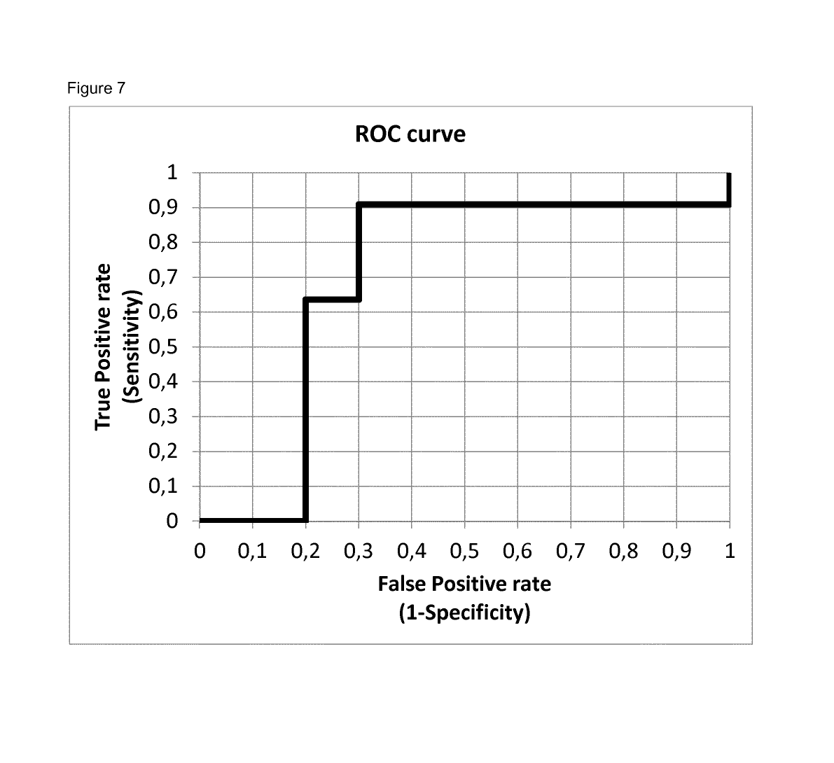

TABLE 1
ATP6 gene.

| AA# | AA | % cons. |
|---|---|---|
| 1 | M | 98.4 |
| 2 | N | 96.7 |
| 3 | E | 95.1 |
| 4 | N | 91.8 |
| 5 | L | 100.0 |
| 6 | F | 98.4 |
| 7 | A | 65.6 |
| 8 | S | 78.7 |
| 9 | F | 100.0 |
| 10 | I | 57.4 |
| 11 | A | 13.1 |
| 12 | P | 100.0 |
| 13 | T | 81.7 |
| 14 | I | 30.0 |
| 15 | L | 33.3 |
| 16 | G | 98.3 |
| 17 | L | 67.2 |
| 18 | P | 86.9 |
| 19 | A | 14.8 |
| 20 | A | 13.1 |
| 21 | V | 19.7 |
| 22 | L | 70.5 |
| 23 | I | 88.5 |
| 24 | I | 78.7 |
| 25 | L | 29.5 |
| 26 | F | 91.8 |
| 27 | P | 98.4 |
| 28 | P | 9.8 |
| 29 | L | 36.1 |
| 30 | L | 82.0 |
| 31 | I | 13.1 |
| 32 | P | 80.3 |
| 33 | T | 41.0 |
| 34 | S | 32.8 |
| 35 | K | 29.5 |
| 36 | Y | 4.9 |
| 37 | L | 83.6 |
| 38 | I | 73.8 |
| 39 | N | 65.6 |
| 40 | N | 100.0 |
| 41 | R | 98.4 |
| 42 | L | 68.9 |
| 43 | I | 45.9 |
| 44 | T | 26.2 |
| 45 | T | 13.1 |
| 46 | Q | 100.0 |
| 47 | Q | 80.3 |
| 48 | W | 93.4 |
| 49 | L | 86.9 |
| 50 | I | 55.7 |
| 51 | K | 21.3 |
| 52 | L | 86.9 |
| 53 | T | 47.5 |
| 54 | S | 47.5 |
| 55 | K | 100.0 |
| 56 | Q | 100.0 |
| 57 | M | 90.2 |
| 58 | M | 78.7 |
| 59 | A | 16.4 |
| 60 | M | 29.5 |
| 61 | H | 100.0 |
| 62 | N | 78.7 |
| 63 | T | 19.7 |
| 64 | K | 85.2 |
| 65 | G | 100.0 |
| 66 | R | 50.8 |
| 67 | T | 86.9 |
| 68 | W | 100.0 |
| 69 | S | 47.5 |
| 70 | L | 100.0 |
| 71 | M | 95.1 |
| 72 | L | 96.7 |
| 73 | V | 16.4 |
| 74 | S | 90.2 |
| 75 | L | 100.0 |
| 76 | I | 93.4 |
| 77 | I | 16.4 |
| 78 | F | 100.0 |
| 79 | I | 100.0 |
| 80 | A | 27.9 |
| 81 | T | 9.8 |
| 82 | T | 96.7 |
| 83 | N | 100.0 |
| 84 | L | 96.7 |
| 85 | L | 98.4 |
| 86 | G | 100.0 |
| 87 | L | 93.4 |
| 88 | L | 91.8 |
| 89 | P | 100.0 |
| 90 | H | 80.3 |
| 91 | S | 82.0 |
| 92 | F | 100.0 |
| 93 | T | 98.4 |
| 94 | P | 100.0 |
| 95 | T | 100.0 |
| 96 | T | 100.0 |
| 97 | Q | 100.0 |
| 98 | L | 100.0 |
| 99 | S | 96.7 |
| 100 | M | 95.1 |
| 101 | N | 98.4 |
| 102 | L | 83.6 |
| 103 | A | 18.0 |
| 104 | M | 98.4 |
| 105 | A | 98.4 |
| 106 | I | 91.8 |
| 107 | P | 100.0 |
| 108 | L | 100.0 |
| 109 | W | 100.0 |
| 110 | A | 80.3 |
| 111 | G | 85.2 |
| 112 | A | 32.8 |
| 113 | V | 98.4 |
| 114 | I | 55.7 |
| 115 | M | 34.4 |
| 116 | G | 100.0 |
| 117 | F | 90.2 |
| 118 | R | 100.0 |
| 119 | S | 3.3 |
| 120 | K | 100.0 |
| 121 | I | 1.6 |
| 122 | K | 98.4 |
| 123 | N | 14.8 |
| 124 | A | 9.8 |
| 125 | L | 98.4 |
| 126 | A | 96.7 |
| 127 | H | 100.0 |
| 128 | F | 83.6 |
| 129 | L | 98.4 |
| 130 | P | 100.0 |
| 131 | Q | 100.0 |
| 132 | G | 100.0 |
| 133 | T | 100.0 |
| 134 | P | 100.0 |
| 135 | T | 39.3 |
| 136 | P | 68.9 |
| 137 | L | 100.0 |
| 138 | I | 96.7 |
| 139 | P | 100.0 |
| 140 | M | 98.4 |
| 141 | L | 98.4 |
| 142 | V | 49.2 |
| 143 | I | 83.6 |
| 144 | I | 100.0 |
| 145 | E | 98.4 |
| 146 | T | 100.0 |
| 147 | I | 98.4 |
| 148 | S | 100.0 |
| 149 | L | 100.0 |
| 150 | L | 13.1 |
| 151 | I | 100.0 |
| 152 | Q | 98.4 |
| 153 | P | 100.0 |
| 154 | M | 59.0 |
| 155 | A | 98.4 |
| 156 | L | 98.4 |
| 157 | A | 100.0 |
| 158 | V | 95.1 |
| 159 | R | 100.0 |
| 160 | L | 100.0 |
| 161 | T | 100.0 |
| 162 | A | 100.0 |
| 163 | N | 100.0 |
| 164 | I | 100.0 |
| 165 | T | 100.0 |
| 166 | A | 100.0 |
| 167 | G | 100.0 |
| 168 | H | 100.0 |
| 169 | L | 98.4 |
| 170 | L | 100.0 |
| 171 | M | 52.5 |
| 172 | H | 100.0 |
| 173 | L | 100.0 |
| 174 | I | 96.7 |
| 175 | G | 98.4 |
| 176 | S | 24.6 |
| 177 | A | 91.8 |
| 178 | T | 88.5 |
| 179 | L | 98.4 |
| 180 | A | 68.9 |
| 181 | M | 6.6 |
| 182 | S | 23.0 |
| 183 | T | 13.1 |
| 184 | I | 96.7 |
| 185 | N | 23.0 |
| 186 | L | 23.0 |
| 187 | P | 27.9 |
| 188 | S | 8.2 |
| 189 | T | 13.1 |
| 190 | L | 41.0 |
| 191 | I | 85.2 |
| 192 | I | 9.8 |
| 193 | F | 91.8 |
| 194 | T | 36.1 |
| 195 | I | 82.0 |
| 196 | L | 96.7 |
| 197 | I | 42.6 |
| 198 | L | 98.4 |
| 199 | L | 100.0 |
| 200 | T | 96.7 |
| 201 | I | 73.8 |
| 202 | L | 100.0 |
| 203 | E | 100.0 |
| 204 | I | 16.4 |
| 205 | A | 96.7 |
| 206 | V | 98.4 |
| 207 | A | 100.0 |
| 208 | L | 78.7 |
| 209 | I | 100.0 |
| 210 | Q | 100.0 |
| 211 | A | 98.4 |
| 212 | Y | 100.0 |
| 213 | V | 98.4 |
| 214 | F | 100.0 |
| 215 | T | 96.7 |
| 216 | L | 98.4 |
| 217 | L | 100.0 |
| 218 | V | 96.7 |
| 219 | S | 98.4 |
| 220 | L | 98.4 |
| 221 | Y | 100.0 |
| 222 | L | 100.0 |
| 223 | H | 100.0 |
| 224 | D | 91.8 |
| 225 | N | 100.0 |
| 226 | T | 78.7 |

TABLE 2
ATP 8 gene

| AA# | AA | % cons. |
|---|---|---|
| 1 | M | 100.0 |
| 2 | P | 98.4 |
| 3 | Q | 98.4 |
| 4 | L | 98.4 |
| 5 | N | 16.4 |
| 6 | T | 96.7 |
| 7 | T | 11.5 |
| 8 | V | 8.2 |
| 9 | W | 100.0 |
| 10 | P | 11.5 |
| 11 | T | 29.5 |
| 12 | M | 14.8 |
| 13 | I | 95.1 |
| 14 | T | 14.8 |
| 15 | P | 9.8 |
| 16 | M | 85.2 |
| 17 | L | 29.5 |
| 18 | L | 57.4 |
| 19 | T | 70.5 |
| 20 | L | 95.1 |
| 21 | F | 86.9 |
| 22 | L | 16.4 |
| 23 | I | 31.1 |
| 24 | T | 13.1 |
| 25 | Q | 95.1 |
| 26 | L | 86.9 |
| 27 | K | 96.7 |
| 28 | M | 19.7 |
| 29 | L | 21.3 |
| 30 | N | 27.9 |
| 31 | T | 6.6 |
| 32 | N | 16.4 |
| 33 | Y | 62.3 |
| 34 | H | 18.0 |
| 35 | L | 16.4 |
| 36 | P | 16.4 |
| 37 | P | 82.0 |
| 38 | S | 16.4 |
| 39 | P | 42.6 |
| 40 | K | 67.2 |
| 41 | P | 16.4 |
| 42 | M | 18.0 |
| 43 | K | 55.7 |
| 44 | M | 13.1 |
| 45 | K | 14.8 |
| 46 | N | 9.8 |
| 47 | Y | 6.6 |
| 48 | N | 26.2 |
| 49 | K | 11.5 |
| 50 | P | 91.8 |
| 51 | W | 100.0 |
| 52 | E | 77.0 |
| 53 | P | 11.5 |
| 54 | K | 93.4 |
| 55 | W | 100.0 |
| 56 | T | 98.4 |
| 57 | K | 100.0 |
| 58 | I | 77.0 |
| 59 | C | 6.6 |
| 60 | S | 41.0 |
| 61 | L | 9.8 |
| 62 | H | 32.8 |
| 63 | S | 82.0 |
| 64 | L | 78.6 |
| 65 | P | 83.9 |
| 66 | P | 21.4 |
| 67 | Q | 68.6 |
| 68 | S | 68.4 |

TABLE 3
CO1 gene

| AA# | AA | % cons. |
|---|---|---|
| 1 | M | 100.0 |
| 2 | F | 95.1 |
| 3 | A | 8.2 |
| 4 | D | 13.1 |
| 5 | R | 100.0 |
| 6 | W | 100.0 |
| 7 | L | 96.7 |
| 8 | F | 93.4 |
| 9 | S | 100.0 |
| 10 | T | 100.0 |
| 11 | N | 100.0 |
| 12 | H | 100.0 |
| 13 | K | 100.0 |
| 14 | D | 100.0 |
| 15 | I | 100.0 |
| 16 | G | 100.0 |
| 17 | T | 100.0 |
| 18 | L | 100.0 |
| 19 | Y | 100.0 |
| 20 | L | 95.1 |
| 21 | L | 91.8 |
| 22 | F | 100.0 |
| 23 | G | 100.0 |
| 24 | A | 98.4 |
| 25 | W | 100.0 |
| 26 | A | 100.0 |
| 27 | G | 100.0 |
| 28 | V | 13.1 |
| 29 | L | 11.5 |
| 30 | G | 100.0 |
| 31 | T | 98.4 |
| 32 | A | 88.5 |
| 33 | L | 98.4 |
| 34 | S | 100.0 |
| 35 | L | 86.9 |
| 36 | L | 100.0 |
| 37 | I | 100.0 |
| 38 | R | 100.0 |
| 39 | A | 93.4 |
| 40 | E | 100.0 |
| 41 | L | 100.0 |
| 42 | G | 100.0 |
| 43 | Q | 100.0 |
| 44 | P | 100.0 |
| 45 | G | 100.0 |
| 46 | N | 14.8 |
| 47 | L | 100.0 |
| 48 | L | 88.5 |
| 49 | G | 98.4 |
| 50 | N | 14.8 |
| 51 | D | 100.0 |
| 52 | H | 16.4 |
| 53 | I | 93.4 |
| 54 | Y | 100.0 |
| 55 | N | 98.4 |
| 56 | V | 98.4 |
| 57 | I | 75.4 |
| 58 | V | 100.0 |
| 59 | T | 98.4 |
| 60 | A | 98.4 |
| 61 | H | 100.0 |
| 62 | A | 100.0 |
| 63 | F | 100.0 |
| 64 | V | 90.2 |
| 65 | M | 100.0 |
| 66 | I | 100.0 |
| 67 | F | 100.0 |
| 68 | F | 100.0 |
| 69 | M | 98.4 |
| 70 | V | 100.0 |
| 71 | M | 100.0 |
| 72 | P | 100.0 |
| 73 | I | 93.4 |
| 74 | M | 98.4 |
| 75 | I | 93.4 |
| 76 | G | 100.0 |
| 77 | G | 100.0 |
| 78 | F | 100.0 |
| 79 | G | 100.0 |
| 80 | N | 100.0 |
| 81 | W | 100.0 |
| 82 | L | 100.0 |
| 83 | V | 95.1 |
| 84 | P | 100.0 |
| 85 | L | 100.0 |
| 86 | M | 100.0 |
| 87 | I | 100.0 |
| 88 | G | 100.0 |
| 89 | A | 100.0 |
| 90 | P | 100.0 |
| 91 | D | 100.0 |
| 92 | M | 100.0 |
| 93 | A | 100.0 |
| 94 | F | 100.0 |
| 95 | P | 100.0 |
| 96 | R | 100.0 |
| 97 | M | 96.7 |
| 98 | N | 100.0 |
| 99 | N | 100.0 |
| 100 | M | 100.0 |
| 101 | S | 100.0 |
| 102 | F | 98.4 |
| 103 | W | 100.0 |
| 104 | L | 98.4 |
| 105 | L | 100.0 |
| 106 | P | 98.4 |
| 107 | P | 100.0 |
| 108 | S | 100.0 |
| 109 | L | 6.6 |
| 110 | L | 98.4 |
| 111 | L | 100.0 |
| 112 | L | 100.0 |
| 113 | L | 86.9 |
| 114 | A | 91.8 |
| 115 | S | 100.0 |
| 116 | A | 11.5 |
| 117 | M | 72.1 |
| 118 | V | 86.9 |
| 119 | E | 100.0 |
| 120 | A | 100.0 |
| 121 | G | 100.0 |
| 122 | A | 91.8 |
| 123 | G | 100.0 |
| 124 | T | 100.0 |
| 125 | G | 100.0 |
| 126 | W | 100.0 |
| 127 | T | 100.0 |
| 128 | V | 100.0 |
| 129 | Y | 100.0 |
| 130 | P | 100.0 |
| 131 | P | 100.0 |
| 132 | L | 100.0 |
| 133 | A | 98.4 |
| 134 | G | 100.0 |
| 135 | N | 100.0 |
| 136 | Y | 11.5 |
| 137 | S | 16.4 |
| 138 | H | 100.0 |
| 139 | P | 16.4 |
| 140 | G | 100.0 |
| 141 | A | 96.7 |
| 142 | S | 100.0 |
| 143 | V | 100.0 |
| 144 | D | 100.0 |
| 145 | L | 100.0 |
| 146 | T | 73.8 |
| 147 | I | 98.4 |
| 148 | F | 98.4 |
| 149 | S | 100.0 |
| 150 | L | 100.0 |
| 151 | H | 100.0 |
| 152 | L | 100.0 |
| 153 | A | 100.0 |
| 154 | G | 100.0 |
| 155 | V | 68.9 |
| 156 | S | 98.4 |
| 157 | S | 100.0 |
| 158 | I | 100.0 |
| 159 | L | 100.0 |
| 160 | G | 98.4 |
| 161 | A | 96.7 |
| 162 | I | 100.0 |
| 163 | N | 100.0 |
| 164 | F | 100.0 |
| 165 | I | 100.0 |
| 166 | T | 98.4 |
| 167 | T | 100.0 |
| 168 | I | 96.7 |
| 169 | I | 98.4 |
| 170 | N | 100.0 |
| 171 | M | 100.0 |
| 172 | K | 100.0 |
| 173 | P | 98.4 |
| 174 | P | 100.0 |
| 175 | A | 100.0 |
| 176 | M | 78.7 |
| 177 | T | 27.9 |
| 178 | Q | 100.0 |
| 179 | Y | 100.0 |
| 180 | Q | 98.4 |
| 181 | T | 96.7 |
| 182 | P | 100.0 |
| 183 | L | 100.0 |
| 184 | F | 100.0 |
| 185 | V | 100.0 |
| 186 | W | 100.0 |
| 187 | S | 100.0 |
| 188 | V | 86.9 |
| 189 | L | 86.9 |
| 190 | I | 90.2 |
| 191 | T | 100.0 |
| 192 | A | 100.0 |
| 193 | V | 95.1 |
| 194 | L | 100.0 |
| 195 | L | 98.4 |
| 196 | L | 98.4 |
| 197 | L | 100.0 |
| 198 | S | 91.8 |
| 199 | L | 100.0 |
| 200 | P | 100.0 |
| 201 | V | 100.0 |
| 202 | L | 100.0 |
| 203 | A | 100.0 |
| 204 | A | 100.0 |
| 205 | G | 100.0 |
| 206 | I | 100.0 |
| 207 | T | 100.0 |
| 208 | M | 100.0 |
| 209 | L | 100.0 |
| 210 | L | 100.0 |
| 211 | T | 100.0 |
| 212 | D | 100.0 |
| 213 | R | 100.0 |
| 214 | N | 100.0 |
| 215 | L | 100.0 |
| 216 | N | 100.0 |
| 217 | T | 100.0 |
| 218 | T | 100.0 |
| 219 | F | 100.0 |
| 220 | F | 98.4 |
| 221 | D | 98.4 |
| 222 | P | 100.0 |
| 223 | A | 95.1 |
| 224 | G | 100.0 |
| 225 | G | 100.0 |
| 226 | G | 98.4 |
| 227 | D | 100.0 |
| 228 | P | 100.0 |
| 229 | I | 96.7 |
| 230 | L | 100.0 |
| 231 | Y | 100.0 |
| 232 | Q | 100.0 |
| 233 | H | 100.0 |
| 234 | L | 100.0 |
| 235 | F | 100.0 |
| 236 | W | 100.0 |
| 237 | F | 100.0 |
| 238 | F | 100.0 |
| 239 | G | 100.0 |
| 240 | H | 100.0 |
| 241 | P | 100.0 |
| 242 | E | 100.0 |
| 243 | V | 100.0 |
| 244 | Y | 100.0 |
| 245 | I | 100.0 |
| 246 | L | 100.0 |
| 247 | I | 98.4 |
| 248 | L | 98.4 |
| 249 | P | 98.4 |
| 250 | G | 98.4 |
| 251 | F | 100.0 |
| 252 | G | 100.0 |
| 253 | M | 73.8 |
| 254 | I | 96.7 |
| 255 | S | 100.0 |
| 256 | H | 100.0 |
| 257 | I | 93.4 |
| 258 | V | 98.4 |
| 259 | T | 95.1 |
| 260 | Y | 91.8 |
| 261 | Y | 100.0 |
| 262 | S | 100.0 |
| 263 | G | 96.7 |
| 264 | K | 100.0 |
| 265 | K | 98.4 |
| 266 | E | 100.0 |
| 267 | P | 100.0 |
| 268 | F | 100.0 |
| 269 | G | 100.0 |
| 270 | Y | 100.0 |
| 271 | M | 100.0 |
| 272 | G | 100.0 |
| 273 | M | 100.0 |
| 274 | V | 98.4 |
| 275 | W | 98.4 |
| 276 | A | 98.4 |
| 277 | M | 100.0 |
| 278 | M | 90.2 |
| 279 | S | 100.0 |
| 280 | I | 100.0 |
| 281 | G | 100.0 |
| 282 | F | 100.0 |
| 283 | L | 100.0 |
| 284 | G | 100.0 |
| 285 | F | 100.0 |
| 286 | I | 100.0 |
| 287 | V | 100.0 |
| 288 | W | 100.0 |
| 289 | A | 100.0 |
| 290 | H | 100.0 |
| 291 | H | 100.0 |
| 292 | M | 100.0 |
| 293 | F | 100.0 |
| 294 | T | 100.0 |
| 295 | V | 100.0 |
| 296 | G | 100.0 |
| 297 | M | 78.7 |
| 298 | D | 100.0 |
| 299 | V | 100.0 |
| 300 | D | 100.0 |
| 301 | T | 100.0 |
| 302 | R | 100.0 |
| 303 | A | 100.0 |
| 304 | Y | 98.4 |
| 305 | F | 100.0 |
| 306 | T | 100.0 |
| 307 | S | 100.0 |
| 308 | A | 98.4 |
| 309 | T | 100.0 |
| 310 | M | 100.0 |
| 311 | I | 100.0 |
| 312 | I | 100.0 |
| 313 | A | 100.0 |
| 314 | I | 100.0 |
| 315 | P | 100.0 |
| 316 | T | 100.0 |
| 317 | G | 100.0 |
| 318 | V | 100.0 |
| 319 | K | 100.0 |
| 320 | V | 98.4 |
| 321 | F | 100.0 |
| 322 | S | 100.0 |
| 323 | W | 100.0 |
| 324 | L | 100.0 |
| 325 | A | 100.0 |
| 326 | T | 100.0 |
| 327 | L | 100.0 |
| 328 | H | 100.0 |
| 329 | G | 100.0 |
| 330 | S | 11.5 |
| 331 | N | 90.2 |
| 332 | M | 4.9 |
| 333 | K | 96.7 |
| 334 | W | 100.0 |
| 335 | S | 95.1 |
| 336 | A | 11.5 |
| 337 | A | 96.7 |
| 338 | V | 6.6 |
| 339 | L | 78.7 |
| 340 | W | 100.0 |
| 341 | A | 100.0 |
| 342 | L | 100.0 |
| 343 | G | 100.0 |
| 344 | F | 100.0 |
| 345 | I | 100.0 |
| 346 | F | 100.0 |
| 347 | L | 100.0 |
| 348 | F | 100.0 |
| 349 | T | 100.0 |
| 350 | V | 86.9 |
| 351 | G | 100.0 |
| 352 | G | 100.0 |
| 353 | L | 100.0 |
| 354 | T | 100.0 |
| 355 | G | 100.0 |
| 356 | I | 96.7 |
| 357 | V | 93.4 |
| 358 | L | 100.0 |
| 359 | A | 91.8 |
| 360 | N | 100.0 |
| 361 | S | 100.0 |
| 362 | S | 100.0 |
| 363 | L | 100.0 |
| 364 | D | 100.0 |
| 365 | I | 96.7 |
| 366 | V | 95.1 |
| 367 | L | 100.0 |
| 368 | H | 100.0 |
| 369 | D | 100.0 |
| 370 | T | 100.0 |
| 371 | Y | 100.0 |
| 372 | Y | 100.0 |
| 373 | V | 100.0 |
| 374 | V | 98.4 |
| 375 | A | 100.0 |
| 376 | H | 98.4 |
| 377 | F | 100.0 |
| 378 | H | 100.0 |
| 379 | Y | 100.0 |
| 380 | V | 100.0 |
| 381 | L | 100.0 |
| 382 | S | 98.4 |
| 383 | M | 98.4 |
| 384 | G | 100.0 |
| 385 | A | 100.0 |
| 386 | V | 100.0 |
| 387 | F | 100.0 |
| 388 | A | 100.0 |
| 389 | I | 98.4 |
| 390 | M | 93.4 |
| 391 | G | 90.2 |
| 392 | G | 98.4 |
| 393 | F | 98.4 |
| 394 | I | 21.3 |
| 395 | H | 100.0 |
| 396 | W | 100.0 |
| 397 | F | 100.0 |
| 398 | P | 100.0 |
| 399 | L | 100.0 |
| 400 | F | 96.7 |
| 401 | S | 78.7 |
| 402 | G | 100.0 |
| 403 | Y | 93.4 |
| 404 | T | 93.4 |
| 405 | L | 96.7 |
| 406 | D | 32.8 |
| 407 | Q | 32.8 |
| 408 | T | 83.6 |
| 409 | Y | 13.1 |
| 410 | A | 100.0 |
| 411 | K | 100.0 |
| 412 | I | 83.6 |
| 413 | H | 91.8 |
| 414 | F | 100.0 |
| 415 | T | 27.9 |
| 416 | I | 77.0 |
| 417 | M | 98.4 |
| 418 | F | 100.0 |
| 419 | I | 14.8 |
| 420 | G | 100.0 |
| 421 | V | 98.4 |
| 422 | N | 100.0 |
| 423 | L | 52.5 |
| 424 | T | 100.0 |
| 425 | F | 100.0 |
| 426 | F | 100.0 |
| 427 | P | 100.0 |
| 428 | Q | 100.0 |
| 429 | H | 98.4 |
| 430 | F | 100.0 |
| 431 | L | 100.0 |
| 432 | G | 100.0 |
| 433 | L | 100.0 |
| 434 | S | 91.8 |
| 435 | G | 100.0 |
| 436 | M | 100.0 |
| 437 | P | 100.0 |
| 438 | R | 100.0 |
| 439 | R | 98.4 |
| 440 | Y | 100.0 |
| 441 | S | 100.0 |
| 442 | D | 100.0 |
| 443 | Y | 100.0 |
| 444 | P | 100.0 |
| 445 | D | 100.0 |
| 446 | A | 100.0 |
| 447 | Y | 100.0 |
| 448 | T | 100.0 |
| 449 | T | 70.5 |
| 450 | W | 100.0 |
| 451 | N | 100.0 |
| 452 | I | 16.4 |
| 453 | L | 18.0 |
| 454 | S | 100.0 |
| 455 | S | 100.0 |
| 456 | V | 13.1 |
| 457 | G | 100.0 |
| 458 | S | 100.0 |
| 459 | F | 98.4 |
| 460 | I | 98.4 |
| 461 | S | 100.0 |
| 462 | L | 100.0 |
| 463 | T | 96.7 |
| 464 | A | 100.0 |
| 465 | V | 96.7 |
| 466 | M | 63.9 |
| 467 | L | 78.7 |
| 468 | M | 98.4 |
| 469 | I | 57.4 |
| 470 | F | 96.7 |
| 471 | M | 75.4 |
| 472 | I | 80.3 |
| 473 | W | 100.0 |
| 474 | E | 100.0 |
| 475 | A | 98.4 |
| 476 | F | 100.0 |
| 477 | A | 91.8 |
| 478 | S | 96.7 |
| 479 | K | 100.0 |
| 480 | R | 100.0 |
| 481 | K | 16.4 |
| 482 | V | 96.7 |
| 483 | L | 41.0 |
| 484 | M | 16.4 |
| 485 | V | 75.4 |
| 486 | E | 82.0 |
| 487 | E | 6.6 |
| 488 | P | 19.7 |
| 489 | S | 42.6 |
| 490 | M | 1.6 |
| 491 | N | 95.1 |
| 492 | L | 72.1 |
| 493 | E | 100.0 |
| 494 | W | 100.0 |
| 495 | L | 98.4 |
| 496 | Y | 24.6 |
| 497 | G | 100.0 |
| 498 | C | 96.7 |
| 499 | P | 100.0 |
| 500 | P | 100.0 |
| 501 | P | 98.4 |
| 502 | Y | 93.4 |
| 503 | H | 100.0 |
| 504 | T | 100.0 |
| 505 | F | 100.0 |
| 506 | E | 100.0 |
| 507 | E | 90.2 |
| 508 | P | 96.7 |
| 509 | V | 31.1 |
| 510 | Y | 80.3 |
| 511 | M | 13.1 |
| 512 | K | 54.1 |
| 513 | S | 13.1 |

TABLE 4
CO2 gene

| AA# | AA | % cons. |
|---|---|---|
| 1 | M | 100.0 |
| 2 | A | 85.2 |
| 3 | H | 19.7 |
| 4 | A | 11.5 |
| 5 | A | 13.1 |
| 6 | Q | 90.2 |
| 7 | V | 11.5 |
| 8 | G | 98.4 |
| 9 | L | 39.3 |
| 10 | Q | 100.0 |
| 11 | D | 98.4 |
| 12 | A | 100.0 |
| 13 | T | 82.0 |
| 14 | S | 100.0 |
| 15 | P | 100.0 |
| 16 | I | 95.1 |
| 17 | M | 100.0 |
| 18 | E | 100.0 |
| 19 | E | 100.0 |
| 20 | L | 100.0 |
| 21 | I | 13.1 |
| 22 | T | 4.9 |
| 23 | F | 100.0 |
| 24 | H | 98.4 |
| 25 | D | 100.0 |
| 26 | H | 98.4 |
| 27 | A | 27.9 |
| 28 | L | 98.4 |
| 29 | M | 100.0 |
| 30 | I | 95.1 |
| 31 | I | 14.8 |
| 32 | F | 98.4 |
| 33 | L | 96.7 |
| 34 | I | 100.0 |
| 35 | C | 9.8 |
| 36 | F | 14.8 |
| 37 | L | 100.0 |
| 38 | V | 98.4 |
| 39 | L | 100.0 |
| 40 | Y | 100.0 |
| 41 | A | 14.8 |
| 42 | L | 14.8 |
| 43 | F | 11.5 |
| 44 | L | 70.5 |
| 45 | T | 14.8 |
| 46 | L | 100.0 |
| 47 | T | 95.1 |
| 48 | T | 96.7 |
| 49 | K | 90.2 |
| 50 | L | 100.0 |
| 51 | T | 95.1 |
| 52 | N | 14.8 |
| 53 | T | 96.7 |
| 54 | N | 21.3 |
| 55 | I | 14.8 |
| 56 | S | 9.8 |
| 57 | D | 93.4 |
| 58 | A | 98.4 |
| 59 | Q | 100.0 |
| 60 | E | 96.7 |
| 61 | M | 16.4 |
| 62 | E | 100.0 |
| 63 | T | 98.4 |
| 64 | V | 27.9 |
| 65 | W | 100.0 |
| 66 | T | 100.0 |
| 67 | I | 95.1 |
| 68 | L | 96.7 |
| 69 | P | 98.4 |
| 70 | A | 100.0 |
| 71 | I | 73.8 |
| 72 | I | 100.0 |
| 73 | L | 100.0 |
| 74 | V | 16.4 |
| 75 | L | 83.6 |
| 76 | I | 100.0 |
| 77 | A | 100.0 |
| 78 | L | 100.0 |
| 79 | P | 100.0 |
| 80 | S | 100.0 |
| 81 | L | 100.0 |
| 82 | R | 98.4 |
| 83 | I | 98.4 |
| 84 | L | 100.0 |
| 85 | Y | 100.0 |
| 86 | M | 90.2 |
| 87 | T | 16.4 |
| 88 | D | 100.0 |
| 89 | E | 100.0 |
| 90 | V | 8.2 |
| 91 | N | 91.8 |
| 92 | D | 18.0 |
| 93 | P | 100.0 |
| 94 | S | 63.9 |
| 95 | L | 78.7 |
| 96 | T | 100.0 |
| 97 | I | 27.9 |
| 98 | K | 100.0 |
| 99 | S | 13.1 |
| 100 | I | 18.0 |
| 101 | G | 100.0 |
| 102 | H | 100.0 |
| 103 | Q | 100.0 |
| 104 | W | 100.0 |
| 105 | Y | 98.4 |
| 106 | W | 100.0 |
| 107 | T | 13.1 |
| 108 | Y | 100.0 |
| 109 | E | 98.4 |
| 110 | Y | 98.4 |
| 111 | T | 91.8 |
| 112 | D | 98.4 |
| 113 | Y | 98.4 |
| 114 | G | 14.8 |
| 115 | G | 14.8 |
| 116 | L | 100.0 |
| 117 | I | 14.8 |
| 118 | F | 98.4 |
| 119 | N | 14.8 |
| 120 | S | 100.0 |
| 121 | Y | 100.0 |
| 122 | M | 98.4 |
| 123 | L | 14.8 |
| 124 | P | 96.7 |
| 125 | P | 14.8 |
| 126 | L | 14.8 |
| 127 | F | 14.8 |
| 128 | L | 100.0 |
| 129 | E | 14.8 |
| 130 | P | 93.4 |
| 131 | G | 100.0 |
| 132 | D | 27.9 |
| 133 | L | 86.9 |
| 134 | R | 98.4 |
| 135 | L | 100.0 |
| 136 | L | 100.0 |
| 137 | D | 9.8 |
| 138 | V | 98.4 |
| 139 | D | 100.0 |
| 140 | N | 98.4 |
| 141 | R | 96.7 |
| 142 | V | 80.3 |
| 143 | V | 93.4 |
| 144 | L | 98.4 |
| 145 | P | 98.4 |
| 146 | I | 9.8 |
| 147 | E | 93.4 |
| 148 | A | 16.4 |
| 149 | P | 47.5 |
| 150 | I | 73.8 |
| 151 | R | 100.0 |
| 152 | M | 86.9 |
| 153 | M | 14.8 |
| 154 | I | 86.9 |
| 155 | T | 18.0 |
| 156 | S | 100.0 |
| 157 | Q | 16.4 |
| 158 | D | 100.0 |
| 159 | V | 100.0 |
| 160 | L | 98.4 |
| 161 | H | 98.4 |
| 162 | S | 91.8 |
| 163 | W | 100.0 |
| 164 | A | 82.0 |
| 165 | V | 85.2 |
| 166 | P | 100.0 |
| 167 | T | 9.8 |
| 168 | L | 93.4 |
| 169 | G | 100.0 |
| 170 | L | 91.8 |
| 171 | K | 100.0 |
| 172 | T | 90.2 |
| 173 | D | 100.0 |
| 174 | A | 100.0 |
| 175 | I | 93.4 |
| 176 | P | 100.0 |
| 177 | G | 100.0 |
| 178 | R | 100.0 |
| 179 | L | 100.0 |
| 180 | N | 100.0 |
| 181 | Q | 100.0 |
| 182 | T | 57.4 |
| 183 | T | 95.1 |
| 184 | F | 14.8 |
| 185 | T | 36.1 |
| 186 | A | 32.8 |
| 187 | T | 70.5 |
| 188 | R | 100.0 |
| 189 | P | 100.0 |
| 190 | G | 100.0 |
| 191 | V | 24.6 |
| 192 | Y | 63.9 |
| 193 | Y | 100.0 |
| 194 | G | 100.0 |
| 195 | Q | 100.0 |
| 196 | C | 100.0 |
| 197 | S | 100.0 |
| 198 | E | 100.0 |
| 199 | I | 100.0 |
| 200 | C | 100.0 |
| 201 | G | 100.0 |
| 202 | A | 21.3 |
| 203 | N | 100.0 |
| 204 | H | 100.0 |
| 205 | S | 100.0 |
| 206 | F | 100.0 |
| 207 | M | 100.0 |
| 208 | P | 100.0 |
| 209 | I | 100.0 |
| 210 | V | 96.7 |
| 211 | L | 93.4 |
| 212 | E | 100.0 |
| 213 | L | 60.7 |
| 214 | I | 16.4 |
| 215 | P | 88.5 |
| 216 | L | 96.7 |
| 217 | K | 75.4 |
| 218 | I | 16.4 |
| 219 | F | 100.0 |
| 220 | E | 100.0 |
| 221 | M | 14.8 |
| 222 | G | 14.8 |
| 223 | P | 14.8 |
| 224 | V | 16.4 |
| 225 | F | 14.8 |
| 226 | T | 11.5 |
| 227 | L | 65.0 |

TABLE 5
CO3 gene

| AA# | AA | % cons. |
|---|---|---|
| 1 | M | 100.0 |
| 2 | T | 93.4 |
| 3 | H | 100.0 |
| 4 | Q | 100.0 |
| 5 | S | 11.5 |
| 6 | H | 100.0 |
| 7 | A | 93.4 |
| 8 | Y | 100.0 |
| 9 | H | 100.0 |
| 10 | M | 98.4 |
| 11 | V | 98.4 |
| 12 | K | 14.8 |
| 13 | P | 100.0 |
| 14 | S | 100.0 |
| 15 | P | 100.0 |
| 16 | W | 100.0 |
| 17 | P | 100.0 |
| 18 | L | 100.0 |
| 19 | T | 100.0 |
| 20 | G | 100.0 |
| 21 | A | 100.0 |
| 22 | L | 93.4 |
| 23 | S | 100.0 |
| 24 | A | 100.0 |
| 25 | L | 95.1 |
| 26 | L | 98.4 |
| 27 | M | 54.1 |
| 28 | T | 98.4 |
| 29 | S | 100.0 |
| 30 | G | 100.0 |
| 31 | L | 95.1 |
| 32 | A | 23.0 |
| 33 | M | 98.4 |
| 34 | W | 100.0 |
| 35 | F | 100.0 |
| 36 | H | 100.0 |
| 37 | F | 77.0 |
| 38 | H | 14.8 |
| 39 | S | 91.8 |
| 40 | M | 21.3 |
| 41 | T | 34.4 |
| 42 | L | 96.7 |
| 43 | L | 90.2 |
| 44 | M | 16.4 |
| 45 | L | 72.1 |
| 46 | G | 96.7 |
| 47 | L | 88.5 |
| 48 | L | 49.2 |
| 49 | T | 82.0 |
| 50 | N | 90.2 |
| 51 | T | 23.0 |
| 52 | L | 95.1 |
| 53 | T | 96.7 |
| 54 | M | 93.4 |
| 55 | Y | 88.5 |
| 56 | Q | 100.0 |
| 57 | W | 100.0 |
| 58 | W | 100.0 |
| 59 | R | 100.0 |
| 60 | D | 100.0 |
| 61 | V | 27.9 |
| 62 | T | 1.6 |
| 63 | R | 100.0 |
| 64 | E | 100.0 |
| 65 | S | 54.1 |
| 66 | T | 100.0 |
| 67 | Y | 27.9 |
| 68 | Q | 100.0 |
| 69 | G | 100.0 |
| 70 | H | 100.0 |
| 71 | H | 100.0 |
| 72 | T | 100.0 |
| 73 | P | 70.5 |
| 74 | P | 16.4 |
| 75 | V | 98.4 |
| 76 | Q | 100.0 |
| 77 | K | 98.4 |
| 78 | G | 95.1 |
| 79 | L | 100.0 |
| 80 | R | 100.0 |
| 81 | Y | 100.0 |
| 82 | G | 100.0 |
| 83 | M | 100.0 |
| 84 | I | 73.8 |
| 85 | L | 100.0 |
| 86 | F | 100.0 |
| 87 | I | 100.0 |
| 88 | T | 14.8 |
| 89 | S | 100.0 |
| 90 | E | 100.0 |
| 91 | V | 91.8 |
| 92 | F | 82.0 |
| 93 | F | 100.0 |
| 94 | F | 100.0 |
| 95 | A | 54.1 |
| 96 | G | 100.0 |
| 97 | F | 100.0 |
| 98 | F | 100.0 |
| 99 | W | 100.0 |
| 100 | A | 98.4 |
| 101 | F | 100.0 |
| 102 | Y | 100.0 |
| 103 | H | 100.0 |
| 104 | S | 100.0 |
| 105 | S | 100.0 |
| 106 | L | 100.0 |
| 107 | A | 93.4 |
| 108 | P | 100.0 |
| 109 | T | 100.0 |
| 110 | P | 88.5 |
| 111 | Q | 13.1 |
| 112 | L | 98.4 |
| 113 | G | 100.0 |
| 114 | G | 90.2 |
| 115 | H | 18.0 |
| 116 | W | 100.0 |
| 117 | P | 100.0 |
| 118 | P | 98.4 |
| 119 | T | 82.0 |
| 120 | G | 98.4 |
| 121 | I | 96.7 |
| 122 | T | 31.1 |
| 123 | P | 96.7 |
| 124 | L | 98.4 |
| 125 | N | 95.1 |
| 126 | P | 98.4 |
| 127 | L | 83.6 |
| 128 | E | 93.4 |
| 129 | V | 100.0 |
| 130 | P | 100.0 |
| 131 | L | 100.0 |
| 132 | L | 100.0 |
| 133 | N | 100.0 |
| 134 | T | 100.0 |
| 135 | S | 88.5 |
| 136 | V | 83.6 |
| 137 | L | 100.0 |
| 138 | L | 100.0 |
| 139 | A | 100.0 |
| 140 | S | 100.0 |
| 141 | G | 98.4 |
| 142 | V | 100.0 |
| 143 | S | 88.5 |
| 144 | I | 98.4 |
| 145 | T | 100.0 |
| 146 | W | 100.0 |
| 147 | A | 96.7 |
| 148 | H | 100.0 |
| 149 | H | 98.4 |
| 150 | S | 100.0 |
| 151 | L | 98.4 |
| 152 | M | 98.4 |
| 153 | E | 95.1 |
| 154 | N | 13.1 |
| 155 | N | 82.0 |
| 156 | R | 98.4 |
| 157 | N | 16.4 |
| 158 | Q | 39.3 |
| 159 | M | 91.8 |
| 160 | I | 39.3 |
| 161 | Q | 96.7 |
| 162 | A | 95.1 |
| 163 | L | 100.0 |
| 164 | L | 31.1 |
| 165 | I | 91.8 |
| 166 | T | 100.0 |
| 167 | I | 100.0 |
| 168 | L | 31.1 |
| 169 | L | 100.0 |
| 170 | G | 100.0 |
| 171 | L | 36.1 |
| 172 | Y | 100.0 |
| 173 | F | 100.0 |
| 174 | T | 100.0 |
| 175 | L | 72.1 |
| 176 | L | 98.4 |
| 177 | Q | 100.0 |
| 178 | A | 88.5 |
| 179 | S | 82.0 |
| 180 | E | 100.0 |
| 181 | Y | 100.0 |
| 182 | F | 21.3 |
| 183 | E | 100.0 |
| 184 | S | 14.8 |
| 185 | P | 54.1 |
| 186 | F | 100.0 |
| 187 | T | 90.2 |
| 188 | I | 100.0 |
| 189 | S | 91.8 |
| 190 | D | 100.0 |
| 191 | G | 95.1 |
| 192 | I | 44.3 |
| 193 | Y | 100.0 |
| 194 | G | 100.0 |
| 195 | S | 100.0 |
| 196 | T | 100.0 |
| 197 | F | 100.0 |
| 198 | F | 100.0 |
| 199 | V | 54.1 |
| 200 | A | 95.1 |
| 201 | T | 100.0 |
| 202 | G | 100.0 |
| 203 | F | 100.0 |
| 204 | H | 100.0 |
| 205 | G | 100.0 |
| 206 | L | 95.1 |
| 207 | H | 100.0 |
| 208 | V | 100.0 |
| 209 | I | 100.0 |
| 210 | I | 98.4 |
| 211 | G | 100.0 |
| 212 | S | 88.5 |
| 213 | T | 91.8 |
| 214 | F | 98.4 |
| 215 | L | 100.0 |
| 216 | T | 27.9 |
| 217 | I | 13.1 |
| 218 | C | 100.0 |
| 219 | F | 50.8 |
| 220 | I | 19.7 |
| 221 | R | 98.4 |
| 222 | Q | 100.0 |
| 223 | L | 78.7 |
| 224 | M | 8.2 |
| 225 | F | 70.5 |
| 226 | H | 100.0 |
| 227 | F | 100.0 |
| 228 | T | 100.0 |
| 229 | S | 93.4 |
| 230 | K | 27.9 |
| 231 | H | 100.0 |
| 232 | H | 100.0 |
| 233 | F | 100.0 |
| 234 | G | 100.0 |
| 235 | F | 100.0 |
| 236 | E | 98.4 |
| 237 | A | 96.7 |
| 238 | A | 98.4 |
| 239 | A | 98.4 |
| 240 | W | 100.0 |
| 241 | Y | 100.0 |
| 242 | W | 100.0 |
| 243 | H | 98.4 |
| 244 | F | 98.4 |
| 245 | V | 100.0 |
| 246 | D | 100.0 |
| 247 | V | 100.0 |
| 248 | V | 95.1 |
| 249 | W | 100.0 |
| 250 | L | 100.0 |
| 251 | F | 98.4 |
| 252 | L | 100.0 |
| 253 | Y | 100.0 |
| 254 | V | 91.8 |
| 255 | S | 100.0 |
| 256 | I | 100.0 |
| 257 | Y | 100.0 |
| 258 | W | 100.0 |
| 259 | W | 100.0 |
| 260 | G | 100.0 |
| 261 | S | 100.0 |

TABLE 6
CytB gene

| AA # | AA | % cons. |
|---|---|---|
| 1 | M | 100.0 |
| 2 | T | 85.2 |
| 3 | P | 13.1 |
| 4 | M | 16.4 |
| 5 | R | 100.0 |
| 6 | K | 100.0 |
| 7 | I | 4.9 |
| 8 | N | 13.1 |
| 9 | P | 100.0 |
| 10 | L | 95.1 |
| 11 | M | 45.9 |
| 12 | K | 100.0 |
| 13 | L | 11.5 |
| 14 | I | 77.0 |
| 15 | N | 100.0 |
| 16 | H | 50.8 |
| 17 | S | 80.3 |
| 18 | F | 85.2 |
| 19 | I | 95.1 |
| 20 | D | 100.0 |
| 21 | L | 100.0 |
| 22 | P | 100.0 |
| 23 | T | 55.7 |
| 24 | P | 100.0 |
| 25 | S | 93.4 |
| 26 | N | 91.8 |
| 27 | I | 96.7 |
| 28 | S | 100.0 |
| 29 | A | 37.7 |
| 30 | W | 100.0 |
| 31 | W | 100.0 |
| 32 | N | 98.4 |
| 33 | F | 100.0 |
| 34 | G | 100.0 |
| 35 | S | 100.0 |
| 36 | L | 100.0 |
| 37 | L | 100.0 |
| 38 | G | 95.1 |
| 39 | A | 23.0 |
| 40 | C | 100.0 |
| 41 | L | 100.0 |
| 42 | I | 62.3 |
| 43 | L | 47.5 |
| 44 | Q | 100.0 |
| 45 | I | 98.4 |
| 46 | T | 9.8 |
| 47 | T | 100.0 |
| 48 | G | 100.0 |
| 49 | L | 100.0 |
| 50 | F | 100.0 |
| 51 | L | 100.0 |
| 52 | A | 100.0 |
| 53 | M | 100.0 |
| 54 | H | 100.0 |
| 55 | Y | 100.0 |
| 56 | S | 14.8 |
| 57 | P | 29.5 |
| 58 | D | 100.0 |
| 59 | A | 11.5 |
| 60 | S | 21.3 |
| 61 | T | 95.1 |
| 62 | A | 100.0 |
| 63 | F | 100.0 |
| 64 | S | 93.4 |
| 65 | S | 100.0 |
| 66 | I | 19.7 |
| 67 | A | 37.7 |
| 68 | H | 100.0 |
| 69 | I | 98.4 |
| 70 | T | 16.4 |
| 71 | R | 100.0 |
| 72 | D | 98.4 |
| 73 | V | 98.4 |
| 74 | N | 95.1 |
| 75 | Y | 98.4 |
| 76 | G | 100.0 |
| 77 | W | 100.0 |
| 78 | I | 37.7 |
| 79 | I | 91.8 |
| 80 | R | 100.0 |
| 81 | Y | 83.6 |
| 82 | L | 73.8 |
| 83 | H | 98.4 |
| 84 | A | 98.4 |
| 85 | N | 100.0 |
| 86 | G | 100.0 |
| 87 | A | 100.0 |
| 88 | S | 100.0 |
| 89 | M | 86.9 |
| 90 | F | 93.4 |
| 91 | F | 100.0 |
| 92 | I | 77.0 |
| 93 | C | 98.4 |
| 94 | L | 96.7 |
| 95 | F | 70.5 |
| 96 | L | 57.4 |
| 97 | H | 100.0 |
| 98 | I | 27.9 |
| 99 | G | 98.4 |
| 100 | R | 100.0 |
| 101 | G | 98.4 |
| 102 | L | 68.9 |
| 103 | Y | 100.0 |
| 104 | Y | 100.0 |
| 105 | G | 100.0 |
| 106 | S | 100.0 |
| 107 | F | 14.8 |
| 108 | L | 32.8 |
| 109 | Y | 29.5 |
| 110 | S | 16.4 |
| 111 | E | 95.1 |
| 112 | T | 100.0 |
| 113 | W | 100.0 |
| 114 | N | 100.0 |
| 115 | I | 88.5 |
| 116 | G | 100.0 |
| 117 | I | 59.0 |
| 118 | I | 70.5 |
| 119 | L | 100.0 |
| 120 | L | 100.0 |
| 121 | L | 29.5 |
| 122 | A | 29.5 |
| 123 | T | 27.9 |
| 124 | M | 100.0 |
| 125 | A | 93.4 |
| 126 | T | 98.4 |
| 127 | A | 100.0 |
| 128 | F | 100.0 |
| 129 | M | 83.6 |
| 130 | G | 100.0 |
| 131 | Y | 100.0 |
| 132 | V | 100.0 |
| 133 | L | 100.0 |
| 134 | P | 100.0 |
| 135 | W | 100.0 |
| 136 | G | 100.0 |
| 137 | Q | 100.0 |
| 138 | M | 100.0 |
| 139 | S | 100.0 |
| 140 | F | 100.0 |
| 141 | W | 100.0 |
| 142 | G | 98.4 |
| 143 | A | 100.0 |
| 144 | T | 100.0 |
| 145 | V | 100.0 |
| 146 | I | 100.0 |
| 147 | T | 100.0 |
| 148 | N | 100.0 |
| 149 | L | 100.0 |
| 150 | L | 98.4 |
| 151 | S | 100.0 |
| 152 | A | 100.0 |
| 153 | I | 95.1 |
| 154 | P | 100.0 |
| 155 | Y | 100.0 |
| 156 | I | 90.2 |
| 157 | G | 100.0 |
| 158 | T | 88.5 |
| 159 | D | 41.0 |
| 160 | L | 100.0 |
| 161 | V | 100.0 |
| 162 | Q | 26.2 |
| 163 | W | 100.0 |
| 164 | I | 80.3 |
| 165 | W | 100.0 |
| 166 | G | 100.0 |
| 167 | G | 98.4 |
| 168 | Y | 13.1 |
| 169 | S | 96.7 |
| 170 | V | 96.7 |
| 171 | D | 95.1 |
| 172 | S | 11.5 |
| 173 | P | 14.8 |
| 174 | T | 100.0 |
| 175 | L | 100.0 |
| 176 | T | 96.7 |
| 177 | R | 98.4 |
| 178 | F | 100.0 |
| 179 | F | 100.0 |
| 180 | T | 18.0 |
| 181 | F | 85.2 |
| 182 | H | 100.0 |
| 183 | F | 100.0 |
| 184 | I | 90.2 |
| 185 | L | 96.7 |
| 186 | P | 100.0 |
| 187 | F | 100.0 |
| 188 | I | 85.2 |
| 189 | I | 78.7 |
| 190 | A | 24.6 |
| 191 | A | 95.1 |
| 192 | L | 91.8 |
| 193 | A | 44.3 |
| 194 | A | 13.1 |
| 195 | L | 11.5 |
| 196 | H | 100.0 |
| 197 | L | 100.0 |
| 198 | L | 93.4 |
| 199 | F | 100.0 |
| 200 | L | 100.0 |
| 201 | H | 100.0 |
| 202 | E | 100.0 |
| 203 | T | 95.1 |
| 204 | G | 100.0 |
| 205 | S | 100.0 |
| 206 | N | 96.7 |
| 207 | N | 100.0 |
| 208 | P | 100.0 |
| 209 | L | 16.4 |
| 210 | G | 100.0 |
| 211 | I | 68.9 |
| 212 | T | 11.5 |
| 213 | S | 88.5 |
| 214 | H | 9.8 |
| 215 | S | 52.5 |
| 216 | D | 100.0 |
| 217 | K | 91.8 |
| 218 | I | 100.0 |
| 219 | T | 11.5 |
| 220 | F | 100.0 |
| 221 | H | 100.0 |
| 222 | P | 100.0 |
| 223 | Y | 100.0 |
| 224 | Y | 93.4 |
| 225 | T | 91.8 |
| 226 | I | 83.6 |
| 227 | K | 100.0 |
| 228 | D | 100.0 |
| 229 | A | 9.8 |
| 230 | L | 90.2 |
| 231 | G | 100.0 |
| 232 | L | 39.3 |
| 233 | L | 59.0 |
| 234 | L | 63.9 |
| 235 | F | 11.5 |
| 236 | L | 32.8 |
| 237 | L | 77.0 |
| 238 | S | 3.3 |
| 239 | L | 96.7 |
| 240 | M | 36.1 |
| 241 | T | 42.6 |
| 242 | L | 100.0 |
| 243 | T | 29.5 |
| 244 | L | 96.7 |
| 245 | F | 95.1 |
| 246 | S | 65.6 |
| 247 | P | 100.0 |
| 248 | D | 96.7 |
| 249 | L | 80.3 |
| 250 | L | 100.0 |
| 251 | G | 90.2 |
| 252 | D | 100.0 |
| 253 | P | 100.0 |
| 254 | D | 96.7 |
| 255 | N | 100.0 |
| 256 | Y | 88.5 |
| 257 | T | 73.8 |
| 258 | L | 11.5 |
| 259 | A | 98.4 |
| 260 | N | 95.1 |
| 261 | P | 100.0 |
| 262 | L | 100.0 |
| 263 | N | 55.7 |
| 264 | T | 100.0 |
| 265 | P | 100.0 |
| 266 | P | 93.4 |
| 267 | H | 100.0 |
| 268 | I | 100.0 |
| 269 | K | 100.0 |
| 270 | P | 100.0 |
| 271 | E | 100.0 |
| 272 | W | 100.0 |
| 273 | Y | 100.0 |
| 274 | F | 100.0 |
| 275 | L | 100.0 |
| 276 | F | 98.4 |
| 277 | A | 96.7 |
| 278 | Y | 100.0 |
| 279 | T | 8.2 |
| 280 | I | 100.0 |
| 281 | L | 100.0 |
| 282 | R | 100.0 |
| 283 | S | 100.0 |
| 284 | V | 16.4 |
| 285 | P | 100.0 |
| 286 | N | 100.0 |
| 287 | K | 100.0 |
| 288 | L | 100.0 |
| 289 | G | 100.0 |
| 290 | G | 100.0 |
| 291 | V | 100.0 |
| 292 | L | 91.8 |
| 293 | A | 100.0 |
| 294 | L | 100.0 |
| 295 | L | 26.2 |
| 296 | L | 60.7 |
| 297 | S | 100.0 |
| 298 | I | 100.0 |
| 299 | L | 95.1 |
| 300 | I | 83.6 |
| 301 | L | 100.0 |
| 302 | A | 60.7 |
| 303 | M | 4.9 |
| 304 | I | 60.7 |
| 305 | P | 100.0 |
| 306 | I | 9.8 |
| 307 | L | 96.7 |
| 308 | H | 98.4 |
| 309 | M | 11.5 |
| 310 | S | 88.5 |
| 311 | K | 91.8 |
| 312 | Q | 96.7 |
| 313 | Q | 16.4 |
| 314 | S | 85.2 |
| 315 | M | 80.3 |
| 316 | M | 75.4 |
| 317 | F | 98.4 |
| 318 | R | 100.0 |
| 319 | P | 100.0 |
| 320 | L | 60.7 |
| 321 | S | 90.2 |
| 322 | Q | 100.0 |
| 323 | S | 1.6 |
| 324 | L | 82.0 |
| 325 | Y | 16.4 |
| 326 | W | 100.0 |
| 327 | L | 45.9 |
| 328 | L | 100.0 |
| 329 | A | 14.8 |
| 330 | A | 83.6 |
| 331 | D | 73.8 |
| 332 | L | 100.0 |
| 333 | L | 73.8 |
| 334 | I | 23.0 |
| 335 | L | 100.0 |
| 336 | T | 100.0 |
| 337 | W | 100.0 |
| 338 | I | 100.0 |
| 339 | G | 100.0 |
| 340 | G | 93.4 |
| 341 | Q | 98.4 |
| 342 | P | 100.0 |
| 343 | V | 100.0 |
| 344 | S | 11.5 |
| 345 | Y | 18.0 |
| 346 | P | 100.0 |
| 347 | F | 65.6 |
| 348 | T | 4.9 |
| 349 | I | 65.6 |
| 350 | I | 93.4 |
| 351 | G | 98.4 |
| 352 | Q | 100.0 |
| 353 | V | 29.5 |
| 354 | A | 100.0 |
| 355 | S | 100.0 |
| 356 | V | 14.8 |
| 357 | L | 73.8 |
| 358 | Y | 100.0 |
| 359 | F | 100.0 |
| 360 | T | 32.8 |
| 361 | T | 20.0 |
| 362 | I | 86.9 |
| 363 | L | 83.6 |
| 364 | I | 39.3 |
| 365 | L | 86.9 |
| 366 | M | 85.2 |
| 367 | P | 100.0 |
| 368 | T | 9.8 |
| 369 | I | 6.6 |
| 370 | S | 52.5 |
| 371 | L | 50.8 |
| 372 | I | 63.9 |
| 373 | E | 100.0 |
| 374 | N | 96.7 |
| 375 | K | 39.3 |
| 376 | M | 31.1 |
| 377 | L | 85.2 |
| 378 | K | 91.8 |
| 379 | W | 0 |
| 380 | A | 0 |

TABLE 7
ND1 gene.

| AA# | AA | % cons. |
|---|---|---|
| 1 | M | 95.0 |
| 2 | P | 10.0 |
| 3 | M | 51.7 |
| 4 | A | 6.6 |
| 5 | N | 100.0 |
| 6 | L | 52.5 |
| 7 | L | 83.6 |
| 8 | L | 37.7 |
| 9 | L | 82.0 |
| 10 | I | 78.7 |
| 11 | V | 36.1 |
| 12 | P | 93.4 |
| 13 | I | 75.4 |
| 14 | L | 95.1 |
| 15 | I | 18.0 |
| 16 | A | 100.0 |
| 17 | M | 37.7 |
| 18 | A | 98.4 |
| 19 | F | 98.4 |
| 20 | L | 100.0 |
| 21 | M | 11.5 |
| 22 | L | 100.0 |
| 23 | T | 14.8 |
| 24 | E | 98.4 |
| 25 | R | 98.4 |
| 26 | K | 100.0 |
| 27 | I | 36.1 |
| 28 | L | 100.0 |
| 29 | G | 100.0 |
| 30 | Y | 96.7 |
| 31 | M | 95.1 |
| 32 | Q | 100.0 |
| 33 | L | 80.3 |
| 34 | R | 100.0 |
| 35 | K | 100.0 |
| 36 | G | 100.0 |
| 37 | P | 100.0 |
| 38 | N | 100.0 |
| 39 | V | 32.8 |
| 40 | V | 93.4 |
| 41 | G | 100.0 |
| 42 | P | 95.1 |
| 43 | Y | 83.6 |
| 44 | G | 100.0 |
| 45 | L | 88.5 |
| 46 | L | 98.4 |
| 47 | Q | 100.0 |
| 48 | P | 100.0 |
| 49 | F | 41.0 |
| 50 | A | 100.0 |
| 51 | D | 100.0 |
| 52 | A | 100.0 |
| 53 | M | 27.9 |
| 54 | K | 100.0 |
| 55 | L | 100.0 |
| 56 | F | 98.4 |
| 57 | T | 65.6 |
| 58 | K | 100.0 |
| 59 | E | 100.0 |
| 60 | P | 100.0 |
| 61 | L | 91.8 |
| 62 | K | 18.0 |
| 63 | P | 100.0 |
| 64 | A | 14.8 |
| 65 | T | 95.1 |
| 66 | S | 95.1 |
| 67 | T | 16.4 |
| 68 | I | 29.5 |
| 69 | T | 36.1 |
| 70 | L | 42.6 |
| 71 | Y | 19.7 |
| 72 | I | 78.7 |
| 73 | T | 9.8 |
| 74 | A | 96.7 |
| 75 | P | 100.0 |
| 76 | T | 31.1 |
| 77 | L | 93.4 |
| 78 | A | 93.4 |
| 79 | L | 95.1 |
| 80 | T | 73.8 |
| 81 | I | 19.7 |
| 82 | A | 98.4 |
| 83 | L | 93.4 |
| 84 | L | 16.4 |
| 85 | L | 26.2 |
| 86 | W | 100.0 |
| 87 | T | 23.0 |
| 88 | P | 100.0 |
| 89 | L | 85.2 |
| 90 | P | 98.4 |
| 91 | M | 98.4 |
| 92 | P | 100.0 |
| 93 | N | 14.8 |
| 94 | P | 85.2 |
| 95 | L | 100.0 |
| 96 | V | 24.6 |
| 97 | N | 86.9 |
| 98 | L | 39.3 |
| 99 | N | 98.4 |
| 100 | L | 88.5 |
| 101 | G | 86.9 |
| 102 | L | 36.1 |
| 103 | L | 96.7 |
| 104 | F | 100.0 |
| 105 | I | 54.1 |
| 106 | L | 100.0 |
| 107 | A | 95.1 |
| 108 | T | 27.9 |
| 109 | S | 100.0 |
| 110 | S | 88.5 |
| 111 | L | 100.0 |
| 112 | A | 67.2 |
| 113 | V | 100.0 |
| 114 | Y | 96.7 |
| 115 | S | 96.7 |
| 116 | I | 96.7 |
| 117 | L | 100.0 |
| 118 | W | 100.0 |
| 119 | S | 100.0 |
| 120 | G | 100.0 |
| 121 | W | 100.0 |
| 122 | A | 98.4 |
| 123 | S | 100.0 |
| 124 | N | 100.0 |
| 125 | S | 100.0 |
| 126 | N | 19.7 |
| 127 | Y | 100.0 |
| 128 | A | 93.4 |
| 129 | L | 98.4 |
| 130 | I | 85.2 |
| 131 | G | 100.0 |
| 132 | A | 96.7 |
| 133 | L | 96.7 |
| 134 | R | 100.0 |
| 135 | A | 100.0 |
| 136 | V | 100.0 |
| 137 | A | 100.0 |
| 138 | Q | 100.0 |
| 139 | T | 98.4 |
| 140 | I | 98.4 |
| 141 | S | 100.0 |
| 142 | Y | 100.0 |
| 143 | E | 100.0 |
| 144 | V | 95.1 |
| 145 | T | 91.8 |
| 146 | L | 90.2 |
| 147 | A | 93.4 |
| 148 | I | 93.4 |
| 149 | I | 98.4 |
| 150 | L | 95.1 |
| 151 | L | 100.0 |
| 152 | S | 88.5 |
| 153 | T | 13.1 |
| 154 | L | 80.3 |
| 155 | L | 93.4 |
| 156 | M | 70.5 |
| 157 | S | 34.4 |
| 158 | G | 100.0 |
| 159 | S | 100.0 |
| 160 | F | 91.8 |
| 161 | N | 16.4 |
| 162 | L | 98.4 |
| 163 | S | 67.2 |
| 164 | T | 65.6 |
| 165 | L | 95.1 |
| 166 | I | 75.4 |
| 167 | T | 54.1 |
| 168 | T | 98.4 |
| 169 | Q | 100.0 |
| 170 | E | 93.4 |
| 171 | H | 45.9 |
| 172 | L | 39.3 |
| 173 | W | 100.0 |
| 174 | L | 91.8 |
| 175 | L | 36.1 |
| 176 | L | 39.3 |
| 177 | P | 80.3 |
| 178 | S | 37.7 |
| 179 | W | 100.0 |
| 180 | P | 100.0 |
| 181 | L | 95.1 |
| 182 | A | 80.3 |
| 183 | M | 93.4 |
| 184 | M | 98.4 |
| 185 | W | 100.0 |
| 186 | F | 86.9 |
| 187 | I | 88.5 |
| 188 | S | 100.0 |
| 189 | T | 100.0 |
| 190 | L | 100.0 |
| 191 | A | 100.0 |
| 192 | E | 100.0 |
| 193 | T | 100.0 |
| 194 | N | 100.0 |
| 195 | R | 100.0 |
| 196 | T | 11.5 |
| 197 | P | 98.4 |
| 198 | F | 98.4 |
| 199 | D | 100.0 |
| 200 | L | 100.0 |
| 201 | A | 11.5 |
| 202 | E | 98.4 |
| 203 | G | 100.0 |
| 204 | E | 100.0 |
| 205 | S | 100.0 |
| 206 | E | 100.0 |
| 207 | L | 100.0 |
| 208 | V | 100.0 |
| 209 | S | 100.0 |
| 210 | G | 100.0 |
| 211 | F | 100.0 |
| 212 | N | 98.4 |
| 213 | I | 11.5 |
| 214 | E | 100.0 |
| 215 | Y | 100.0 |
| 216 | A | 96.7 |
| 217 | A | 91.8 |
| 218 | G | 100.0 |
| 219 | P | 98.4 |
| 220 | F | 98.4 |
| 221 | A | 100.0 |
| 222 | L | 80.3 |
| 223 | F | 100.0 |
| 224 | F | 98.4 |
| 225 | M | 50.8 |
| 226 | A | 98.4 |
| 227 | E | 100.0 |
| 228 | Y | 100.0 |
| 229 | T | 24.6 |
| 230 | N | 100.0 |
| 231 | I | 100.0 |
| 232 | I | 91.8 |
| 233 | M | 75.4 |
| 234 | M | 100.0 |
| 235 | N | 100.0 |
| 236 | T | 13.1 |
| 237 | L | 72.1 |
| 238 | T | 96.7 |
| 239 | T | 59.0 |
| 240 | T | 27.9 |
| 241 | I | 26.2 |
| 242 | F | 100.0 |
| 243 | L | 63.9 |
| 244 | G | 88.5 |
| 245 | T | 31.1 |
| 246 | T | 13.1 |
| 247 | Y | 19.7 |
| 248 | D | 11.5 |
| 249 | A | 4.9 |
| 250 | L | 8.2 |
| 251 | S | 14.8 |
| 252 | P | 88.5 |
| 253 | E | 86.9 |
| 254 | L | 77.0 |
| 255 | Y | 59.0 |
| 256 | T | 82.0 |
| 257 | T | 31.1 |
| 258 | Y | 4.9 |
| 259 | F | 85.2 |
| 260 | V | 11.5 |
| 261 | T | 24.6 |
| 262 | K | 95.1 |
| 263 | T | 80.3 |
| 264 | L | 85.2 |
| 265 | L | 68.9 |
| 266 | L | 96.7 |
| 267 | T | 93.4 |
| 268 | S | 24.6 |
| 269 | L | 36.1 |
| 270 | F | 100.0 |
| 271 | L | 100.0 |
| 272 | W | 100.0 |
| 273 | I | 80.3 |
| 274 | R | 100.0 |
| 275 | T | 9.8 |
| 276 | A | 9.8 |
| 277 | Y | 100.0 |
| 278 | P | 100.0 |
| 279 | R | 100.0 |
| 280 | F | 98.4 |
| 281 | R | 98.4 |
| 282 | Y | 100.0 |
| 283 | D | 100.0 |
| 284 | Q | 98.4 |
| 285 | L | 100.0 |
| 286 | M | 98.4 |
| 287 | H | 91.8 |
| 288 | L | 98.4 |
| 289 | L | 100.0 |
| 290 | W | 100.0 |
| 291 | K | 100.0 |
| 292 | N | 85.2 |
| 293 | F | 100.0 |
| 294 | L | 100.0 |
| 295 | P | 100.0 |
| 296 | L | 90.2 |
| 297 | T | 98.4 |
| 298 | L | 100.0 |
| 299 | A | 93.4 |
| 300 | L | 77.0 |
| 301 | L | 14.8 |
| 302 | M | 88.5 |
| 303 | W | 93.4 |
| 304 | Y | 27.9 |
| 305 | V | 44.3 |
| 306 | S | 77.0 |
| 307 | M | 29.5 |
| 308 | P | 95.1 |
| 309 | I | 77.0 |
| 310 | T | 23.0 |
| 311 | I | 11.5 |
| 312 | S | 49.2 |
| 313 | S | 50.8 |
| 314 | I | 80.3 |
| 315 | P | 100.0 |
| 316 | P | 100.0 |
| 317 | Q | 86.9 |
| 318 | T | 67.2 |

TABLE 8
ND2 gene

| AA# | AA | % cons. |
|---|---|---|
| 1 | M | 100.0 |
| 2 | N | 75.4 |
| 3 | P | 96.7 |
| 4 | L | 44.3 |
| 5 | A | 19.7 |
| 6 | Q | 14.8 |
| 7 | P | 13.1 |
| 8 | V | 1.6 |
| 9 | I | 83.6 |
| 10 | Y | 31.1 |
| 11 | S | 26.2 |
| 12 | T | 85.2 |
| 13 | I | 29.5 |
| 14 | F | 26.2 |
| 15 | A | 11.5 |
| 16 | G | 98.4 |
| 17 | T | 93.4 |
| 18 | L | 29.5 |
| 19 | I | 78.7 |
| 20 | T | 52.5 |
| 21 | A | 13.1 |
| 22 | L | 29.5 |
| 23 | S | 98.4 |
| 24 | S | 86.9 |
| 25 | H | 93.4 |
| 26 | W | 95.1 |
| 27 | F | 24.6 |
| 28 | F | 13.1 |
| 29 | T | 16.4 |
| 30 | W | 100.0 |
| 31 | V | 23.0 |
| 32 | G | 100.0 |
| 33 | L | 54.1 |
| 34 | E | 100.0 |
| 35 | M | 85.2 |
| 36 | N | 86.9 |
| 37 | M | 68.9 |
| 38 | L | 85.2 |
| 39 | A | 93.4 |
| 40 | F | 19.7 |
| 41 | I | 95.1 |
| 42 | P | 100.0 |
| 43 | V | 14.8 |
| 44 | L | 67.2 |
| 45 | T | 31.1 |
| 46 | K | 67.2 |
| 47 | K | 55.7 |
| 48 | M | 9.8 |
| 49 | N | 59.0 |
| 50 | P | 91.8 |
| 51 | R | 98.4 |
| 52 | S | 62.3 |
| 53 | T | 67.2 |
| 54 | E | 100.0 |
| 55 | A | 88.5 |
| 56 | A | 67.2 |
| 57 | I | 24.6 |
| 58 | K | 100.0 |
| 59 | Y | 98.4 |
| 60 | F | 96.7 |
| 61 | L | 85.2 |
| 62 | T | 85.2 |
| 63 | Q | 100.0 |
| 64 | A | 88.5 |
| 65 | T | 90.2 |
| 66 | A | 100.0 |
| 67 | S | 100.0 |
| 68 | M | 93.4 |
| 69 | I | 41.0 |
| 70 | L | 85.2 |
| 71 | L | 24.6 |
| 72 | M | 59.0 |
| 73 | A | 73.8 |
| 74 | I | 67.2 |
| 75 | L | 14.8 |
| 76 | F | 3.3 |
| 77 | N | 96.7 |
| 78 | N | 14.8 |
| 79 | M | 41.0 |
| 80 | L | 21.3 |
| 81 | S | 77.0 |
| 82 | G | 88.5 |
| 83 | Q | 75.4 |
| 84 | W | 100.0 |
| 85 | T | 77.0 |
| 86 | M | 9.8 |
| 87 | T | 39.3 |
| 88 | N | 26.7 |
| 89 | T | 13.1 |
| 90 | T | 26.2 |
| 91 | N | 90.2 |
| 92 | Q | 24.6 |
| 93 | Y | 11.5 |
| 94 | S | 23.0 |
| 95 | S | 85.2 |
| 96 | L | 23.0 |
| 97 | M | 41.0 |
| 98 | I | 21.3 |
| 99 | M | 6.6 |
| 100 | M | 19.7 |
| 101 | A | 93.4 |
| 102 | M | 18.0 |
| 103 | A | 55.7 |
| 104 | M | 83.6 |
| 105 | K | 100.0 |
| 106 | L | 96.7 |
| 107 | G | 100.0 |
| 108 | M | 34.4 |
| 109 | A | 67.2 |
| 110 | P | 96.7 |
| 111 | F | 98.4 |
| 112 | H | 98.4 |
| 113 | F | 88.5 |
| 114 | W | 98.4 |
| 115 | V | 88.5 |
| 116 | P | 100.0 |
| 117 | E | 100.0 |
| 118 | V | 100.0 |
| 119 | T | 88.5 |
| 120 | Q | 100.0 |
| 121 | G | 100.0 |
| 122 | T | 18.0 |
| 123 | P | 59.0 |
| 124 | L | 98.4 |
| 125 | T | 27.9 |
| 126 | S | 75.4 |
| 127 | G | 93.4 |
| 128 | L | 60.7 |
| 129 | L | 26.2 |
| 130 | L | 93.4 |
| 131 | L | 100.0 |
| 132 | T | 100.0 |
| 133 | W | 100.0 |
| 134 | Q | 100.0 |
| 135 | K | 100.0 |
| 136 | L | 62.3 |
| 137 | A | 98.4 |
| 138 | P | 100.0 |
| 139 | I | 21.3 |
| 140 | S | 86.9 |
| 141 | I | 73.8 |
| 142 | M | 23.0 |
| 143 | Y | 83.6 |
| 144 | Q | 95.1 |
| 145 | I | 82.0 |
| 146 | S | 55.7 |
| 147 | P | 65.6 |
| 148 | S | 73.8 |
| 149 | L | 29.5 |
| 150 | N | 91.8 |
| 151 | V | 9.8 |
| 152 | S | 6.6 |
| 153 | L | 57.4 |
| 154 | L | 60.7 |
| 155 | L | 54.1 |
| 156 | T | 55.7 |
| 157 | L | 37.7 |
| 158 | S | 37.7 |
| 159 | I | 44.3 |
| 160 | L | 63.9 |
| 161 | S | 100.0 |
| 162 | I | 70.5 |
| 163 | M | 16.4 |
| 164 | A | 6.6 |
| 165 | G | 100.0 |
| 166 | S | 14.8 |
| 167 | W | 100.0 |
| 168 | G | 95.1 |
| 169 | G | 100.0 |
| 170 | L | 100.0 |
| 171 | N | 100.0 |
| 172 | Q | 100.0 |
| 173 | T | 98.4 |
| 174 | Q | 93.4 |
| 175 | L | 95.1 |
| 176 | R | 96.7 |
| 177 | K | 100.0 |
| 178 | I | 93.4 |
| 179 | L | 47.5 |
| 180 | A | 96.7 |
| 181 | Y | 96.7 |
| 182 | S | 98.4 |
| 183 | S | 100.0 |
| 184 | I | 100.0 |
| 185 | T | 16.4 |
| 186 | H | 98.4 |
| 187 | M | 95.1 |
| 188 | G | 100.0 |
| 189 | W | 100.0 |
| 190 | M | 100.0 |
| 191 | M | 21.3 |
| 192 | A | 67.2 |
| 193 | V | 27.9 |
| 194 | L | 63.9 |
| 195 | P | 27.9 |
| 196 | Y | 83.6 |
| 197 | N | 88.5 |
| 198 | P | 90.2 |
| 199 | N | 14.8 |
| 200 | M | 32.8 |
| 201 | T | 62.3 |
| 202 | I | 42.6 |
| 203 | L | 93.4 |
| 204 | N | 93.4 |
| 205 | L | 91.8 |
| 206 | T | 31.1 |
| 207 | I | 83.6 |
| 208 | Y | 100.0 |
| 209 | I | 82.0 |
| 210 | I | 26.2 |
| 211 | L | 37.7 |
| 212 | T | 100.0 |
| 213 | T | 19.7 |
| 214 | T | 57.4 |
| 215 | A | 9.8 |
| 216 | F | 98.4 |
| 217 | L | 21.3 |
| 218 | L | 47.5 |
| 219 | L | 45.9 |
| 220 | N | 21.3 |
| 221 | L | 36.1 |
| 222 | N | 60.7 |
| 223 | S | 70.5 |
| 224 | S | 49.2 |
| 225 | T | 96.7 |
| 226 | T | 73.8 |
| 227 | T | 75.4 |
| 228 | L | 68.9 |
| 229 | L | 9.8 |
| 230 | L | 93.4 |
| 231 | S | 85.2 |
| 232 | R | 13.1 |
| 233 | T | 59.0 |
| 234 | W | 100.0 |
| 235 | N | 98.4 |
| 236 | K | 88.5 |
| 237 | L | 21.3 |
| 238 | T | 23.0 |
| 239 | W | 13.1 |
| 240 | L | 27.9 |
| 241 | T | 63.9 |
| 242 | P | 18.0 |
| 243 | L | 52.5 |
| 244 | I | 59.0 |
| 245 | P | 13.1 |
| 246 | S | 13.1 |
| 247 | T | 54.1 |
| 248 | L | 75.4 |
| 249 | L | 83.6 |
| 250 | S | 100.0 |
| 251 | L | 77.0 |
| 252 | G | 100.0 |
| 253 | G | 100.0 |
| 254 | L | 100.0 |
| 255 | P | 100.0 |
| 256 | P | 100.0 |
| 257 | L | 98.4 |
| 258 | T | 52.5 |
| 259 | G | 98.4 |
| 260 | F | 98.4 |
| 261 | L | 31.1 |
| 262 | P | 98.4 |
| 263 | K | 98.4 |
| 264 | W | 98.4 |
| 265 | A | 14.8 |
| 266 | I | 90.2 |
| 267 | I | 85.2 |
| 268 | E | 11.5 |
| 269 | E | 100.0 |
| 270 | F | 9.8 |
| 271 | T | 65.6 |
| 272 | K | 82.0 |
| 273 | N | 88.5 |
| 274 | N | 42.6 |
| 275 | S | 37.7 |
| 276 | L | 24.6 |
| 277 | I | 72.1 |
| 278 | I | 13.1 |
| 279 | P | 77.0 |
| 280 | T | 72.1 |
| 281 | I | 16.4 |
| 282 | M | 91.8 |
| 283 | A | 91.8 |
| 284 | T | 8.2 |
| 285 | I | 11.5 |
| 286 | T | 19.7 |
| 287 | L | 100.0 |
| 288 | L | 91.8 |
| 289 | N | 98.4 |
| 290 | L | 98.4 |
| 291 | Y | 73.8 |
| 292 | F | 100.0 |
| 293 | Y | 100.0 |
| 294 | L | 13.1 |
| 295 | R | 100.0 |
| 296 | L | 86.9 |
| 297 | I | 50.8 |
| 298 | Y | 100.0 |
| 299 | S | 72.1 |
| 300 | T | 67.2 |
| 301 | S | 60.7 |
| 302 | I | 11.5 |
| 303 | T | 100.0 |
| 304 | L | 29.5 |
| 305 | L | 14.8 |
| 306 | P | 98.4 |
| 307 | M | 9.8 |
| 308 | S | 24.6 |
| 309 | N | 95.1 |
| 310 | N | 93.4 |
| 311 | V | 11.5 |
| 312 | K | 100.0 |
| 313 | M | 72.1 |
| 314 | K | 72.1 |
| 315 | W | 93.4 |
| 316 | Q | 63.9 |
| 317 | F | 68.9 |
| 318 | E | 50.0 |
| 319 | H | 13.1 |
| 320 | T | 55.9 |
| 321 | K | 86.9 |
| 322 | P | 21.7 |
| 323 | T | 26.2 |
| 324 | P | 31.1 |
| 325 | F | 21.3 |
| 326 | L | 68.9 |
| 327 | P | 77.0 |
| 328 | T | 44.3 |
| 329 | L | 72.1 |
| 330 | I | 54.1 |
| 331 | A | 1.7 |
| 332 | L | 45.9 |
| 333 | T | 13.1 |
| 334 | T | 73.8 |
| 335 | L | 50.8 |
| 336 | L | 70.5 |
| 337 | L | 98.4 |
| 338 | P | 100.0 |
| 339 | I | 16.4 |
| 340 | S | 32.8 |
| 341 | P | 98.4 |
| 342 | F | 8.2 |
| 343 | M | 21.3 |
| 344 | L | 23.3 |
| 345 | M | 11.9 |
| 346 | I | 12.1 |
| 347 | L | 12.3 |

TABLE 9
ND3 gene

| AA# | AA | % cons. |
|---|---|---|
| 1 | M | 91.8 |
| 2 | N | 98.4 |
| 3 | F | 13.1 |
| 4 | A | 14.8 |
| 5 | L | 60.7 |
| 6 | I | 9.8 |
| 7 | L | 88.5 |
| 8 | M | 11.5 |
| 9 | I | 32.8 |
| 10 | N | 98.4 |
| 11 | T | 60.7 |
| 12 | L | 44.3 |
| 13 | L | 95.1 |
| 14 | A | 67.2 |
| 15 | L | 29.5 |
| 16 | L | 65.6 |
| 17 | L | 90.2 |
| 18 | M | 13.1 |
| 19 | I | 16.4 |
| 20 | I | 85.2 |
| 21 | T | 13.1 |
| 22 | F | 100.0 |
| 23 | W | 98.4 |
| 24 | L | 91.8 |
| 25 | P | 100.0 |
| 26 | Q | 96.7 |
| 27 | L | 78.7 |
| 28 | N | 82.0 |
| 29 | G | 1.6 |
| 30 | Y | 98.4 |
| 31 | M | 16.4 |
| 32 | E | 98.4 |
| 33 | K | 100.0 |
| 34 | S | 32.8 |
| 35 | T | 6.6 |
| 36 | P | 98.4 |
| 37 | Y | 100.0 |
| 38 | E | 100.0 |
| 39 | C | 100.0 |
| 40 | G | 100.0 |
| 41 | F | 100.0 |
| 42 | D | 100.0 |
| 43 | P | 100.0 |
| 44 | M | 59.0 |
| 45 | S | 14.8 |
| 46 | P | 14.8 |
| 47 | A | 98.4 |
| 48 | R | 96.7 |
| 49 | V | 4.9 |
| 50 | P | 100.0 |
| 51 | F | 100.0 |
| 52 | S | 98.4 |
| 53 | M | 95.1 |
| 54 | K | 100.0 |
| 55 | F | 100.0 |
| 56 | F | 100.0 |
| 57 | L | 100.0 |
| 58 | V | 85.2 |
| 59 | A | 100.0 |
| 60 | I | 100.0 |
| 61 | T | 100.0 |
| 62 | F | 100.0 |
| 63 | L | 100.0 |
| 64 | L | 98.4 |
| 65 | F | 98.4 |
| 66 | D | 98.4 |
| 67 | L | 100.0 |
| 68 | E | 100.0 |
| 69 | I | 96.7 |
| 70 | A | 100.0 |
| 71 | L | 96.7 |
| 72 | L | 100.0 |
| 73 | L | 100.0 |
| 74 | P | 96.7 |
| 75 | L | 96.7 |
| 76 | P | 100.0 |
| 77 | W | 100.0 |
| 78 | A | 98.4 |
| 79 | L | 14.8 |
| 80 | Q | 95.1 |
| 81 | T | 67.2 |
| 82 | T | 24.6 |
| 83 | N | 73.8 |
| 84 | L | 73.8 |
| 85 | P | 14.8 |
| 86 | L | 36.1 |
| 87 | M | 80.3 |
| 88 | V | 6.6 |
| 89 | M | 9.8 |
| 90 | S | 11.5 |
| 91 | S | 31.1 |
| 92 | L | 75.4 |
| 93 | L | 27.9 |
| 94 | L | 98.4 |
| 95 | I | 85.2 |
| 96 | I | 16.4 |
| 97 | I | 34.4 |
| 98 | L | 100.0 |
| 99 | A | 73.8 |
| 100 | L | 47.5 |
| 101 | S | 59.0 |
| 102 | L | 100.0 |
| 103 | A | 95.1 |
| 104 | Y | 100.0 |
| 105 | E | 100.0 |
| 106 | W | 100.0 |
| 107 | L | 24.6 |
| 108 | Q | 83.6 |
| 109 | K | 88.5 |
| 110 | G | 100.0 |
| 111 | L | 100.0 |
| 112 | D | 16.4 |
| 113 | W | 100.0 |
| 114 | T | 44.3 |
| 115 | E | 96.6 |

TABLE 10
ND4 gene

| AA # | AA | % cons. |
|---|---|---|
| 1 | M | 100.0 |
| 2 | L | 100.0 |
| 3 | K | 100.0 |
| 4 | L | 18.0 |
| 5 | I | 85.2 |
| 6 | V | 4.9 |
| 7 | P | 98.4 |
| 8 | T | 93.4 |
| 9 | I | 50.8 |
| 10 | M | 96.7 |
| 11 | L | 98.4 |
| 12 | L | 31.1 |
| 13 | P | 100.0 |
| 14 | L | 80.3 |
| 15 | T | 88.5 |
| 16 | W | 91.8 |
| 17 | L | 62.3 |
| 18 | S | 98.4 |
| 19 | K | 78.7 |
| 20 | K | 27.9 |
| 21 | H | 11.5 |
| 22 | M | 60.7 |
| 23 | I | 67.2 |
| 24 | W | 100.0 |
| 25 | I | 85.2 |
| 26 | N | 98.4 |
| 27 | T | 54.1 |
| 28 | T | 88.5 |
| 29 | T | 32.8 |
| 30 | H | 50.8 |
| 31 | S | 96.7 |
| 32 | L | 80.3 |
| 33 | I | 18.0 |
| 34 | I | 96.7 |
| 35 | S | 86.9 |
| 36 | I | 9.8 |
| 37 | I | 55.7 |
| 38 | P | 14.8 |
| 39 | L | 93.4 |
| 40 | L | 29.5 |
| 41 | F | 21.3 |
| 42 | F | 13.1 |
| 43 | N | 65.6 |
| 44 | Q | 82.0 |
| 45 | I | 6.6 |
| 46 | N | 37.7 |
| 47 | N | 18.3 |
| 48 | N | 60.7 |
| 49 | L | 15.0 |
| 50 | F | 8.2 |
| 51 | S | 19.7 |
| 52 | C | 4.9 |
| 53 | S | 88.5 |
| 54 | P | 8.2 |
| 55 | T | 23.0 |
| 56 | F | 90.2 |
| 57 | S | 31.1 |
| 58 | S | 68.9 |
| 59 | D | 100.0 |
| 60 | P | 45.9 |
| 61 | L | 98.4 |
| 62 | T | 13.1 |
| 63 | T | 55.7 |
| 64 | P | 100.0 |
| 65 | L | 100.0 |
| 66 | L | 90.2 |
| 67 | M | 19.7 |
| 68 | L | 100.0 |
| 69 | T | 85.2 |
| 70 | T | 55.7 |
| 71 | W | 100.0 |
| 72 | L | 100.0 |
| 73 | L | 98.4 |
| 74 | P | 100.0 |
| 75 | L | 100.0 |
| 76 | T | 8.2 |
| 77 | I | 44.3 |
| 78 | M | 60.7 |
| 79 | A | 100.0 |
| 80 | S | 98.4 |
| 81 | Q | 100.0 |
| 82 | R | 6.6 |
| 83 | H | 100.0 |
| 84 | L | 91.8 |
| 85 | S | 49.2 |
| 86 | S | 4.9 |
| 87 | E | 95.1 |
| 88 | P | 42.6 |
| 89 | L | 47.5 |
| 90 | S | 14.8 |
| 91 | R | 82.0 |
| 92 | K | 96.7 |
| 93 | K | 96.7 |
| 94 | L | 77.0 |
| 95 | Y | 88.5 |
| 96 | L | 27.9 |
| 97 | S | 36.1 |
| 98 | M | 83.6 |
| 99 | L | 88.5 |
| 100 | I | 77.0 |
| 101 | S | 26.2 |
| 102 | L | 100.0 |
| 103 | Q | 98.4 |
| 104 | I | 34.4 |
| 105 | S | 24.6 |
| 106 | L | 100.0 |
| 107 | I | 88.5 |
| 108 | M | 95.1 |
| 109 | T | 86.9 |
| 110 | F | 100.0 |
| 111 | T | 47.5 |
| 112 | A | 91.8 |
| 113 | T | 67.2 |
| 114 | E | 98.4 |
| 115 | L | 88.5 |
| 116 | I | 90.2 |
| 117 | M | 44.3 |
| 118 | F | 91.8 |
| 119 | Y | 98.4 |
| 120 | I | 93.4 |
| 121 | F | 6.6 |
| 122 | F | 100.0 |
| 123 | E | 100.0 |
| 124 | T | 27.9 |
| 125 | T | 100.0 |
| 126 | L | 100.0 |
| 127 | I | 73.8 |
| 128 | P | 100.0 |
| 129 | T | 100.0 |
| 130 | L | 96.7 |
| 131 | A | 4.9 |
| 132 | I | 90.2 |
| 133 | I | 100.0 |
| 134 | T | 96.7 |
| 135 | R | 96.7 |
| 136 | W | 100.0 |
| 137 | G | 100.0 |
| 138 | N | 90.2 |
| 139 | Q | 100.0 |
| 140 | P | 16.4 |
| 141 | E | 98.4 |
| 142 | R | 100.0 |
| 143 | L | 96.7 |
| 144 | N | 98.4 |
| 145 | A | 100.0 |
| 146 | G | 98.4 |
| 147 | T | 21.3 |
| 148 | Y | 100.0 |
| 149 | F | 100.0 |
| 150 | L | 100.0 |
| 151 | F | 100.0 |
| 152 | Y | 100.0 |
| 153 | T | 100.0 |
| 154 | L | 100.0 |
| 155 | V | 41.0 |
| 156 | G | 98.4 |
| 157 | S | 100.0 |
| 158 | L | 93.4 |
| 159 | P | 100.0 |
| 160 | L | 100.0 |
| 161 | L | 100.0 |
| 162 | I | 42.6 |
| 163 | A | 83.6 |
| 164 | L | 100.0 |
| 165 | I | 37.7 |
| 166 | Y | 60.7 |
| 167 | T | 26.2 |
| 168 | H | 19.7 |
| 169 | N | 85.2 |
| 170 | T | 34.4 |
| 171 | L | 36.1 |
| 172 | G | 98.4 |
| 173 | S | 70.5 |
| 174 | L | 96.7 |
| 175 | N | 86.9 |
| 176 | I | 27.9 |
| 177 | L | 73.8 |
| 178 | L | 50.8 |
| 179 | L | 37.7 |
| 180 | T | 26.2 |
| 181 | L | 39.3 |
| 182 | T | 24.6 |
| 183 | A | 26.2 |
| 184 | Q | 45.9 |
| 185 | E | 11.5 |
| 186 | L | 73.8 |
| 187 | S | 14.8 |
| 188 | N | 32.8 |
| 189 | S | 65.6 |
| 190 | W | 91.8 |
| 191 | A | 8.2 |
| 192 | N | 73.8 |
| 193 | N | 27.9 |
| 194 | L | 42.6 |
| 195 | M | 29.5 |
| 196 | W | 100.0 |
| 197 | L | 90.2 |
| 198 | A | 91.8 |
| 199 | Y | 13.1 |
| 200 | T | 14.8 |
| 201 | M | 88.5 |
| 202 | A | 98.4 |
| 203 | F | 100.0 |
| 204 | M | 83.6 |
| 205 | V | 88.5 |
| 206 | K | 100.0 |
| 207 | M | 96.7 |
| 208 | P | 100.0 |
| 209 | L | 95.1 |
| 210 | Y | 100.0 |
| 211 | G | 100.0 |
| 212 | L | 83.6 |
| 213 | H | 96.7 |
| 214 | L | 100.0 |
| 215 | W | 100.0 |
| 216 | L | 100.0 |
| 217 | P | 100.0 |
| 218 | K | 100.0 |
| 219 | A | 100.0 |
| 220 | H | 100.0 |
| 221 | V | 100.0 |
| 222 | E | 100.0 |
| 223 | A | 100.0 |
| 224 | P | 100.0 |
| 225 | I | 100.0 |
| 226 | A | 100.0 |
| 227 | G | 100.0 |
| 228 | S | 100.0 |
| 229 | M | 100.0 |
| 230 | V | 90.2 |
| 231 | L | 100.0 |
| 232 | A | 100.0 |
| 233 | A | 100.0 |
| 234 | V | 45.9 |
| 235 | L | 100.0 |
| 236 | L | 100.0 |
| 237 | K | 100.0 |
| 238 | L | 100.0 |
| 239 | G | 100.0 |
| 240 | G | 96.7 |
| 241 | Y | 100.0 |
| 242 | G | 100.0 |
| 243 | M | 88.5 |
| 244 | M | 68.9 |
| 245 | R | 100.0 |
| 246 | L | 18.0 |
| 247 | T | 75.4 |
| 248 | L | 16.4 |
| 249 | I | 41.0 |
| 250 | L | 88.5 |
| 251 | N | 42.6 |
| 252 | P | 100.0 |
| 253 | L | 54.1 |
| 254 | T | 96.7 |
| 255 | K | 23.0 |
| 256 | H | 27.9 |
| 257 | M | 88.5 |
| 258 | A | 77.0 |
| 259 | Y | 100.0 |
| 260 | P | 100.0 |
| 261 | F | 100.0 |
| 262 | L | 49.2 |
| 263 | V | 4.9 |
| 264 | L | 98.4 |
| 265 | S | 96.7 |
| 266 | L | 86.9 |
| 267 | W | 100.0 |
| 268 | G | 100.0 |
| 269 | M | 100.0 |
| 270 | I | 80.3 |
| 271 | M | 100.0 |
| 272 | T | 98.4 |
| 273 | S | 98.4 |
| 274 | S | 93.4 |
| 275 | I | 93.4 |
| 276 | C | 96.7 |
| 277 | L | 98.4 |
| 278 | R | 100.0 |
| 279 | Q | 100.0 |
| 280 | T | 96.7 |
| 281 | D | 100.0 |
| 282 | L | 96.7 |
| 283 | K | 100.0 |
| 284 | S | 100.0 |
| 285 | L | 98.4 |
| 286 | I | 100.0 |
| 287 | A | 100.0 |
| 288 | Y | 100.0 |
| 289 | S | 98.4 |
| 290 | S | 100.0 |
| 291 | I | 8.2 |
| 292 | S | 100.0 |
| 293 | H | 100.0 |
| 294 | M | 100.0 |
| 295 | A | 88.5 |
| 296 | L | 100.0 |
| 297 | V | 98.4 |
| 298 | V | 13.1 |
| 299 | T | 13.1 |
| 300 | A | 98.4 |
| 301 | I | 67.2 |
| 302 | L | 82.0 |
| 303 | I | 88.5 |
| 304 | Q | 100.0 |
| 305 | T | 90.2 |
| 306 | P | 91.8 |
| 307 | W | 86.9 |
| 308 | S | 96.7 |
| 309 | F | 57.4 |
| 310 | T | 13.1 |
| 311 | G | 100.0 |
| 312 | A | 100.0 |
| 313 | V | 4.9 |
| 314 | I | 9.8 |
| 315 | L | 96.7 |
| 316 | M | 100.0 |
| 317 | I | 83.6 |
| 318 | A | 100.0 |
| 319 | H | 100.0 |
| 320 | G | 98.4 |
| 321 | L | 98.4 |
| 322 | T | 100.0 |
| 323 | S | 100.0 |
| 324 | S | 98.4 |
| 325 | L | 32.8 |
| 326 | L | 91.8 |
| 327 | F | 98.4 |
| 328 | C | 96.7 |
| 329 | L | 100.0 |
| 330 | A | 100.0 |
| 331 | N | 100.0 |
| 332 | S | 68.9 |
| 333 | N | 100.0 |
| 334 | Y | 100.0 |
| 335 | E | 100.0 |
| 336 | R | 98.4 |
| 337 | T | 39.3 |
| 338 | H | 98.4 |
| 339 | S | 96.7 |
| 340 | R | 98.4 |
| 341 | I | 16.4 |
| 342 | M | 96.7 |
| 343 | I | 82.0 |
| 344 | L | 93.4 |
| 345 | S | 14.8 |
| 346 | Q | 9.8 |
| 347 | G | 100.0 |
| 348 | L | 100.0 |
| 349 | Q | 98.4 |
| 350 | T | 59.0 |
| 351 | L | 55.7 |
| 352 | L | 93.4 |
| 353 | P | 100.0 |
| 354 | L | 98.4 |
| 355 | M | 96.7 |
| 356 | A | 77.0 |
| 357 | F | 9.8 |
| 358 | W | 100.0 |
| 359 | W | 100.0 |
| 360 | L | 88.5 |
| 361 | L | 78.7 |
| 362 | A | 96.7 |
| 363 | S | 95.1 |
| 364 | L | 98.4 |
| 365 | A | 31.1 |
| 366 | N | 100.0 |
| 367 | L | 98.4 |
| 368 | A | 93.4 |
| 369 | L | 98.4 |
| 370 | P | 100.0 |
| 371 | P | 100.0 |
| 372 | T | 78.7 |
| 373 | I | 100.0 |
| 374 | N | 100.0 |
| 375 | L | 100.0 |
| 376 | L | 27.9 |
| 377 | G | 98.4 |
| 378 | E | 100.0 |
| 379 | L | 96.7 |
| 380 | S | 11.5 |
| 381 | V | 63.9 |
| 382 | L | 13.1 |
| 383 | V | 19.7 |
| 384 | T | 14.8 |
| 385 | T | 26.2 |
| 386 | F | 98.4 |
| 387 | S | 95.1 |
| 388 | W | 100.0 |
| 389 | S | 98.4 |
| 390 | N | 86.9 |
| 391 | I | 36.1 |
| 392 | T | 82.0 |
| 393 | L | 9.8 |
| 394 | L | 23.0 |
| 395 | L | 91.8 |
| 396 | T | 24.6 |
| 397 | G | 96.7 |
| 398 | L | 44.3 |
| 399 | N | 100.0 |
| 400 | M | 42.6 |
| 401 | L | 42.6 |
| 402 | V | 3.3 |
| 403 | T | 100.0 |
| 404 | A | 85.2 |
| 405 | L | 82.0 |
| 406 | Y | 100.0 |
| 407 | S | 85.2 |
| 408 | L | 91.8 |
| 409 | Y | 86.9 |
| 410 | M | 100.0 |
| 411 | F | 19.7 |
| 412 | T | 27.9 |
| 413 | T | 63.9 |
| 414 | T | 86.9 |
| 415 | Q | 100.0 |
| 416 | W | 6.6 |
| 417 | G | 100.0 |
| 418 | S | 4.9 |
| 419 | L | 24.6 |
| 420 | T | 82.0 |
| 421 | H | 49.2 |
| 422 | H | 98.4 |
| 423 | I | 59.0 |
| 424 | N | 45.9 |
| 425 | N | 63.9 |
| 426 | M | 21.3 |
| 427 | K | 54.1 |
| 428 | P | 98.4 |
| 429 | S | 83.6 |
| 430 | F | 78.7 |
| 431 | T | 100.0 |
| 432 | R | 100.0 |
| 433 | E | 100.0 |
| 434 | N | 72.1 |
| 435 | T | 42.6 |
| 436 | L | 95.1 |
| 437 | M | 98.4 |
| 438 | F | 8.2 |
| 439 | M | 24.6 |
| 440 | H | 100.0 |
| 441 | L | 52.5 |
| 442 | S | 8.2 |
| 443 | P | 100.0 |
| 444 | I | 16.4 |
| 445 | L | 70.5 |
| 446 | L | 90.2 |
| 447 | L | 91.8 |
| 448 | S | 72.1 |
| 449 | L | 70.5 |
| 450 | N | 90.2 |
| 451 | P | 100.0 |
| 452 | D | 6.6 |
| 453 | I | 65.6 |
| 454 | I | 96.7 |
| 455 | T | 11.5 |
| 456 | G | 100.0 |
| 457 | F | 18.0 |
| 458 | S | 3.3 |
| 459 | S | 8.2 |

TABLE 11
ND4L gene

| AA# | AA | % cons. |
|---|---|---|
| 1 | M | 100.0 |
| 2 | P | 39.3 |
| 3 | L | 42.6 |
| 4 | I | 49.2 |
| 5 | Y | 55.7 |
| 6 | M | 42.6 |
| 7 | N | 100.0 |
| 8 | I | 62.3 |
| 9 | M | 23.0 |
| 10 | L | 62.3 |
| 11 | A | 100.0 |
| 12 | F | 93.4 |
| 13 | T | 42.6 |
| 14 | I | 26.2 |
| 15 | S | 73.8 |
| 16 | L | 88.5 |
| 17 | L | 47.5 |
| 18 | G | 100.0 |
| 19 | M | 26.2 |
| 20 | L | 88.5 |
| 21 | V | 18.0 |
| 22 | Y | 91.8 |
| 23 | R | 100.0 |
| 24 | S | 93.4 |
| 25 | H | 100.0 |
| 26 | L | 91.8 |
| 27 | M | 100.0 |
| 28 | S | 96.7 |
| 29 | S | 75.4 |
| 30 | L | 100.0 |
| 31 | L | 100.0 |
| 32 | C | 100.0 |
| 33 | L | 100.0 |
| 34 | E | 100.0 |
| 35 | G | 100.0 |
| 36 | M | 98.4 |
| 37 | M | 93.4 |
| 38 | L | 96.7 |
| 39 | S | 95.1 |
| 40 | L | 83.6 |
| 41 | F | 96.7 |
| 42 | I | 62.3 |
| 43 | M | 55.7 |
| 44 | A | 24.6 |
| 45 | T | 54.1 |
| 46 | L | 57.4 |
| 47 | M | 32.8 |
| 48 | T | 19.7 |
| 49 | L | 86.9 |
| 50 | N | 80.3 |
| 51 | T | 26.2 |
| 52 | H | 85.2 |
| 53 | S | 18.0 |
| 54 | L | 14.8 |
| 55 | L | 75.4 |
| 56 | A | 55.7 |
| 57 | N | 31.1 |
| 58 | I | 18.0 |
| 59 | V | 8.2 |
| 60 | P | 100.0 |
| 61 | I | 75.4 |
| 62 | A | 3.3 |
| 63 | M | 13.3 |
| 64 | L | 98.4 |
| 65 | V | 100.0 |
| 66 | F | 100.0 |
| 67 | A | 86.9 |
| 68 | A | 100.0 |
| 69 | C | 98.4 |
| 70 | E | 98.4 |
| 71 | A | 96.7 |
| 72 | A | 88.5 |
| 73 | V | 54.1 |
| 74 | G | 100.0 |
| 75 | L | 100.0 |
| 76 | A | 60.7 |
| 77 | L | 98.4 |
| 78 | L | 100.0 |
| 79 | V | 93.4 |
| 80 | S | 16.4 |
| 81 | I | 36.1 |
| 82 | S | 98.4 |
| 83 | N | 93.4 |
| 84 | T | 85.2 |
| 85 | Y | 96.7 |
| 86 | G | 100.0 |
| 87 | L | 29.5 |
| 88 | D | 100.0 |
| 89 | Y | 83.6 |
| 90 | V | 88.5 |
| 91 | H | 9.8 |
| 92 | N | 95.1 |
| 93 | L | 100.0 |
| 94 | N | 98.4 |
| 95 | L | 96.7 |
| 96 | L | 100.0 |
| 97 | Q | 96.7 |
| 98 | C | 100.0 |

TABLE 12
ND5 gene

| AA# | AA | % cons. |
|---|---|---|
| 1 | M | 89.5 |
| 2 | T | 7.0 |
| 3 | M | 33.3 |
| 4 | H | 3.3 |
| 5 | T | 36.1 |
| 6 | T | 26.2 |
| 7 | M | 14.8 |
| 8 | T | 29.5 |
| 9 | T | 9.8 |
| 10 | L | 49.2 |
| 11 | T | 47.5 |
| 12 | L | 75.4 |
| 13 | T | 19.7 |
| 14 | S | 13.1 |
| 15 | L | 98.4 |
| 16 | I | 21.3 |
| 17 | P | 9.8 |
| 18 | P | 100.0 |
| 19 | I | 77.0 |
| 20 | L | 19.7 |
| 21 | T | 18.0 |
| 22 | T | 34.4 |
| 23 | L | 31.1 |
| 24 | V | 4.9 |
| 25 | N | 45.9 |
| 26 | P | 24.6 |
| 27 | N | 21.3 |
| 28 | K | 75.0 |
| 29 | K | 14.8 |
| 30 | N | 29.5 |
| 31 | S | 23.3 |
| 32 | Y | 63.9 |
| 33 | P | 90.2 |
| 34 | H | 32.8 |
| 35 | Y | 82.0 |
| 36 | V | 80.3 |
| 37 | K | 93.4 |
| 38 | S | 13.1 |
| 39 | I | 19.7 |
| 40 | V | 44.3 |
| 41 | A | 18.0 |
| 42 | S | 16.4 |
| 43 | T | 8.2 |
| 44 | F | 96.7 |
| 45 | I | 31.1 |
| 46 | I | 49.2 |
| 47 | S | 98.4 |
| 48 | L | 70.5 |
| 49 | F | 9.8 |
| 50 | P | 88.5 |
| 51 | T | 50.8 |
| 52 | T | 24.6 |
| 53 | M | 60.7 |
| 54 | F | 88.5 |
| 55 | M | 14.8 |
| 56 | C | 11.5 |
| 57 | L | 13.1 |
| 58 | D | 9.8 |
| 59 | Q | 72.1 |
| 60 | E | 91.8 |
| 61 | V | 9.8 |
| 62 | I | 47.5 |
| 63 | I | 80.3 |
| 64 | S | 70.5 |
| 65 | N | 88.5 |
| 66 | W | 98.4 |
| 67 | H | 86.9 |
| 68 | W | 98.4 |
| 69 | A | 8.2 |
| 70 | T | 77.0 |
| 71 | T | 18.0 |
| 72 | Q | 72.1 |
| 73 | T | 78.7 |
| 74 | T | 6.6 |
| 75 | Q | 9.8 |
| 76 | L | 88.5 |
| 77 | S | 57.4 |
| 78 | L | 63.9 |
| 79 | S | 90.2 |
| 80 | F | 91.8 |
| 81 | K | 100.0 |
| 82 | L | 72.1 |
| 83 | D | 100.0 |
| 84 | Y | 72.1 |
| 85 | F | 96.7 |
| 86 | S | 98.4 |
| 87 | M | 50.8 |
| 88 | M | 27.9 |
| 89 | F | 100.0 |
| 90 | I | 27.9 |
| 91 | P | 90.2 |
| 92 | V | 75.4 |
| 93 | A | 98.4 |
| 94 | L | 98.4 |
| 95 | F | 82.0 |
| 96 | V | 85.2 |
| 97 | T | 100.0 |
| 98 | W | 100.0 |
| 99 | S | 96.7 |
| 100 | I | 100.0 |
| 101 | M | 85.2 |
| 102 | E | 95.1 |
| 103 | F | 96.7 |
| 104 | S | 100.0 |
| 105 | L | 37.7 |
| 106 | W | 100.0 |
| 107 | Y | 100.0 |
| 108 | M | 100.0 |
| 109 | N | 8.2 |
| 110 | S | 93.4 |
| 111 | D | 100.0 |
| 112 | P | 95.1 |
| 113 | N | 52.5 |
| 114 | I | 95.1 |
| 115 | N | 78.7 |
| 116 | Q | 37.7 |
| 117 | F | 100.0 |
| 118 | F | 86.9 |
| 119 | K | 98.4 |
| 120 | Y | 96.7 |
| 121 | L | 100.0 |
| 122 | L | 85.2 |
| 123 | I | 21.3 |
| 124 | F | 100.0 |
| 125 | L | 100.0 |
| 126 | I | 86.9 |
| 127 | T | 96.7 |
| 128 | M | 100.0 |
| 129 | L | 41.0 |
| 130 | I | 93.4 |
| 131 | L | 100.0 |
| 132 | V | 85.2 |
| 133 | T | 82.0 |
| 134 | A | 96.7 |
| 135 | N | 98.4 |
| 136 | N | 100.0 |
| 137 | L | 83.6 |
| 138 | F | 88.5 |
| 139 | Q | 100.0 |
| 140 | L | 98.4 |
| 141 | F | 98.4 |
| 142 | I | 96.7 |
| 143 | G | 100.0 |
| 144 | W | 100.0 |
| 145 | E | 100.0 |
| 146 | G | 100.0 |
| 147 | V | 96.7 |
| 148 | G | 100.0 |
| 149 | I | 98.4 |
| 150 | M | 100.0 |
| 151 | S | 100.0 |
| 152 | F | 100.0 |
| 153 | L | 86.9 |
| 154 | L | 100.0 |
| 155 | I | 100.0 |
| 156 | S | 16.4 |
| 157 | W | 100.0 |
| 158 | W | 100.0 |
| 159 | Y | 60.7 |
| 160 | A | 18.0 |
| 161 | R | 98.4 |
| 162 | A | 16.4 |
| 163 | D | 91.8 |
| 164 | A | 100.0 |
| 165 | N | 100.0 |
| 166 | T | 100.0 |
| 167 | A | 100.0 |
| 168 | A | 100.0 |
| 169 | I | 14.8 |
| 170 | Q | 100.0 |
| 171 | A | 100.0 |
| 172 | V | 24.6 |
| 173 | L | 96.7 |
| 174 | Y | 96.7 |
| 175 | N | 100.0 |
| 176 | R | 100.0 |
| 177 | I | 98.4 |
| 178 | G | 100.0 |
| 179 | D | 100.0 |
| 180 | I | 82.0 |
| 181 | G | 100.0 |
| 182 | F | 88.5 |
| 183 | I | 65.6 |
| 184 | L | 65.6 |
| 185 | A | 54.1 |
| 186 | L | 13.1 |
| 187 | A | 85.2 |
| 188 | W | 100.0 |
| 189 | F | 86.9 |
| 190 | I | 3.3 |
| 191 | L | 39.3 |
| 192 | H | 31.1 |
| 193 | S | 34.4 |
| 194 | N | 95.1 |
| 195 | S | 63.9 |
| 196 | W | 96.7 |
| 197 | D | 52.5 |
| 198 | P | 8.2 |
| 199 | Q | 96.7 |
| 200 | Q | 93.4 |
| 201 | M | 19.7 |
| 202 | A | 1.6 |
| 203 | L | 26.2 |
| 204 | L | 55.7 |
| 205 | N | 47.5 |
| 206 | A | 2.0 |
| 207 | N | 48.1 |
| 208 | P | 18.3 |
| 209 | S | 16.9 |
| 210 | L | 24.6 |
| 211 | T | 6.6 |
| 212 | P | 91.8 |
| 213 | L | 98.4 |
| 214 | L | 44.3 |
| 215 | G | 98.4 |
| 216 | L | 95.1 |
| 217 | L | 68.9 |
| 218 | L | 85.2 |
| 219 | A | 100.0 |
| 220 | A | 98.4 |
| 221 | A | 27.9 |
| 222 | G | 100.0 |
| 223 | K | 100.0 |
| 224 | S | 100.0 |
| 225 | A | 100.0 |
| 226 | Q | 100.0 |
| 227 | L | 11.5 |
| 228 | G | 98.4 |
| 229 | L | 100.0 |
| 230 | H | 100.0 |
| 231 | P | 100.0 |
| 232 | W | 100.0 |
| 233 | L | 100.0 |
| 234 | P | 100.0 |
| 235 | S | 91.8 |
| 236 | A | 100.0 |
| 237 | M | 100.0 |
| 238 | E | 100.0 |
| 239 | G | 100.0 |
| 240 | P | 100.0 |
| 241 | T | 100.0 |
| 242 | P | 100.0 |
| 243 | V | 100.0 |
| 244 | S | 100.0 |
| 245 | A | 100.0 |
| 246 | L | 100.0 |
| 247 | L | 100.0 |
| 248 | H | 100.0 |
| 249 | S | 100.0 |
| 250 | S | 100.0 |
| 251 | T | 100.0 |
| 252 | M | 100.0 |
| 253 | V | 100.0 |
| 254 | V | 98.4 |
| 255 | A | 100.0 |
| 256 | G | 100.0 |
| 257 | I | 45.9 |
| 258 | F | 100.0 |
| 259 | L | 98.4 |
| 260 | L | 95.1 |
| 261 | I | 88.5 |
| 262 | R | 100.0 |
| 263 | F | 100.0 |
| 264 | H | 45.9 |
| 265 | P | 96.7 |
| 266 | L | 80.3 |
| 267 | A | 6.6 |
| 268 | E | 78.7 |
| 269 | N | 65.6 |
| 270 | S | 4.9 |
| 271 | P | 16.4 |
| 272 | L | 24.6 |
| 273 | I | 57.4 |
| 274 | Q | 72.1 |
| 275 | T | 88.5 |
| 276 | L | 41.0 |
| 277 | T | 55.7 |
| 278 | L | 95.1 |
| 279 | C | 96.7 |
| 280 | L | 91.8 |
| 281 | G | 100.0 |
| 282 | A | 100.0 |
| 283 | I | 75.4 |
| 284 | T | 100.0 |
| 285 | T | 100.0 |
| 286 | L | 100.0 |
| 287 | F | 100.0 |
| 288 | A | 13.1 |
| 289 | A | 98.4 |
| 290 | V | 8.2 |
| 291 | C | 100.0 |
| 292 | A | 100.0 |
| 293 | L | 85.2 |
| 294 | T | 98.4 |
| 295 | Q | 100.0 |
| 296 | N | 100.0 |
| 297 | D | 100.0 |
| 298 | I | 96.7 |
| 299 | K | 98.4 |
| 300 | K | 100.0 |
| 301 | I | 96.7 |
| 302 | V | 57.4 |
| 303 | A | 98.4 |
| 304 | F | 98.4 |
| 305 | S | 100.0 |
| 306 | T | 100.0 |
| 307 | S | 100.0 |
| 308 | S | 100.0 |
| 309 | Q | 100.0 |
| 310 | L | 100.0 |
| 311 | G | 100.0 |
| 312 | L | 100.0 |
| 313 | M | 100.0 |
| 314 | M | 78.7 |
| 315 | V | 96.7 |
| 316 | T | 98.4 |
| 317 | I | 85.2 |
| 318 | G | 100.0 |
| 319 | I | 78.7 |
| 320 | N | 98.4 |
| 321 | Q | 100.0 |
| 322 | P | 100.0 |
| 323 | H | 62.3 |
| 324 | L | 96.7 |
| 325 | A | 100.0 |
| 326 | F | 98.4 |
| 327 | L | 91.8 |
| 328 | H | 100.0 |
| 329 | I | 96.7 |
| 330 | C | 100.0 |
| 331 | T | 95.1 |
| 332 | H | 100.0 |
| 333 | A | 100.0 |
| 334 | F | 98.4 |
| 335 | F | 100.0 |
| 336 | K | 100.0 |
| 337 | A | 100.0 |
| 338 | M | 95.1 |
| 339 | L | 100.0 |
| 340 | F | 100.0 |
| 341 | M | 62.3 |
| 342 | C | 96.7 |
| 343 | S | 96.7 |
| 344 | G | 100.0 |
| 345 | S | 100.0 |
| 346 | I | 98.4 |
| 347 | I | 100.0 |
| 348 | H | 100.0 |
| 349 | N | 54.1 |
| 350 | L | 98.4 |
| 351 | N | 93.4 |
| 352 | N | 34.4 |
| 353 | E | 100.0 |
| 354 | Q | 100.0 |
| 355 | D | 100.0 |
| 356 | I | 100.0 |
| 357 | R | 100.0 |
| 358 | K | 100.0 |
| 359 | M | 98.4 |
| 360 | G | 100.0 |
| 361 | G | 95.1 |
| 362 | L | 93.4 |
| 363 | L | 21.3 |
| 364 | K | 78.7 |
| 365 | T | 44.3 |
| 366 | M | 57.4 |
| 367 | P | 100.0 |
| 368 | L | 21.3 |
| 369 | T | 100.0 |
| 370 | S | 54.1 |
| 371 | T | 60.7 |
| 372 | S | 41.0 |
| 373 | L | 95.1 |
| 374 | T | 19.7 |
| 375 | I | 70.5 |
| 376 | G | 96.7 |
| 377 | S | 83.6 |
| 378 | L | 96.7 |
| 379 | A | 98.4 |
| 380 | L | 100.0 |
| 381 | A | 8.2 |
| 382 | G | 100.0 |
| 383 | M | 70.5 |
| 384 | P | 100.0 |
| 385 | F | 95.1 |
| 386 | L | 100.0 |
| 387 | T | 83.6 |
| 388 | G | 100.0 |
| 389 | F | 100.0 |
| 390 | Y | 100.0 |
| 391 | S | 100.0 |
| 392 | K | 100.0 |
| 393 | D | 100.0 |
| 394 | H | 1.6 |
| 395 | I | 100.0 |
| 396 | I | 100.0 |
| 397 | E | 100.0 |
| 398 | T | 36.1 |
| 399 | A | 68.9 |
| 400 | N | 85.2 |
| 401 | M | 16.4 |
| 402 | S | 95.1 |
| 403 | Y | 80.3 |
| 404 | T | 83.6 |
| 405 | N | 100.0 |
| 406 | A | 88.5 |
| 407 | W | 100.0 |
| 408 | A | 100.0 |
| 409 | L | 98.4 |
| 410 | S | 18.0 |
| 411 | I | 42.6 |
| 412 | T | 98.4 |
| 413 | L | 93.4 |
| 414 | I | 65.6 |
| 415 | A | 100.0 |
| 416 | T | 98.4 |
| 417 | S | 90.2 |
| 418 | L | 67.2 |
| 419 | T | 100.0 |
| 420 | S | 13.1 |
| 421 | A | 34.4 |
| 422 | Y | 100.0 |
| 423 | S | 100.0 |
| 424 | T | 80.3 |
| 425 | R | 100.0 |
| 426 | M | 16.4 |
| 427 | I | 80.3 |
| 428 | L | 14.8 |
| 429 | L | 16.4 |
| 430 | T | 31.1 |
| 431 | L | 88.5 |
| 432 | T | 14.8 |
| 433 | G | 67.2 |
| 434 | Q | 73.8 |
| 435 | P | 100.0 |
| 436 | R | 95.1 |
| 437 | F | 78.7 |
| 438 | P | 50.8 |
| 439 | T | 39.3 |
| 440 | L | 47.5 |
| 441 | T | 23.0 |
| 442 | N | 18.0 |
| 443 | I | 95.1 |
| 444 | N | 98.4 |
| 445 | E | 100.0 |
| 446 | N | 100.0 |
| 447 | N | 85.2 |
| 448 | P | 90.2 |
| 449 | T | 11.5 |
| 450 | L | 95.1 |
| 451 | L | 19.7 |
| 452 | N | 95.1 |
| 453 | P | 55.7 |
| 454 | I | 98.4 |
| 455 | K | 85.2 |
| 456 | R | 100.0 |
| 457 | L | 100.0 |
| 458 | A | 32.8 |
| 459 | A | 4.9 |
| 460 | G | 98.4 |
| 461 | S | 98.4 |
| 462 | L | 14.8 |
| 463 | F | 83.6 |
| 464 | A | 91.8 |
| 465 | G | 98.4 |
| 466 | F | 82.0 |
| 467 | L | 47.5 |
| 468 | I | 77.0 |
| 469 | T | 27.9 |
| 470 | N | 50.8 |
| 471 | N | 75.4 |
| 472 | I | 85.2 |
| 473 | S | 1.6 |
| 474 | P | 100.0 |
| 475 | A | 1.6 |
| 476 | S | 27.9 |
| 477 | P | 3.3 |
| 478 | F | 1.6 |
| 479 | Q | 57.4 |
| 480 | T | 18.0 |
| 481 | T | 98.4 |
| 482 | I | 9.8 |
| 483 | P | 100.0 |
| 484 | L | 36.1 |
| 485 | Y | 73.8 |
| 486 | L | 73.8 |
| 487 | K | 100.0 |
| 488 | L | 55.7 |
| 489 | T | 62.3 |
| 490 | A | 98.4 |
| 491 | L | 93.4 |
| 492 | A | 23.0 |
| 493 | V | 70.5 |
| 494 | T | 98.4 |
| 495 | F | 4.9 |
| 496 | L | 73.8 |
| 497 | G | 100.0 |
| 498 | L | 24.6 |
| 499 | L | 26.2 |
| 500 | T | 9.8 |
| 501 | A | 95.1 |
| 502 | L | 54.1 |
| 503 | D | 18.0 |
| 504 | L | 85.2 |
| 505 | N | 73.8 |
| 506 | Y | 9.8 |
| 507 | L | 36.1 |
| 508 | T | 77.0 |
| 509 | N | 29.5 |
| 510 | K | 24.6 |
| 511 | L | 95.1 |
| 512 | K | 83.6 |
| 513 | M | 18.0 |
| 514 | K | 42.6 |
| 515 | S | 8.3 |
| 516 | P | 63.9 |
| 517 | L | 3.3 |
| 518 | C | 1.6 |
| 519 | T | 27.9 |
| 520 | F | 52.5 |
| 521 | Y | 11.5 |
| 522 | F | 100.0 |
| 523 | S | 98.4 |
| 524 | N | 63.9 |
| 525 | M | 41.0 |
| 526 | L | 100.0 |
| 527 | G | 100.0 |
| 528 | F | 27.9 |
| 529 | Y | 37.7 |
| 530 | P | 86.9 |
| 531 | S | 11.5 |
| 532 | I | 75.4 |
| 533 | T | 9.8 |
| 534 | H | 98.4 |
| 535 | R | 100.0 |
| 536 | T | 9.8 |
| 537 | I | 21.3 |
| 538 | P | 91.8 |
| 539 | Y | 24.6 |
| 540 | L | 23.0 |
| 541 | G | 9.8 |
| 542 | L | 96.7 |
| 543 | L | 29.5 |
| 544 | T | 13.1 |
| 545 | S | 85.2 |
| 546 | Q | 90.2 |
| 547 | N | 36.1 |
| 548 | L | 41.0 |
| 549 | P | 6.6 |
| 550 | L | 18.0 |
| 551 | L | 25.0 |
| 552 | L | 85.2 |
| 553 | L | 77.0 |
| 554 | D | 100.0 |
| 555 | L | 59.0 |
| 556 | T | 32.8 |
| 557 | W | 98.4 |
| 558 | L | 77.0 |
| 559 | E | 98.4 |
| 560 | K | 55.7 |
| 561 | L | 14.8 |
| 562 | L | 50.8 |
| 563 | P | 100.0 |
| 564 | K | 96.7 |
| 565 | T | 27.9 |
| 566 | I | 59.0 |
| 567 | S | 63.9 |
| 568 | Q | 23.0 |
| 569 | H | 9.8 |
| 570 | Q | 75.4 |
| 571 | I | 14.8 |
| 572 | S | 24.6 |
| 573 | T | 18.0 |
| 574 | S | 90.2 |
| 575 | I | 23.0 |
| 576 | I | 4.9 |
| 577 | T | 21.3 |
| 578 | S | 82.0 |
| 579 | T | 19.7 |
| 580 | Q | 96.7 |
| 581 | K | 86.9 |
| 582 | G | 98.4 |
| 583 | M | 19.7 |
| 584 | I | 82.0 |
| 585 | K | 100.0 |
| 586 | L | 78.7 |
| 587 | Y | 100.0 |
| 588 | F | 95.1 |
| 589 | L | 85.2 |
| 590 | S | 91.8 |
| 591 | F | 95.1 |
| 592 | F | 11.5 |
| 593 | F | 9.8 |
| 594 | P | 16.4 |
| 595 | L | 56.7 |
| 596 | I | 25.0 |
| 597 | L | 68.3 |
| 598 | T | 15.0 |
| 599 | L | 45.0 |
| 600 | L | 46.7 |
| 601 | L | 56.7 |
| 602 | I | 31.7 |
| 603 | T | 17.9 |

TABLE 13
ND6 gene

| AA# | AA | % cons. |
|---|---|---|
| 1 | M | 70.5 |
| 2 | M | 29.4 |
| 3 | Y | 82.0 |
| 4 | A | 8.2 |
| 5 | L | 19.7 |
| 6 | F | 77.0 |
| 7 | L | 41.0 |
| 8 | L | 85.2 |
| 9 | S | 100.0 |
| 10 | V | 44.3 |
| 11 | G | 3.3 |
| 12 | L | 29.5 |
| 13 | V | 85.2 |
| 14 | M | 29.5 |
| 15 | G | 72.1 |
| 16 | F | 91.8 |
| 17 | V | 75.4 |
| 18 | G | 78.7 |
| 19 | F | 83.6 |
| 20 | S | 80.3 |
| 21 | S | 86.9 |
| 22 | K | 98.4 |
| 23 | P | 100.0 |
| 24 | S | 100.0 |
| 25 | P | 100.0 |
| 26 | I | 85.2 |
| 27 | Y | 98.4 |
| 28 | G | 100.0 |
| 29 | G | 100.0 |
| 30 | L | 83.6 |
| 31 | V | 29.5 |
| 32 | L | 100.0 |
| 33 | I | 77.0 |
| 34 | V | 62.3 |
| 35 | S | 90.2 |
| 36 | G | 100.0 |
| 37 | V | 11.5 |
| 38 | V | 72.1 |
| 39 | G | 100.0 |
| 40 | C | 98.4 |
| 41 | V | 4.9 |
| 42 | I | 73.8 |
| 43 | I | 27.9 |
| 44 | L | 63.9 |
| 45 | N | 37.7 |
| 46 | F | 52.5 |
| 47 | G | 86.9 |
| 48 | G | 90.2 |
| 49 | G | 11.5 |
| 50 | Y | 16.4 |
| 51 | M | 13.1 |
| 52 | G | 100.0 |
| 53 | L | 98.4 |
| 54 | M | 80.3 |
| 55 | V | 93.4 |
| 56 | F | 100.0 |
| 57 | L | 100.0 |
| 58 | I | 72.1 |
| 59 | Y | 100.0 |
| 60 | L | 100.0 |
| 61 | G | 100.0 |
| 62 | G | 100.0 |
| 63 | M | 100.0 |
| 64 | M | 31.1 |
| 65 | V | 100.0 |
| 66 | V | 100.0 |
| 67 | F | 100.0 |
| 68 | G | 100.0 |
| 69 | Y | 100.0 |
| 70 | T | 100.0 |
| 71 | T | 78.7 |
| 72 | A | 100.0 |
| 73 | M | 98.4 |
| 74 | A | 96.7 |
| 75 | I | 16.4 |
| 76 | E | 93.4 |
| 77 | E | 57.4 |
| 78 | Y | 98.4 |
| 79 | P | 100.0 |
| 80 | E | 96.7 |
| 81 | A | 24.6 |
| 82 | W | 98.4 |
| 83 | G | 34.4 |
| 84 | S | 86.9 |
| 85 | G | 14.8 |
| 86 | V | 29.5 |
| 87 | E | 14.8 |
| 88 | V | 70.5 |
| 89 | L | 67.2 |
| 90 | V | 11.5 |
| 91 | S | 19.7 |
| 92 | V | 11.5 |
| 93 | L | 39.3 |
| 94 | V | 29.5 |
| 95 | G | 86.9 |
| 96 | L | 70.5 |
| 97 | A | 9.8 |
| 98 | M | 54.1 |
| 99 | E | 95.1 |
| 100 | V | 40.7 |
| 101 | G | 16.7 |
| 102 | L | 52.9 |
| 103 | V | 62.3 |
| 104 | L | 36.1 |
| 105 | W | 36.1 |
| 106 | V | 41.0 |
| 107 | K | 11.5 |
| 108 | E | 27.6 |
| 109 | Y | 40.0 |
| 110 | D | 60.0 |
| 111 | G | 34.5 |
| 112 | V | 32.1 |
| 113 | V | 52.5 |
| 114 | V | 23.3 |
| 115 | V | 38.3 |
| 116 | V | 63.8 |
| 117 | N | 15.1 |
| 118 | F | 79.2 |
| 119 | N | 48.1 |
| 120 | S | 25.9 |
| 121 | V | 19.7 |
| 122 | G | 72.1 |
| 123 | S | 16.7 |
| 124 | W | 83.6 |
| 125 | M | 16.4 |
| 126 | I | 50.8 |
| 127 | Y | 59.0 |
| 128 | E | 24.6 |
| 129 | G | 29.5 |
| 130 | E | 26.2 |
| 131 | G | 39.3 |
| 132 | S | 49.2 |
| 133 | G | 75.4 |
| 134 | L | 27.9 |
| 135 | I | 19.7 |
| 136 | R | 32.8 |
| 137 | E | 68.9 |
| 138 | D | 47.5 |
| 139 | P | 13.1 |
| 140 | I | 18.0 |
| 141 | G | 98.4 |
| 142 | A | 21.3 |
| 143 | G | 18.0 |
| 144 | A | 72.1 |
| 145 | L | 90.2 |
| 146 | Y | 96.7 |
| 147 | D | 18.0 |
| 148 | Y | 73.8 |
| 149 | G | 91.8 |
| 150 | R | 14.8 |
| 151 | W | 98.4 |
| 152 | L | 67.2 |
| 153 | V | 55.7 |
| 154 | V | 54.1 |
| 155 | V | 72.1 |
| 156 | T | 50.8 |
| 157 | G | 98.4 |
| 158 | W | 98.4 |
| 159 | T | 18.0 |
| 160 | L | 96.7 |
| 161 | F | 67.2 |
| 162 | V | 42.6 |
| 163 | G | 55.0 |
| 164 | V | 65.6 |
| 165 | Y | 23.0 |
| 166 | I | 45.9 |
| 167 | V | 50.8 |
| 168 | I | 41.0 |
| 169 | E | 98.4 |
| 170 | I | 61.7 |
| 171 | A | 11.7 |
| 172 | R | 100.0 |
| 173 | G | 91.5 |
| 174 | N | 50.9 |

TABLE 14
Determination of amino acid positions in a mitochondrial gene.

| | First Coding | Last Coding | | |
|---|---|---|---|---|
| | Nucleotide | Nucleotide | Amino | SEQ |
| Gene Name | Position (FCNP) | Position | Acids | ID NO: |
| ND1 | 3307 | 4260 | 1-318 | 2 |
| ND2 | 4470 | 5510 | 1-347 | 3 |
| CO1 | 5904 | 7442 | 1-513 | 4 |
| CO2 | 7586 | 8266 | 1-227 | 5 |
| ATP8 | 8366 | 8569 | 1-68 | 6 |
| ATP6 | 8527 | 9204 | 1-226 | 7 |
| CO3 | 9207 | 9989 | 1-261 | 8 |
| ND3 | 10059 | 10403 | 1-115 | 9 |
| ND4L | 10470 | 10763 | 1-98 | 10 |
| ND4 | 10760 | 12136 | 1-459 | 11 |
| ND5 | 12337 | 14145 | 1-603 | 12 |
| ND6 | 14673 | 14152 | 1-174 | 13 |
| CytB | 14747 | 15886 | 1-380 | 14 |

TABLE 15
Nucleotide positions of loops of tRNAs

| tRNA | D-loop | Anti-CD loop | T-loop |
|---|---|---|---|
| Phe | 590-598 | 609-615 | 628-636 |
| Val | 1615-1620 | 1631-1637 | 1652-1657 |
| Ile | 4275-4280 | 4290-4296 | 4312-4318 |
| Met | 4415-4419 | 4430-4436 | 4451-4456 |
| Trp | 5525-5531 | 5542-5548 | 5561-5568 |
| Asp | 7531-7535 | 7546-7552 | 7567-7572 |
| Lys | 8307-8311 | 8321-8327 | 8343-8351 |
| Gly | 10004-10008 | 10019-10025 | 10039-10046 |
| Arg | 10418-10422 | 10433-10439 | 10453-10457 |
| His | 12151-12155 | 12166-12172 | 12187-12193 |
| Ser (AGY) | 12214-12217 | 12224-12230 | 12245-12252 |
| Thr | 15900-15907 | 15917-15932 | 15937-15941 |
| Pro | 16010-16005 | 15994-15998 | 15974-15968 |
| Glu | 14729-14725 | 14714-14708 | 14693-14687 |
| Tyr | 5879-5874 | 5864-5858 | 5843-5839 |
| Cys | 5814-5810 | 5800-5794 | 5780-5773 |
| Asn | 5717-5708 | 5698-5692 | 5676-5670 |
| Ala | 5642-5638 | 5627-5621 | 5606-5600 |
| Leu (UUR) | 3243-3252 | 32633269 | 3285-3291 |
| Leu (CUN) | 12279-12285 | 12296-12302 | 12317-12323 |
| Ser (UCN) | 7502-7498 | 7486-7480 | 7465-7459 |
| Gln | 43874380 | 4369-4363 | 4384-4342 |

TABLE 16
Raw data from 321 patients

| | | # non- | | | |
|---|---|---|---|---|---|
| | | synonymous | # | | |
| | # non- | variants in | variants | Baseline | |
| Patient | synonymous | genes <=90% | tRNA | dyspnea | |
| # | variants | conserved | genes | score | RILT |
| 1 | 3 | 3 | 0 | 2 | YES |
| 2 | 3 | 2 | 0 | 0 | NO |
| 3 | 2 | 2 | 0 | 0 | NO |
| 4 | 0 | 0 | 0 | 3 | YES |
| 5 | 3 | 3 | 0 | 1 | NO |
| 6 | 8 | 7 | 0 | 1 | YES |
| 7 | 6 | 5 | 0 | 0 | NO |
| 8 | 3 | 2 | 0 | 1 | NO |
| 9 | 9 | 8 | 0 | 1 | NO |
| 10 | 2 | 2 | 0 | 1 | YES |
| 11 | 4 | 4 | 0 | 0 | NO |
| 12 | 2 | 2 | 0 | 3 | YES |
| 13 | 4 | 3 | 1 | 0 | NO |
| 14 | 8 | 5 | 1 | 3 | YES |
| 15 | 4 | 4 | 0 | 1 | NO |
| 16 | 8 | 7 | 0 | 0 | NO |
| 17 | 6 | 5 | 1 | 0 | NO |
| 18 | 3 | 3 | 0 | 0 | NO |
| 19 | 6 | 6 | 1 | 2 | YES |
| 20 | 2 | 2 | 0 | 2 | NO |
| 21 | 2 | 2 | 0 | 1 | NO |
| 22 | 10 | 9 | 1 | 0 | NO |
| 23 | 7 | 6 | 1 | 0 | NO |
| 24 | 3 | 2 | 0 | 0 | NO |
| 25 | 4 | 4 | 1 | 1 | YES |
| 26 | 5 | 5 | 1 | 1 | NO |
| 27 | 10 | 10 | 0 | 0 | NO |
| 28 | 7 | 7 | 1 | 1 | YES |
| 29 | 2 | 2 | 0 | 1 | NO |
| 30 | 9 | 8 | 1 | 0 | NO |
| 31 | 6 | 5 | 0 | 1 | NO |
| 32 | 2 | 2 | 0 | 0 | NO |
| 33 | 2 | 2 | 0 | 0 | NO |
| 34 | 2 | 2 | 0 | 1 | NO |
| 35 | 6 | 5 | 1 | 1 | NO |
| 36 | 2 | 2 | 0 | 1 | NO |
| 37 | 2 | 2 | 0 | 1 | YES |
| 38 | 4 | 4 | 0 | 0 | NO |
| 39 | 4 | 3 | 0 | 1 | YES |
| 40 | 10 | 9 | 1 | 2 | YES |
| 41 | 8 | 8 | 0 | 1 | YES |
| 42 | 7 | 7 | 0 | 1 | NO |
| 43 | 3 | 3 | 0 | 1 | NO |
| 44 | 2 | 2 | 0 | 1 | NO |
| 45 | 2 | 2 | 0 | 1 | NO |
| 46 | 6 | 6 | 2 | 0 | NO |
| 47 | 8 | 7 | 0 | 0 | NO |
| 48 | 2 | 2 | 0 | 0 | NO |
| 49 | 7 | 7 | 0 | 0 | NO |
| 50 | 9 | 8 | 1 | 0 | NO |
| 51 | 2 | 2 | 0 | 0 | NO |
| 52 | 8 | 8 | 0 | 2 | YES |
| 53 | 6 | 6 | 0 | 1 | NO |
| 54 | 3 | 3 | 0 | 1 | NO |
| 55 | 4 | 4 | 1 | 1 | NO |
| 56 | 9 | 9 | 0 | 0 | NO |
| 57 | 2 | 2 | 0 | 1 | NO |
| 58 | 7 | 6 | 0 | 1 | NO |
| 59 | 3 | 2 | 0 | 1 | NO |
| 60 | 3 | 3 | 0 | 0 | NO |
| 61 | 6 | 5 | 1 | 1 | NO |
| 62 | 7 | 6 | 1 | 0 | NO |
| 63 | 11 | 11 | 0 | 1 | NO |
| 64 | 4 | 4 | 1 | 0 | NO |
| 65 | 10 | 10 | 1 | 1 | YES |
| 66 | 3 | 3 | 0 | 0 | NO |
| 67 | 5 | 5 | 1 | 2 | YES |
| 68 | 6 | 5 | 0 | 1 | YES |
| 69 | 3 | 2 | 0 | 1 | YES |
| 70 | 2 | 2 | 1 | 0 | NO |
| 71 | 3 | 3 | 0 | 1 | NO |
| 72 | 9 | 9 | 0 | 1 | NO |
| 73 | 2 | 2 | 0 | 0 | NO |
| 74 | 3 | 2 | 0 | 0 | YES |
| 75 | 6 | 5 | 1 | 0 | NO |
| 76 | 6 | 5 | 1 | 1 | YES |
| 77 | 2 | 2 | 0 | 0 | NO |
| 78 | 7 | 6 | 0 | 1 | NO |
| 79 | 3 | 3 | 0 | 0 | NO |
| 80 | 3 | 2 | 0 | 0 | NO |
| 81 | 11 | 10 | 0 | 0 | YES |
| 82 | 3 | 3 | 0 | 1 | NO |
| 83 | 6 | 5 | 0 | 0 | NO |
| 84 | 3 | 3 | 0 | 0 | NO |
| 85 | 6 | 6 | 0 | 1 | NO |
| 86 | 2 | 2 | 0 | 1 | NO |
| 87 | 10 | 10 | 0 | 1 | NO |
| 88 | 7 | 7 | 1 | 2 | YES |
| 89 | 10 | 11 | 0 | 3 | YES |
| 90 | 2 | 2 | 0 | 1 | NO |
| 91 | 6 | 6 | 0 | 3 | YES |
| 92 | 6 | 5 | 0 | 1 | YES |
| 93 | 3 | 4 | 1 | 0 | NO |
| 94 | 2 | 2 | 0 | 0 | NO |
| 95 | 2 | 2 | 0 | 1 | NO |
| 96 | 14 | 13 | 0 | 0 | YES |
| 97 | 3 | 3 | 0 | 1 | NO |
| 98 | 3 | 3 | 0 | 0 | NO |
| 99 | 4 | 3 | 0 | 0 | NO |
| 100 | 8 | 8 | 1 | 0 | NO |
| 101 | 9 | 9 | 1 | 1 | NO |
| 102 | 4 | 3 | 0 | 0 | NO |
| 103 | 2 | 2 | 0 | 0 | NO |
| 104 | 3 | 3 | 0 | 0 | NO |
| 105 | 7 | 6 | 2 | 0 | NO |
| 106 | 2 | 2 | 0 | 2 | NO |
| 107 | 2 | 2 | 0 | 0 | NO |
| 108 | 3 | 2 | 0 | 0 | NO |
| 109 | 2 | 2 | 1 | 1 | YES |
| 110 | 3 | 3 | 0 | 1 | NO |
| 111 | 10 | 9 | 1 | 0 | NO |
| 112 | 6 | 6 | 0 | 0 | NO |
| 113 | 3 | 4 | 0 | 0 | YES |
| 114 | 8 | 7 | 0 | 0 | NO |
| 115 | 2 | 2 | 0 | 2 | YES |
| 116 | 2 | 2 | 0 | 1 | NO |
| 117 | 6 | 5 | 0 | 0 | NO |
| 118 | 2 | 2 | 0 | 1 | YES |
| 119 | 2 | 2 | 1 | 1 | YES |
| 120 | 6 | 5 | 1 | 1 | NO |
| 121 | 6 | 5 | 1 | 1 | YES |
| 122 | 6 | 5 | 1 | 2 | NO |
| 123 | 2 | 2 | 0 | 1 | YES |
| 124 | 4 | 3 | 0 | 1 | NO |
| 125 | 4 | 4 | 0 | 1 | NO |
| 126 | 3 | 3 | 1 | 1 | YES |
| 127 | 7 | 6 | 1 | 0 | NO |
| 128 | 3 | 2 | 0 | 1 | NO |
| 129 | 9 | 6 | 1 | NA | NO |
| 130 | 2 | 2 | 0 | 0 | NO |
| 131 | 10 | 10 | 0 | 0 | NO |
| 132 | 3 | 3 | 0 | 0 | NO |
| 133 | 5 | 4 | 2 | 0 | NO |
| 134 | 2 | 2 | 0 | 0 | NO |
| 135 | 2 | 2 | 0 | 0 | NO |
| 136 | 3 | 3 | 0 | 0 | NO |
| 137 | 3 | 3 | 0 | 1 | NO |
| 138 | 7 | 6 | 1 | 0 | NO |
| 139 | 3 | 2 | 0 | 0 | NO |
| 140 | 8 | 7 | 0 | 1 | NO |
| 141 | 6 | 5 | 0 | 2 | YES |
| 142 | 2 | 2 | 1 | 0 | NO |
| 143 | 6 | 5 | 1 | 3 | NO |
| 144 | 5 | 3 | 2 | 2 | NO |
| 145 | 8 | 7 | 0 | 0 | NO |
| 146 | 3 | 3 | 0 | 1 | NO |
| 147 | 4 | 4 | 1 | 0 | NO |
| 148 | 2 | 2 | 0 | NA | NO |
| 149 | 6 | 5 | 0 | 0 | NO |
| 150 | 2 | 2 | 0 | 0 | NO |
| 151 | 8 | 8 | 0 | 0 | NO |
| 152 | 3 | 2 | 0 | 2 | NO |
| 153 | 2 | 2 | 0 | 1 | NO |
| 154 | 2 | 2 | 0 | 1 | NO |
| 155 | 5 | 5 | 1 | 1 | NO |
| 156 | 14 | 13 | 0 | 1 | NO |
| 157 | 7 | 6 | 1 | 0 | NO |
| 158 | 9 | 7 | 1 | 1 | NO |
| 159 | 3 | 3 | 0 | 0 | NO |
| 160 | 3 | 2 | 0 | 0 | NO |
| 161 | 2 | 2 | 0 | 0 | NO |
| 162 | 2 | 2 | 1 | 1 | NO |
| 163 | 3 | 2 | 0 | 0 | NO |
| 164 | 6 | 5 | 0 | 0 | YES |
| 165 | 6 | 5 | 1 | 1 | NO |
| 166 | 3 | 3 | 0 | 1 | NO |
| 167 | 6 | 5 | 0 | 1 | NO |
| 168 | 6 | 5 | 0 | 0 | NO |
| 169 | 6 | 5 | 1 | 1 | NO |
| 170 | 3 | 2 | 0 | 1 | NO |
| 171 | 7 | 6 | 0 | 0 | NO |
| 172 | 2 | 2 | 0 | 1 | NO |
| 173 | 7 | 7 | 0 | 2 | YES |
| 174 | 2 | 2 | 0 | 1 | NO |
| 175 | 8 | 7 | 1 | 0 | NO |
| 176 | 3 | 3 | 0 | 1 | NO |
| 177 | 9 | 8 | 1 | 2 | YES |
| 178 | 6 | 5 | 1 | 1 | YES |
| 179 | 3 | 2 | 0 | 1 | NO |
| 180 | 7 | 6 | 2 | 1 | YES |
| 181 | 2 | 2 | 0 | 2 | YES |
| 182 | 2 | 2 | 0 | 1 | NO |
| 183 | 3 | 3 | 0 | 0 | NO |
| 184 | 2 | 2 | 0 | 1 | NO |
| 185 | 2 | 2 | 1 | 1 | NO |
| 186 | 8 | 8 | 0 | 1 | NO |
| 187 | 2 | 2 | 0 | 0 | NO |
| 188 | 3 | 3 | 1 | 0 | YES |
| 189 | 6 | 5 | 0 | 2 | NO |
| 190 | 6 | 5 | 0 | 0 | NO |
| 191 | 2 | 2 | 0 | 1 | NO |
| 192 | 2 | 2 | 0 | 4 | YES |
| 193 | 2 | 2 | 0 | 1 | YES |
| 194 | 8 | 7 | 1 | 1 | YES |
| 195 | 2 | 2 | 0 | 0 | NO |
| 196 | 2 | 2 | 0 | 3 | NO |
| 197 | 3 | 2 | 0 | 1 | YES |
| 198 | 3 | 2 | 0 | 1 | NO |
| 199 | 4 | 4 | 1 | 3 | YES |
| 200 | 8 | 7 | 0 | 0 | YES |
| 201 | 11 | 8 | 0 | 0 | NO |
| 202 | 3 | 3 | 0 | 1 | NO |
| 203 | 7 | 6 | 1 | 0 | NO |
| 204 | 2 | 2 | 0 | NA | NO |
| 205 | 9 | 8 | 1 | 1 | YES |
| 206 | 4 | 4 | 1 | 0 | NO |
| 207 | 3 | 3 | 1 | 1 | YES |
| 208 | 9 | 8 | 1 | NA | NO |
| 209 | 6 | 5 | 1 | 1 | NO |
| 210 | 6 | 5 | 2 | 2 | YES |
| 211 | 10 | 10 | 0 | 1 | YES |
| 212 | 3 | 2 | 0 | 0 | NO |
| 213 | 2 | 2 | 0 | 1 | NO |
| 214 | 2 | 2 | 0 | 1 | NO |
| 215 | 2 | 2 | 0 | 0 | NO |
| 216 | 4 | 4 | 1 | 2 | YES |
| 217 | 4 | 4 | 1 | 0 | NO |
| 218 | 6 | 5 | 0 | 2 | NO |
| 219 | 3 | 2 | 0 | 1 | NO |
| 220 | 2 | 2 | 0 | 2 | NO |
| 221 | 5 | 4 | 1 | 2 | YES |
| 222 | 7 | 7 | 0 | 1 | YES |
| 223 | 8 | 5 | 1 | 1 | NO |
| 224 | 2 | 2 | 0 | 0 | NO |
| 225 | 3 | 3 | 0 | NA | NO |
| 226 | 9 | 9 | 0 | 1 | NO |
| 227 | 2 | 2 | 0 | 0 | NO |
| 228 | 2 | 2 | 1 | 0 | NO |
| 229 | 2 | 2 | 1 | 0 | NO |
| 230 | 7 | 7 | 1 | 3 | YES |
| 231 | 4 | 4 | 0 | 0 | NO |
| 232 | 6 | 5 | 1 | 1 | YES |
| 233 | 2 | 2 | 0 | 1 | NO |
| 234 | 2 | 2 | 0 | 1 | NO |
| 235 | 7 | 6 | 2 | 2 | YES |
| 236 | 2 | 2 | 0 | 0 | NO |
| 237 | 7 | 7 | 1 | 0 | YES |
| 238 | 7 | 6 | 0 | 1 | NO |
| 239 | 2 | 2 | 0 | 0 | NO |
| 240 | 3 | 3 | 0 | 0 | NO |
| 241 | 7 | 5 | 3 | 1 | NO |
| 242 | 2 | 2 | 0 | 0 | NO |
| 243 | 9 | 9 | 0 | 0 | NO |
| 244 | 3 | 2 | 0 | 1 | NO |
| 245 | 6 | 6 | 1 | 1 | YES |
| 246 | 2 | 2 | 0 | 1 | NO |
| 247 | 3 | 2 | 0 | 1 | NO |
| 248 | 3 | 3 | 0 | 0 | NO |
| 249 | 4 | 3 | 0 | 1 | NO |
| 250 | 6 | 4 | 1 | 1 | YES |
| 251 | 6 | 5 | 1 | 1 | YES |
| 252 | 6 | 5 | 0 | 2 | NO |
| 253 | 2 | 2 | 0 | 1 | NO |
| 254 | 9 | 9 | 0 | 3 | YES |
| 255 | 2 | 2 | 0 | 1 | NO |
| 256 | 0 | 0 | 0 | 1 | YES |
| 257 | 6 | 6 | 2 | 4 | YES |
| 258 | 11 | 8 | 0 | 1 | NO |
| 259 | 3 | 3 | 1 | 1 | NO |
| 260 | 4 | 3 | 1 | 1 | NO |
| 261 | 11 | 11 | 0 | 1 | NO |
| 262 | 6 | 5 | 0 | 0 | YES |
| 263 | 2 | 2 | 0 | 1 | YES |
| 264 | 5 | 4 | 2 | 1 | NO |
| 265 | 2 | 2 | 0 | 0 | NO |
| 266 | 7 | 7 | 1 | 0 | NO |
| 267 | 3 | 3 | 0 | 1 | NO |
| 268 | 7 | 6 | 2 | 2 | NO |
| 269 | 6 | 6 | 2 | 1 | NO |
| 270 | 6 | 6 | 2 | 1 | NO |
| 271 | 2 | 2 | 0 | 1 | YES |
| 272 | 2 | 2 | 0 | 1 | NO |
| 273 | 6 | 5 | 0 | 1 | YES |
| 274 | 2 | 2 | 0 | 0 | NO |
| 275 | 2 | 2 | 1 | 2 | NO |
| 276 | 2 | 2 | 0 | 1 | NO |
| 277 | 2 | 2 | 0 | 0 | NO |
| 278 | 5 | 4 | 1 | 1 | NO |
| 279 | 2 | 2 | 0 | 3 | YES |
| 280 | 2 | 2 | 1 | 1 | NO |
| 281 | 7 | 6 | 2 | 1 | NO |
| 282 | 3 | 3 | 0 | 1 | NO |
| 283 | 3 | 2 | 0 | 0 | NO |
| 284 | 2 | 2 | 0 | 0 | NO |
| 285 | 5 | 5 | 1 | 1 | NO |
| 286 | 5 | 5 | 1 | 1 | NO |
| 287 | 2 | 2 | 0 | 1 | NO |
| 288 | 7 | 6 | 1 | 1 | NO |
| 289 | 7 | 6 | 1 | 2 | YES |
| 290 | 8 | 7 | 1 | 0 | NO |
| 291 | 3 | 3 | 0 | 3 | YES |
| 292 | 2 | 2 | 0 | 1 | NO |
| 293 | 6 | 5 | 2 | 1 | YES |
| 294 | 6 | 5 | 1 | 2 | NO |
| 295 | 5 | 5 | 0 | 1 | NO |
| 296 | 3 | 3 | 0 | 0 | NO |
| 297 | 6 | 6 | 0 | 1 | NO |
| 298 | 3 | 3 | 0 | 0 | YES |
| 299 | 10 | 10 | 0 | 1 | NO |
| 300 | 6 | 6 | 0 | 1 | NO |
| 301 | 4 | 3 | 0 | 0 | NO |
| 302 | 3 | 3 | 0 | 0 | YES |
| 303 | 2 | 2 | 0 | 0 | NO |
| 304 | 8 | 8 | 0 | 2 | YES |
| 305 | 3 | 3 | 0 | 0 | NO |
| 306 | 2 | 2 | 0 | 0 | NO |
| 307 | 2 | 2 | 0 | 1 | NO |
| 308 | 10 | 10 | 0 | 0 | NO |
| 309 | 9 | 7 | 1 | 0 | NO |
| 310 | 7 | 5 | 1 | 0 | NO |
| 311 | 3 | 2 | 0 | 0 | NO |
| 312 | 8 | 6 | 1 | 0 | NO |
| 313 | 3 | 3 | 0 | 0 | NO |
| 314 | 2 | 2 | 0 | 1 | NO |
| 315 | 4 | 4 | 0 | 2 | YES |
| 316 | 5 | 5 | 1 | 0 | NO |
| 317 | 3 | 3 | 0 | 0 | NO |
| 318 | 6 | 5 | 1 | 1 | NO |
| 319 | 3 | 3 | 0 | 1 | NO |
| 320 | 4 | 3 | 1 | 0 | NO |
| 321 | 2 | 2 | 0 | 0 | NO |

### EXAMPLES

### Example 1

### Patient Population and DNA Isolation

Patient were recruited from MAASTRO Clinic (n=321) and Ghent University Hospital (n=66). Lung toxicity was scored using the Common Terminology Criteria for Adverse Events version 3.0 for dyspnea before (baseline) and up to six months after (maximum) radiotherapy (RT). The study was approved by the ethics committees of both centers, and all study participants provided written informed consent.

Total (nuclear and mitochondrial) cellular DNA was isolated from blood of patients collected before radiotherapy according to standard procedures.

Samples from patients with breast cancer were obtained from the University Medical Center of Mannheim. The skin biopsies were taken from patients treated in Mannheim as part of the TARGIT A phase 3 clinical trial [Vaidya et al., Lancet 382: 1-11 (2013)].

The TARGIT A trial was performed between 02/2002 and 12/2008, 305 patients were treated within TARGIT A (Arm A: n=34 IORT, n=20 IORT+WBRT for risk factors; Arm B WBRT: n=55) or received IORT as a planned boost (control group: n=196). Toxicity was assessed according to the LENT SOMA scales.

Patients were treated with interoperative radiation therapy (20 Gy/50 kV X-rays; INTRABEAM [Carl Zeiss Surgical, Oberkochen, Germany]) to the tumor bed during breast-conserving surgery as a boost followed by whole-breast radiotherapy (WBRT, 46 Gy). They underwent a prospective, predefined follow-up (median, 25 months; range 18-44 months), including clinical examination and breast ultrasound at 6-months and mammographies at 1-year intervals. Toxicities were documented using the common toxicity criteria (CTC)/European Organization for Research and Treatment of Cancer and the LENT-SOMA score. Cosmesis was evaluated with a score from 1 to 4.

### Example 2

### Sequence Determination

GeneChip® Mitochondria Resequencing 2.0 Arrays (Affymetrix, Santa Clara, Calif., USA) were used to determine the mtDNA sequence of the DNA samples according to the manufacturer's protocol.

After generation of the cell intensity (CEL) files by GeneChip® Operating Software 1.4 (GCOS 1.4), raw sequence data was analyzed by the Sequence Pilot—module SeqC (JSI medical systems) with the standard parameters. The eventual mtDNA sequences were compared with the revised Cambridge reference sequence (SEQ ID NO: 1) to list all homoplasmic variants. The haplogroups were determined as described previously (Voets, A. M., et al. Large scale mtDNA sequencing reveals sequence and functional conservation as major determinants of homoplasmic mtDNA variant distribution. Mitochondrion 11, 964-972 (2011)).

### Example 3

### Statistical Analysis

Multivariate logistic regression analyses models were built with α=0.05. The model performance for predicting outcome was evaluated by calculating the area under the curve of the receiver operating characteristic curve using a 10-fold cross-validation (out-of-sample) procedure. The maximum value of the AUC is 1.0, indicating a perfect discrimination, whereas 0.5 indicates a random chance to correctly discriminate outcome with the model. P values for AUCs being different from 0.5 were calculated using 500 bootstraps and a 1-sided Student's t-test. The final model output was represented by a nomogram (Iasonos, A., Schrag, D., Raj, G. V. & Panageas, K. S. How to build and interpret a nomogram for cancer prognosis. J Clin Oncol 26, 1364-1370 (2008)).

The endpoint for the model was the occurrence of dyspnea≧2 within six months after RT.

### Example 4

### Breast Cancer Patients

TABLE 21

| | Probability of radiation |
|---|---|
| Patient # | induced toxicity |
| 11 | 0.167 |
| 13 | 0.167 |
| 14 | 0.167 |
| 2 | 0.167 |
| 3 | 0.167 |
| 6 | 0.167 |
| 8 | 0.167 |
| 9 | 0.167 |
| 10 | 0.184 |
| 1 | 0.184 |
| 4 | 0.224 |
| 12 | 0.248 |
| 5 | 0.271 |
| 15 | 0.296 |
| 18 | 0.296 |
| 20 | 0.296 |
| 22 | 0.296 |
| 19 | 0.322 |
| 21 | 0.322 |
| 17 | 0.378 |
| 7 | 0.41 |

1. A method for treating a subject with radiation therapy, the method comprising:
treating the subject with radiation therapy;
wherein the risk of the subject developing radiation induced toxicity has been determined by a method comprising:
measuring the number of non-synonymous variations present in at least one gene encoding a mitochondrial protein in a sample of mitochondrial DNA from the subject, wherein said non-synonymous variations occur at positions that are less than 90% conserved, and
attributing a value to the number of non-synonymous variations, wherein a higher value corresponds to a higher risk for developing radiation induced toxicity.

2. The method according to claim 1 wherein the subject is diagnosed with lung cancer.

3. The method according to claim 1 wherein the subject is diagnosed with breast cancer.

4. The method according to claim 2, wherein the method of determining the risk of the subject developing radiation induced toxicity additionally comprises attributing a value to the baseline dyspnea score of the subject and adding that value to the value obtained from the number of non-synonymous variations to obtain an aggregated value, wherein a higher aggregated value corresponds to a higher risk of developing radiation induced toxicity, such as lung toxicity.

5. The method according to claim 1, wherein the method of determining the risk of the subject developing radiation induced toxicity additionally comprises determining the number of variations in tRNA loop positions, attributing a value to the number of variations in tRNA loop positions and adding that value to the value obtained from the number of non-synonymous variations or the aggregated value.

6. The method according to claim 1, wherein a value is attributed to a dose of chemotherapy that the subject is receiving wherein the value is proportional to the dose of chemotherapy, and adding that value to the value obtained from the number of non-synonymous variations or the aggregated value.

7. The method according to claim 1, wherein at least one gene encoding a mitochondrial protein is 2 3, 4, 5, 6, 7, 8, 9, 10, 11, 12 genes, or all genes encoding a mitochondrial protein.

8. The method according to claim 1, wherein the sample comprises nucleated cells.

9. The method according to claim 8, wherein the sample is a blood sample or a tissue sample.

10. The method according to claim 1, wherein the sample is taken from the subject before the start of the radiation therapy.

11. A method of radiation therapy, the method comprising:
measuring the number of non-synonymous variations present in at least one gene encoding a mitochondrial protein in a mitochondrial DNA sample from a subject, wherein said non-synonymous variations occur at positions that are less than 90% conserved, so as to attribute a value to the number of the non-synonymous variations, wherein a higher value of the non-synonymous variations corresponds to a greater risk for the subject to develop radiation induced toxicity, and
administering the highest possible dose of radiation to the subject to maximize radiation in view of the number of the subject's non-synonymous variations.
